SEQUENCE LISTING (93 sequences)

SEQ ID NO: 1 - DNA, 975 bp, Panicum virgatum
atgtgcggcggggccattctcagtgatctctactcaccagtgaggcggacggtcactgccggtgacctatggggagagagtggcagcagcaagaatgtgaagaactggaaaaggagttcttggaagtttgatgaaggcgatgaagactttgaagctgatttcaaggattttgaggattgcagtagcgaggaggaggtagattttggacatgaggaaaaagaattccaattgaacagttcgaatttcgtggaattcaatggccatactgccaaagtcaccagcaggaagcgaaagatccagtaccgagggatccggcggcggccttggggcaaatgggcagcagaaatcagagacccacagaagggcgtccgagtttggcttggcacgttcagcactgccgaggaagctgcaagggcatatgacgtggaagctctacgcatacgtggcaagaaagccaagatgaatttccctaccaccatcacagctgctgggaaacaccaccggcagcgtgtggctcgaccggcaaagaagacgtcacaagagagcctgaagtcaagcaatgcctctggtcatgtcatctcagcaggcagcagtactgatggcaccgttgtcaagatcgagttgtcacagtcaccagcttctccactaccagtgtccagcgcatggcttgatgcttttgagctgaagcagcttggtggagaaacccctgaagctgatgggagagaaacccctgaagaaactgatcatgaaacgggagtgacagcggatatgttttttggcaatggcgaagtgcggctttcagatgattttgcgtcttacgagccttacccaaattttatgcagttaccttatctagaaggtgactcgtatgaaaacattgacactcttttcaacggtgaagctgctcaggatggagtgaacatcggaggtctttggaatttcgatgatgtgccaatggaccgtggtgtttactga

SEQ ID NO: 2 - protein, 324 aa, Panicum virgatum
MCGGAILSDLYSPVRRTVTAGDLWGESGSSKNVKNWKRSSWKFDEGDEDFEADFKDFEDCSSEEEVDFGHEEKEFQLNSSNFVEFNGHTAKVTSRKRKIQYRGIRRRPWGKWAAEIRDPQKGVRVWLGTFSTAEEAARAYDVEALRIRGKKAKMNFPTTITAAGKHHRQRVARPAKKTSQESLKSSNASGHVISAGSSTDGTVVKIELSQSPASPLPVSSAWLDAFELKQLGGETPEADGRETPEETDHETGVTADMFFGNGEVRLSDDFASYEPYPNFMQLPYLEGDSYENIDTLFNGEAAQDGVNIGGLWNFDDVPMDRGVY

SEQ ID NO: 3 - DNA, 1086 bp, Zea mays
atgtgcggcggcgcgatccttgccgagctccgcgagccggcgccgcgccggctcacggagcgggacatctggcagcagaagaagaagcccaagaggggcggcgccggcgggaggcgctcgttcgcggcggaagacgatgaggacttcgaggccgacttcgaggactttgaagccgactccggtgattcggatttggagctcggggaaggggctgacgacgacgtcatcgagatcaagcccttcgccgccaagagtactttctccagagatggcttaagcaccatgactactgctggttatgatgcccctgcagcaaggttggccaaaaggaagaggaagaatcaatacaggggtatccgccagcgcccttggggtaagtgggctgctgagatcagagatccccagaagggcgttcgtgtttggcttggtactttcaatagtccggaggaagctgcaagagcttatgatgctgaagcgcgcaggattcgtggcaagaaggccaaggttaactttcctgatgcaccagcagttggtcagaagtgccgttctagttcagcttctgctaaagcactcaagtcatgtgttgaacagaagccaattgtcaaaacagatatgaacatccttgccaacacaaatgcacccttctaccaatctgttaactacgcatccaacaatccatttgttccagcaatgaactctactgtttcttttgaggatcctatcatgaatctgcactctgaccagggaagtaactcccttggctgctcagacttgggctgggagaatgatactaagacaccagacatcacatccattgctcccattcccactattgctgaaggcgatgagtctgtatttgtcaactccaattcaaacagctcgatggtgcctcctgtcctggagaacaatgctgttgatctcactgatgggctgacagatttggaatcctatatgaggtttcttatggatggcggtgcaagtgattcaattgatagccttctgaaccttgatggatcacaggatcttggtagcaatatggacctctggaccttcgatgacatgcccatcgctggcgatttcttctga

SEQ ID NO: 4 - protein, 361 aa, Zea mays
MCGGAILAELREPAPRRLTERDIWQQKKKPKRGGAGGRRSFAAEDDEDFEADFEDFEADSGDSDLELGEGADDDVIEIKPFAAKSTFSRDGLSTMTTAGYDAPAARLAKRKRKNQYRGIRQRPWGKWAAEIRDPQKGVRVWLGTFNSPEEAARAYDAEARRIRGKKAKVNFPDAPAVGQKCRSSSASAKALKSCVEQKPIVKTDMNILANTNAPFYQSVNYASNNPFVPAMNSTVSFEDPIMNLHSDQGSNSLGCSDLGWENDTKTPDITSIAPIPTIAEGDESVFVNSNSNSSMVPPVLENNAVDLTDGLTDLESYMRFLMDGGASDSIDSLLNLDGSQDLGSNMDLWTFDDMPIAGDFF

SEQ ID NO: 5 - DNA, 1092 bp, Zea mays
atgtgcggcggcgcgatccttgccaaccttcgcgagccggcgccgcgccggctcacagagcgggacatctggcagcagaaaaagaagctcaagaggggcggcggcggcgggaggcgctcgttcgcggcggaggacgatgaggacttcgaggccgactttgaggtcttcgaggccgactccagtgattcagatttggagctcagggaggggactgacgacgacgtcgtcgagatcaagcccttcactgccaagaggactttctccagcgatggcttaagcaccatgactagtgctgtagcaaggtcagccaagaggaagagaaagaatctatacaggggtatccgccagcggccttggggcaagtgggctgctgagatcagagatcctcagaagggtgttcgtgtttggcttggtactttcaatagtcctgaggaagctgcaagagcttatgatgccgaagcgcgcaggattcgtggcaagaaggccaaggttaactttcctgatgcaccagcagttggtcagaagcaccgttctggctcagcttctgctaaagcatccaagtcaagttttggacagaagcctatcgtcaaagtagctatgaacaaccttgccaacacaaatgcatccttcttccaatctgctagctacccctccaatttatttgttcagcatggcaatatgccatttgttccagcaatgaactctactgcttctgttgatgatcttatcatgaatctgcactctgaccagggaagtaactcctttggctgctcagacttgggctgggagaatgataccaagacaccagacatcacttccattgctcccatttccactattgcggaaggcgacgagtctgcatttgtcgacagcaattcaaacagctcatttgtgcttcctgccctggagaacagtgctgttgatctcactgatgggctgacagatttagaatcctatatgaggtttcttctggatggtggtccaagtgattcagttgatagccttctgaaccttgatggatcgcaggatgttggtagcgacatggacctctggagcttcgacgacatgcccatcgtcggcgatttcttttga

SEQ ID NO: 6 - protein, 363 aa, Zea mays
MCGGAILANLREPAPRRLTERDIWQQKKKLKRGGGGGRRSFAAEDDEDFEADFEVFEADSSDSDLELREGTDDDVVEIKPFTAKRTFSSDGLSTMTSAVARSAKRKRKNLYRGIRQRPWGKWAAEIRDPQKGVRVWLGTFNSPEEAARAYDAEARRIRGKKAKVNFPDAPAVGQKHRSGSASAKASKSSFGQKPIVKVAMNNLANTNASFFQSASYPSNLFVQHGNMPFVPAMNSTASVDDLIMNLHSDQGSNSFGCSDLGWENDTKTPDITSIAPISTIAEGDESAFVDSNSNSSFVLPALENSAVDLTDGLTDLESYMRFLLDGGPSDSVDSLLNLDGSQDVGSDMDLWSFDDMPIVGDFF

SEQ ID NO: 7 - DNA, 1092 bp, Zea mays
atgtgcggcggcgccatcctgtcggacatcatcccgccgccgccaccgcggcgggtcacggctggccacctctggcccgagagcaagaagccgaggagggctgcatccggcaggaggggagcccccgtggagcagcatgagcaggaggaggatttcgaggccgacttcgaggagttcgaggtggagtccggcgagtcggagctcgagtccgaggacgagcccaagcccttcgccgcccccaggagcgcgctcgccagaggtggactaaacactggtgcagctggtgtcgatggccctgctgcaaattcagttaaaaggaagaggaagaaccagttcaggggtatccgccggcgcccgtggggcaaatgggctgctgagatcagagatcctcgcaagggcgtgcgcgtctggctcggtactttcaactcccccgaagaagctgccagagcttacgacgccgaggcacgcaggatccgcggcaagaaggctaaagtcaacttcccggatgaggttcctacggcggtttctcagaagcgccgtgctgctgggcctgcctctctgaaagcgcctaagatggacgttgaggaggagaagccgatcatcaagctcgcagtgaacaatatgaccaactcaaacgcatatcactaccctgccgtcgtcggccacaacatcatacccgagccattcatgcagactcagaacatgccattcgctcctctggtgaattatgctgccctagtgaacctgtcttcagaccaaggcagcaactcgttcggttgctcggacttcagcctcgagaacgactccaggacccctgacataacttcggtgcctgcgcccgttgccaccttggccgccgttggcgagtctgtgttcgtccagaacaccgccggccatgctgtggcgtctcctgcgacggggaacactggtgttgatctcgccgagttggagccgtatatgaatttcctgatggacggtggttcagacgactcgatcagcactctcttgagctgtgatggatcccaggacgtggtcagcaacatggacctttggagcttcgaggacatgcccatgtctgctggtttctactga

SEQ ID NO: 8 - protein, 363 aa, Zea mays
MCGGAILSDIIPPPPPRRVTAGHLWPESKKPRRAASGRRGAPVEQHEQEEDFEADFEEFEVESGESELESEDEPKPFAAPRSALARGGLNTGAAGVDGPAANSVKRKRKNQFRGIRRRPWGKWAAEIRDPRKGVRVWLGTFNSPEEAARAYDAEARRIRGKKAKVNFPDEVPTAVSQKRRAAGPASLKAPKMDVEEEKPIIKLAVNNMTNSNAYHYPAVVGHNIIPEPFMQTQNMPFAPLVNYAALVNLSSDQGSNSFGCSDFSLENDSRTPDITSVPAPVATLAAVGESVFVQNTAGHAVASPATGNTGVDLAELEPYMNFLMDGGSDDSISTLLSCDGSQDVVSNMDLWSFEDMPMSAGFY

SEQ ID NO: 9 - DNA, 1023 bp, Zea mays
atgtgcggaggcgccatcctcgcggagctgatcccgccgacgcggcgcgtggcgtcgaagccggtgacagaaggccacctctggtcggcgagctccaagaaagccggcagcggcagggacaagaggcaccagcacgaatacgccgacgatgacttcgaggccgccttcgaggacttcgacgacgactttgacgtgcatgaagacgacgaggacggccacttcgtattctcgtccaaatccgccttgtccccagccctgcacgacgggcgcgcggcgagccagaagaagcagcgcgggcgccagttccgcggcatccggcagcggccctggggcaagtgggcggcggagatccgcgacccgcacaagggcacccgcgtctggctcggcaccttcagcaccgccgaggacgccgcccgggcctacgacgtggaggcgcgccgcctccgcggcagcaaggccaaggtcaacttccccgcagccagcggtcgcgctcgcggtcgcgcgcgcccacgccgcggcgacgacggcaacccacgaaccgcgccggaaacgcagcacccagcacagcccgctctgctgcctcgaggagagagagagacgcagaggaaggaagggatcgccgccgtgaagccagaagctacggagtcgttcgacgtgggcggcggtctcttcttcgacatggccttccccaccttcccagcctcgccgccgccgcaggccgtggatacgtccttcgccggcagcaccgccacgtcggagaccgggagccccgcgaagaggccgagatgcgacgaagactcgtccgagggcggcagcggctccgcgctggagctcgctgacgagctggcgttcgacccgtttgtgctgctgcagatgccctactcgggtgggtacgacgacgactcactggacggccttttcgccgcagatgaggccgtgcagcaggacgtgggcaacggcatggacggcgtccgcctgtggagcttcgacgagttccccgccgtcgacggttctgttttctga

SEQ ID NO: 10 - protein, 340 aa, Zea mays
MCGGAILAELIPPTRRVASKPVTEGHLWSASSKKAGSGRDKRHQHEYADDDFEAAFEDFDDDFDVHEDDEDGHFVFSSKSALSPALHDGRAASQKKQRGRQFRGIRQRPWGKWAAEIRDPHKGTRVWLGTFSTAEDAARAYDVEARRLRGSKAKVNFPAASGRARGRARPRRGDDGNPRTAPETQHPAQPALLPRGERETQRKEGIAAVKPEATESFDVGGGLFFDMAFPTFPASPPPQAVDTSFAGSTATSETGSPAKRPRCDEDSSEGGSGSALELADELAFDPFVLLQMPYSGGYDDDSLDGLFAADEAVQQDVGNGMDGVRLWSFDEFPAVDGSVF

SEQ ID NO: 11 - DNA, 744 bp, Panicum virgatum
atggcgtactacgacgtcggcgccggcgcggactcctccgccacctcgtcgcatcaacgaccgggccacctggccgccgcagccgtgccgccggcgcccgctggatctcatcggcaggcgactccggaagctggaactggaactggaactgctgctgctgctgccacggctggccagacagtggaggtgcaaggaggttacgggacgagaatgcactaccgtggcgtgcggcggcggccgtggggcaagtgggcggcggagatccgtgaccccgccaaggcggcgcgtgtgtggctcggcaccttcgacaccgcggaggccgccgccgcagcgtacgacgacgccgcgctccggttcaagggcgccaaggccaagctcaactttcccgagcgcgtccgcggccgtaccggccagggcgcgttcctcgtcagccctggcgtcccccagcagccgccgccgtcttccctgccaactgcagccgccgcgccgacgccgttccccggcttgatgcggtacgcgcaactccagggttggagcagcgggaacatcgcggccagcaacaccggtggtgatctcgcgccgccggcacaggcgtcgtcgtcggtgcagattctggacttctcgacgcagcaactactccggggctcaccgacaacgttcggcccaccgccgacgacgtcggcatcgatgtccaggactagcagagtagatgaggcgcacgagagttgcgatgctcctgactga

SEQ ID NO: 12 - protein, 247 aa, Panicum virgatum
MAYYDVGAGADSSATSSHQRPGHLAAAAVPPAPAGSHRQATPEAGTGTGTAAAAATAGQTVEVQGGYGTRMHYRGVRRRPWGKWAAEIRDPAKAARVWLGTFDTAEAAAAAYDDAALRFKGAKAKLNFPERVRGRTGQGAFLVSPGVPQQPPPSSLPTAAAAPTPFPGLMRYAQLQGWSSGNIAASNTGGDLAPPAQASSSVQILDFSTQQLLRGSPTTFGPPPTTSASMSRTSRVDEAHESCDAPD

SEQ ID NO: 13 - DNA, 1227 bp, Zea mays
atgctttttgcttcctccccaaagcaagcaaacctccataccgtaatcacagtgacaggcaccatcagtctctctccccgctcccgattgggtcagtcaaatacgcaaggaggtggtggtcaataccaagatcagattcagagcgagctgagggagaaaggcatcgtcatcgatcgatcgaaggtggatcatcggaggcatgacagtggcagcaagcgaaccctgcctgagcctgacggcgaggaggcggcggcggcggcggctcaatcgtcggcgtcgcggtcgctgggctcgagccaagagcgctacttcccgcgacggcggcagcagcagatagagctcggagggagtggtggtggtggtcacgatcacgatgaccgctactactcgcctgcggctgtgccggcaccgggacggtaccatgagcaggccgaacagcaaggtgctgtgtacagctccaccaccatggcgtactacgacgacgtgggcacgcactccgcctcctcatcgcttcagccgggcgacagcgccgccgccgccgtgccggtggccactgagtctcctcatcagcggcttccgctggaagccgggtctgctggttgttcctccgcggttgggcaagccgtggaggtgcaagaaggtactctgaccacgacgatgaggcactaccgtggcgtccgtcggcgaccatggggcaagtgggcggcggagatccgcgaccccgccaaagcggcgcgcgtctggctgggcaccttcgacacagcggaggccgccgcggctgcctacgaccgcgccgcgctccagttcaagggcgccaaggcaaaactcaacttccccgaacgcgtccggggacgcaccggccagggcgccttcctcgtatcccccggcatcccccagccgccgccagtatcagctccgttgttgccaccgtctccggtgccgttccccgacctgatgcggtacgcgcagttgcttcacagcggcaacgtagccgccgcctccgccagcaccgcccatgacctcgcgccgtcgtcgcagcaatcgccggtgcagattctggacttctctacgcgccgactcctcctgcgtggctcaccgcctgcaacgttcggccgaccgtcgatggcgtcctccaccgcggcttcttctaccagtatgccggtgcctcacgtcgaggacaaggatagcggagcaggcgaggagggtggcactgctccgcctgactga

SEQ ID NO: 14 - protein, 408 aa, Zea mays
MLFASSPKQANLHTVITVTGTISLSPRSRLGQSNTQGGGGQYQDQIQSELREKGIVIDRSKVDHRRHDSGSKRTLPEPDGEEAAAAAAQSSASRSLGSSQERYFPRRRQQQIELGGSGGGGHDHDDRYYSPAAVPAPGRYHEQAEQQGAVYSSTTMAYYDDVGTHSASSSLQPGDSAAAAVPVATESPHQRLPLEAGSAGCSSAVGQAVEVQEGTLTTTMRHYRGVRRRPWGKWAAEIRDPAKAARVWLGTFDTAEAAAAAYDRAALQFKGAKAKLNFPERVRGRTGQGAFLVSPGIPQPPPVSAPLLPPSPVPFPDLMRYAQLLHSGNVAAASASTAHDLAPSSQQSPVQILDFSTRRLLLRGSPPATFGRPSMASSTAASSTSMPVPHVEDKDSGAGEEGGTAPPD

SEQ ID NO: 15 - DNA, 1338 bp, Zea mays
atgcgcatctctctccgcgtacttatcagtagcgagctcggcacgtcattgtgcactgctgcaccgagcctagcgcgagcctccgtccgaaagtccaaatcttcagctctctccttgacgcatgcccggatcgatcggtccagctctacccaccggaggaggaggcagatcaacgggcagctgactaaggtggatccacggaggaggcatggcaagaggcccctacccgccgacgaggaggaggaagaggaggaggagctgcccccgccgccggcaaagtacgagcagctggatcaggaggagaagcatcacgttgtcgtctcgcagctgcaagcaggagctacctttagcggtggccgagggtcttcgtcgtcttccgtggccggcccttcgccggaggcgtacgcgcagtactactactcggcgcgcgccgaccacgacgcctccgccgtggcctccgcgctagcccatgtcatccgcgcctcgcccgaccaactcccgccgcagcaggccgcctgcttgtacggcgccgccggcgcgccggtcctgcggcagggagagggagaccatccgcaaccgcaagcggctgcgcaccaccatcccggtggccacgtcgccgccgaggaggagcaaggtgcaggtctgaggcggcactaccgaggggtgaggcagcggccgtggggcaagtgggcggcggagatccgggaccccaagaaggcggcgcgggtgtggctcggcaccttcgacacggcggaggacgccgccatcgcctacgacgaggcggcgctgcggttcaagggcaccaaggccaagctcaacttcccggagcgcgtgcagggccgaaccgacctcggcttcgtcgtcacccgcggcatcccggaccaccaccggcacccgcgggcggcggcggtgaacctggcagcaatgccgcaggcgcaggcgcagccgcacttgcagcacggccgcccgaccgtcatgccgtacccgtacccgtaccctgacctcatgcagtacgcgcagctgctgcagggcggccggggcggcggcgaccacgcggcggcggtccagcagcagctcatgatgatgggcgggcggggcggcaacctgcccttctcgttctcgccgccgtcgtcctggagtgcgccgccgcagatactggacttctcggcgcggcagctcatcacccagcccggcccgccgtcgtctccggccgcccccggcggcgcggcgccgtccacgccgtcgtccacgaccacggcgtcgtcgcccagcgccagcgccagcggcagtgcatggccgtacggtggggagcaccacaggaataaaaaggacgcgtga

SEQ ID NO: 16 - protein, 445 aa, Zea mays
MRISLRVLISSELGTSLCTAAPSLARASVRKSKSSALSLTHARIDRSSSTHRRRRQINGQLTKVDPRRRHGKRPLPADEEEEEEEELPPPPAKYEQLDQEEKHHVVVSQLQAGATFSGGRGSSSSSVAGPSPEAYAQYYYSARADHDASAVASALAHVIRASPDQLPPQQAACLYGAAGAPVLRQGEGDHPQPQAAAHHHPGGHVAAEEEQGAGLRRHYRGVRQRPWGKWAAEIRDPKKAARVWLGTFDTAEDAAIAYDEAALRFKGTKAKLNFPERVQGRTDLGFVVTRGIPDHHRHPRAAAVNLAAMPQAQAQPHLQHGRPTVMPYPYPYPDLMQYAQLLQGGRGGGDHAAAVQQQLMMMGGRGGNLPFSFSPPSSWSAPPQILDFSARQLITQPGPPSSPAAPGGAAPSTPSSTTTASSPSASASGSAWPYGGEHHRNKKDA

SEQ ID NO: 17 - DNA, 1269 bp, Zea mays
atggagcgcgtgaagtattgtgattgtactgtgtgcagtgtgcagcggtcattgtgttctacccgccggaggaggaggaggaggaggcagatcgaccggcagctgactaaggtggatccacggaggaggcatggcaagaggcccctacccgccgccgaggtggaagaggaggaggaggaggaggcgctgcccccgggcccgccgcccgcaaagcacgagcagctggaggagcctcaccacgccgccgtctcgcagctgcaaggagccacctttagcggcggcggagggtcgtcgtcgtcttccgtgatcggtggcccgtcgccgccgcaggcgtacgcgcagtactactactcggcgcgcgccgacaacgacgcctccgccgtggcctccgcgcttgcccacgtcatccgggcctcgcctgaccagcttccgccgcagcaggcgccggccttgtacggcgccggcgtcccgggcagccttcggctgggagaccacccgcaagcgtctgcgcaccactatcccggtcccggcggccacgtcgccgccgccgaggaggagcaaggtcggaggcggcactaccggggggtgaggcagcggccgtggggcaagtgggcggcagagatccgggaccccaagaaggcggcgcgggtgtggctcggcaccttcgacacggcggaggacgccgccatcgcctacgacgaggcggcgctgcggttcaagggcaccaaggccaagctcaacttcccggagcgcgtgcagggccgaaccgacctcggcttcctcgtcacccgcggcatcccggaccaccggcacccgtcggcggcggtgaccctggcagcaatgccgccgccgcaccaccagcacggccaccagaccgtcgtgccgtaccccgacctcatgcagtacgcgcagctgctgcagggcggccggggcggcggcggccacgccgaggcggcggtccagcaggcgcaccgtcagcagcagcagcagcagctcatgacgatgatgggcggtcggccgggagtcaacctgccctccacgttctcgccgtcgtcgtccgcatcggcgccgcagatactggacttctccacgcagcagctcatccggcccggcccgccgtcgccgtcgccgccgcgggccgcggcaatgccgtcctcgtcggccgctgcggcgccgtccacgccgtcgtccacgaccacagcgtcgtcgcccagcggcggtgcatggccgtacggcggggagcgccacaggaataaaaaagacgcgtga

SEQ ID NO: 18 - protein, 422 aa, Zea mays
MERVKYCDCTVCSVQRSLCSTRRRRRRRRQIDRQLTKVDPRRRHGKRPLPAAEVEEEEEEEALPPGPPPAKHEQLEEPHHAAVSQLQGATFSGGGGSSSSSVIGGPSPPQAYAQYYYSARADNDASAVASALAHVIRASPDQLPPQQAPALYGAGVPGSLRLGDHPQASAHHYPGPGGHVAAAEEEQGRRRHYRGVRQRPWGKWAAEIRDPKKAARVWLGTFDTAEDAAIAYDEAALRFKGTKAKLNFPERVQGRTDLGFLVTRGIPDHRHPSAAVTLAAMPPPHHQHGHQTVVPYPDLMQYAQLLQGGRGGGGHAEAAVQQAHRQQQQQQLMTMMGGRPGVNLPSTFSPSSSASAPQILDFSTQQLIRPGPPSPSPPRAAAMPSSSAAAAPSTPSSTTTASSPSGGAWPYGGERHRNKKDA

SEQ ID NO: 19 - DNA, 585 bp, Zea mays
atgaaccggttttactccctgttacatttgtgtgacgacgacctgacaggccgacacgctcccctctcctgcgtggattcagtgacgcaccgcagggccgtccacgcgccggcgacggagccacagcggcggcctcggtaccgcggcgtgcggcagcggccgtggggcaagtgggcggcggagatccgggacccggtgaaggcggcgcgcgtgtggctcggcaccttcgccaccgccgaggacgccgcgcgcgcctacgacgacgccgcgctccgcttccggggcgccaaggccaaactcaacttccccgctaacgcgggccacggcaccgctgccgccttccagccccgctaccgcggccagcatcggccggggacgccgagcgctgctgctgctcctgttggctgtcacgaggagccgtcgtcgtcgttccccgacctcggccagtacgcgcgcatcctgcagagcggcggcggcgacttggacctgcaggccgccttcgcgggcggggtggcgccggcggggcggtcctcgacttccgcggcgtcggcgtcggcgtcgtcgccgtggcaaggtgctacttga

SEQ ID NO: 20 - protein, 194 aa, Zea mays
MNRFYSLLHLCDDDLTGRHAPLSCVDSVTHRRAVHAPATEPQRRPRYRGVRQRPWGKWAAEIRDPVKAARVWLGTFATAEDAARAYDDAALRFRGAKAKLNFPANAGHGTAAAFQPRYRGQHRPGTPSAAAAPVGCHEEPSSSFPDLGQYARILQSGGGDLDLQAAFAGGVAPAGRSSTSAASASASSPWQGAT

SEQ ID NO: 21 - DNA, 654 bp, Panicum virgatum
atgccggactccgacaacgagtccggcgggccgagcaacgcggagttctcgtcgccgcgggagcaggaccggttcctgccgatcgcgaacgtgagccggatcatgaagaaggcgctcccggcgaacgccaagatctccaaggacgccaaggagacggtgcaggagtgcgtctccgagttcatctccttcatcaccggcgaggcctccgacaagtgccagcgcgagaagcgcaagaccatcaacggcgacgacctcctctgggccatgaccacgctcggcttcgaggactacatcgagccactcaagctctacctccacaagttccgcgagctcgagggcgagaaggtggcctccggcgccgcgggctcctccggctccgcctcgcagccccagagagagacaacgccgtccgcgcacaatggcgccgccggggccgtcggctacggcatgtacggcgccggcgccggggccggcggaggcagcggcatgatcatgatgatggggcagccgatgtacggctccccaccgggcgcgtcggggtacccgcagcccccgcaccaccacatggtgatgggcgctaaaggtggcgcctacggccacggcggcggctcgtcgccatcgctgtcggggctcggcaggcaggacaggctatga

SEQ ID NO: 22 - protein, 217 aa, Panicum virgatum
MPDSDNESGGPSNAEFSSPREQDRFLPIANVSRIMKKALPANAKISKDAKETVQECVSEFISFITGEASDKCQREKRKTINGDDLLWAMTTLGFEDYIEPLKLYLHKFRELEGEKVASGAAGSSGSASQPQRETTPSAHNGAAGAVGYGMYGAGAGAGGGSGMIMMMGQPMYGSPPGASGYPQPPHHHMVMGAKGGAYGHGGGSSPSLSGLGRQDRL

SEQ ID NO: 23 - DNA, 639 bp, Zea mays
atgccggactccgacaacgagtccggcgggccgagcaacgcggagttctcgtcgccgcgggagcaggaccggttcctgccgatcgcgaacgtgagccggatcatgaagaaggcgctcccggccaacgccaagatctccaaggacgccaaggagacggtgcaggagtgcgtgtcggagttcatctccttcatcaccggcgaggcctccgacaagtgccagcgcgagaagcgcaagaccatcaacggcgacgacctactctgggccatgaccacgctcggcttcgaggactacgtcgagccgctcaagctctacctccacaagttccgcgagctcgagggcgagaaggcggccacgacgagcgcctcctccggcccgcagccgccgctgcacagggagacgacgccgtcgtcgtcaacgcacaatggcgcgggcgggcccgtcgggggatacggcatgtacggcggcgcgggcgggggaagcggtatgatcatgatgatgggacagcccatgtacggcggctccccgccggccgcgtcgtccgggtcgtacccgcaccaccagatggccatgggcggaaaaggtggcgcctatggctacggcggaggctcgtcgtcgtcgccgtcagggctcggcaggtag

SEQ ID NO: 24 - protein, 212 aa, Zea mays
MPDSDNESGGPSNAEFSSPREQDRFLPIANVSRIMKKALPANAKISKDAKETVQECVSEFISFITGEASDKCQREKRKTINGDDLLWAMTTLGFEDYVEPLKLYLHKFRELEGEKAATTSASSGPQPPLHRETTPSSSTHNGAGGPVGGYGMYGGAGGGSGMIMMMGQPMYGGSPPAASSGSYPHHQMAMGGKGGAYGYGGGSSSSPSGLGR

SEQ ID NO: 25 - DNA, 618 bp, Zea mays
atgccggactccgacaacgacgagtccggcgggccgagcaacgcggacttctcgtcgccgcgggagcaggaccggttcctgccgatcgcgaacgtgagccggatcatgaagaaggcgctcccggccaacgccaagatatccaaggacggcaaggagacggtgcaggagtgcgtgtccgagttcatctccttcatcaccggtgaggcctccgacaagtgccagcgcgagaagcgcaagaccatcaacggcgacgacctcctctgggccatgaccacgctcggcttcgaggactacgtcgagccgctcaagctctacctccacaagttccgcgagctcgagggcgacaaggcggccgcgggctcgcagccgccgccgccgccgtcgtcaacgcacaatggcgcgggagtgcccgtcggctacggcatgtacggcgccggcggaggcagcggcatgatcatgatgatgggacagcccatgtacccgccggccgcgtcgtcggggtactcgcagcagccgccgcaccaccagatgtccatgggcgggaaaggtggcgcgtatggccactgcgacggctcgtcgtcatcgccgtcagggctccgcaggcacgacgggttgtga

SEQ ID NO: 26 - protein, 205 aa, Zea mays
MPDSDNDESGGPSNADFSSPREQDRFLPIANVSRIMKKALPANAKISKDGKETVQECVSEFISFITGEASDKCQREKRKTINGDDLLWAMTTLGFEDYVEPLKLYLHKFRELEGDKAAAGSQPPPPPSSTHNGAGVPVGYGMYGAGGGSGMIMMMGQPMYPPAASSGYSQQPPHHQMSMGGKGGAYGHCDGSSSSPSGLRRHDGL

SEQ ID NO: 27 - DNA, 543 bp, Zea mays
atggcggacgctccggcgagccctgggggcggaggcgggagccacgagagcgggagccccaggggcggcggaggtggaggcggtggcagcgtcagggagcaggacaggttcctgcccatcgccaacatcagtcgcatcatgaagaaggccatcccggctaacgggaagatcgccaaggacgctaaggagaccgtgcaggagtgcgtctcggagttcatctccttcatcactagcgaggcgagtgacaagtgccagagggagaagcggaagaccatcaatggcgacgacctgctgtgggccatggccacgctggggtttgaggactatattgaacccctcaaggtgtacctgcagaagtacagagaggtgcagggtgatagtaagttaacttcaaaatccagcgatggctccattaaaaaggatgcccttggtcatgtgggagcaagtagctcagctgtacaagggatgggtcagcaaggaacatacaaccaaggaatgggttatatgcaacctcagtaccataacggagatatctcgaactaa

SEQ ID NO: 28 - protein, 180 aa, Zea mays
MADAPASPGGGGGSHESGSPRGGGGGGGGSVREQDRFLPIANISRIMKKAIPANGKIAKDAKETVQECVSEFISFITSEASDKCQREKRKTINGDDLLWAMATLGFEDYIEPLKVYLQKYREVQGDSKLTSKSSDGSIKKDALGHVGASSSAVQGMGQQGTYNQGMGYMQPQYHNGDISN

SEQ ID NO: 29 - DNA, 537 bp, Zea mays
atggcggaagctccggcgagccctggcggcggcggcgggagccacgagagcgggagccccaggggaggcggaggcggtggcagcgtcagggagcaggacaggttcctgcccatcgccaacatcagtcgcatcatgaagaaggccatcccggctaacgggaagatcgccaaggacgctaaggagaccgtgcaggagtgcgtctccgagttcatctccttcatcactagcgaagcgagtgacaagtgccagagggagaagcggaagaccatcaatggcgacgatctgctgtgggccatggccacgctggggtttgaagactacattgaacccctcaaggtgtacctgcagaagtacagagaggtgcagggtgatagcaagttaactgcaaaatctagcgatggctcaattaaaaaggatgcccttggtcatgtgggagcaagtagctcagctgcacaagggatgggccaacagggagcatacaaccaaggaatgggttatatgcaaccccagtaccataacggggatatctcaaactaa

SEQ ID NO: 30 - protein, 178 aa, Zea mays
MAEAPASPGGGGGSHESGSPRGGGGGGSVREQDRFLPIANISRIMKKAIPANGKIAKDAKETVQECVSEFISFITSEASDKCQREKRKTINGDDLLWAMATLGFEDYIEPLKVYLQKYREVQGDSKLTAKSSDGSIKKDALGHVGASSSAAQGMGQQGAYNQGMGYMQPQYHNGDISN

SEQ ID NO: 31 - DNA, 1065 bp, Zea mays
atgtggcccatggaggaggagatgttcctggcggtgaagcggacggagcacgtcgaggtcacttcccgggcgatggattccgcgccggcggcggggcccaggacggtgcgggtcttctgcgacgacaacgacgcgaccgactcgtccggcgacgaggcggagggcgcggccgcgcgcaggagggtgaagcgctacgtgcaggagatccggctgcagcgggcggtgccggtgaaggaggaggcggcgtccgcgagggccgtcgcgggggagcggcccaggccgctggccctcccggggcggaagaggaagtccgacggcgccggcgccggcgccgagccccggttccgcggcgtgcggcggcggccgtggggcaagtacgcggcggagatccgcgacccctggcgccgggtgcgcgtctggctcggcaccttcgacaccgccgaggaggccgccaaggtgtacgactcggccgccatccagctccgcggggccgacgccaccaccaacttcgagcacgcggcggtccccgtccccgtccccgacgaggtcgcgggtcgcctcccgcagccgccgccggctccggcgtccaagaacgcgtcgtcctccgccacgtcctcctacgactccggcgaggagtcccacgccgcggcggcctcgccgacctccgttctccgcacgttccctccgtcctccgcggccgccgacgccacctgcaagaaaccggcgccggccgcggagaccgacgagtcgaccggcgtcggcggcagcaggagcagcgtgtacgcataccccttcttcgccaacgacgactgcttcggcggcgacttccccccgctgtacactgacttcgacctgctggccgacttcccggagccgacgctggacttcctggcggacatccccgaggagaagctgccgccgctgccggagccgttccccgcggcgagccccgacgagtgcccgtccgacgcggaggcggcgtcgccggcgaggtggcagcaggtggacgacttcttccaggacatcaccgacctgttccagatcgacccgctgcccgtcgtctag

SEQ ID NO: 32 - protein, 354 aa, Zea mays
MWPMEEEMFLAVKRTEHVEVTSRAMDSAPAAGPRTVRVFCDDNDATDSSGDEAEGAAARRRVKRYVQEIRLQRAVPVKEEAASARAVAGERPRPLALPGRKRKSDGAGAGAEPRFRGVRRRPWGKYAAEIRDPWRRVRVWLGTFDTAEEAAKVYDSAAIQLRGADATTNFEHAAVPVPVPDEVAGRLPQPPPAPASKNASSSATSSYDSGEESHAAAASPTSVLRTFPPSSAAADATCKKPAPAAETDESTGVGGSRSSVYAYPFFANDDCFGGDFPPLYTDFDLLADFPEPTLDFLADIPEEKLPPLPEPFPAASPDECPSDAEAASPARWQQVDDFFQDITDLFQIDPLPVV

SEQ ID NO: 33 - protein, 326 aa, Panicum virgatum
MCGGAILSDLYSPVRRTVTAGDLWGESGSTKNVKNWKRRSSWKFDEDDDDFEADFEDFNDCSSEEEVDFVREEKEFQLNSSNFVELNGHTTKVASRKRKTQYRGIRRRPWGKWAAEIRDPQKGVRVWLGTFSTAEEAAKAYDVEALRIRGKKAKVNFPNTITAAGKHHRQHVARPAKRMSQESLKSSDASGHVVSAGSSTDGTVVKIELIESPASPLPVSSAWLDAFELNQLGGLRHLEADGRETTEETDHETGVTADMVFGDGKVRLSDDFASYEPYPNFMQLPYLEGNSYENIDTLFNGEAAQDGVNIGGLWNFDDVPMDRGVY

SEQ ID NO: 34 - DNA, 1110 bp, Panicum virgatum
atgtgcggcggtgcgatcctcgccaacctcaccaagcagccgggcccgcgccggctcacggagcgggacctctggcaggagaagaagaagcccaagaggggcgccggcggggggaggcgctggttcctggctgaggaggatgaggacttcgaggccgacttcgaggacttccagggcgactccgatgagtcggatttggaactcggggagggggaggacgacgacgtcgtcgagatcaagcccttcgccgccaagaggacttcctccaaagatggcttaagcaccatgactactgctggttatgatggccctgcagcaaggtcagccaaaaggaagagaaagaatcaatacaggggcatccgccagcgcccttggggtaagtgggctgctgagatcagagatcctcagaagggtgttcgtgtttggcttggtactttcaacagtcctgaggaagctgcaagagcttatgatgctgaagcacgcaggatccgtggtaagaaggccaaggttaacttccctgatgcaccaacagttgctcagaagcgccgtagtgggccagctgctgctaaagcacccaaatcaagtgtggaacagaagcctaccgtcaaaccagcagtgaacaaccttgccaacgcaaatgcatcctacccacctgctgactacacctcaagcaagccatctgttcagcatgccaatatggcatttcatctagcaatgaactctgctagtcctattgaggatccagttatgaatctgcactctgaccagggaagtaactcttttgattgctcagacttgagctgggagaatgataccaagacttcagacataacatccattgctcccatttccaccatagctgaaggtgacgagtctgcatttgtcaacagcaatttgaacaactcactggtgccttctgttatggagaacaatgcagttgatctcactgatgggctgacagatttagaaccgtacatgaggtttcttctggatgatggtgcaagtgagtcaattgataaccttctgaaccttgatggatctgaggatgttatgagcaacatggatctctggagctttgatgacatgcctgctgctggcgatttctattga

SEQ ID NO: 35 - protein, 370 aa, Panicum virgatum
MCGGAILANLTKQPGPRRLTERDLWQEKKKPKRSAGGGRRWFLAEEDEDFEADFEDFQGDSDESDLELGEGEDDDVVEIKPFAAKRTSSKDGLSTMITAGYDGPAARSAKRKRKNQYRGIRQRPWGKWAAEIRDPQKGVRVWLGTFNSPEEAARAYDAEARRIRGKKAKVNFPDAPTVSQKRRSGPAAAKAPKLSVEQKPTVKPAVNNLANANASFYPPADYTSNQQFVQHANMPFHPAMNSASPTEDPVMNLHSDQGSNSFDCSDLSWENDTKTSDITSIAPISTIAEGDESAFVNSNLNNSLVPSVMGNNAVDLTDGLTDLEPYMRFLLDDGASESIDNLLNLDGSEDVMSNMDLWSFDDMPATGDFY

SEQ ID NO: 36 - protein, 319 aa, Panicum virgatum
MCGGAILAELIPSPRRAASKPVTAGHLWPAGSDTKKAGSGRSKRHQLADVDDFEAAFEDFADDFDKEEVEDHHFVFSSKSAFSPAHGVRAATQKRRGRRHFRGIRQRPWGKWAAEIRDPHKGTRVWLGTFNTAEDAARAYDVEARRLRGSKAKVNFPAAGARPRRGNAPRPQRHHAAAQPALLAGEKRKEEEIVVKPEIGASFDFDVGSFFDTAFPAAPPAMENSFAGSTGSESGSPAKKMRYDNDSSSDGMSSGGGSALELADELAFDPFMLLQMPYSGGYESLDGLFAVDAAQDVNNDMNGVSLWSFDEFPDDSAVF

SEQ ID NO: 37 - protein, 247 aa, Panicum virgatum; identical to SEQ ID NO: 12

SEQ ID NO: 38 - protein, 354 aa, Panicum virgatum
MVESDQKYNVQQPSQTEESCRNEVTALKNSNNRNKVFNYPTPTLLRESQPMYLDTAAGSPRSCTSSCRDERARGSEVQELKLSPTHARIDRSSPSDGRRRRQIDRQLTKVDPRRHGKRPLPADEEEEEPPPPPPAKLEQLDVEEQYHVSQLQGATFISGSGGGGSSSSPAGAAGPSPEAYAQYYYSARADHDATAVASALAHVIRASPDQLPPQAFYAAGGAPGHQQGDHQQAAPHHHAAAAAEEEQGRRRHYRGVRQRPWGKWAAEIRDPKKAARVWLGTFDTAEDAAIAYDEAALRFKGTKAKLNFPERVQGRTDLGFLVTRGVPDRHHHQGAAVTAPRRPPRPSRRRRRSS

SEQ ID NO: 39 - protein, 362 aa, Panicum virgatum
MCHAAVADSGEQHGRRLLAAGDGGGGDRRQQQQQPQPLEPVVMEANTAASPALSRGRQAREMSAMVAALARVVAGSAPPAKAPPQAVQDASAEEAWWPYDELAAEPSPAFVLDGYSETQPLPEHYWPSAAAATEAATSSQTHYRAASAAAAEEEVPSPSSASAAAGASSSGSAATRKRYRGVRQRPWGKWAAEIRDPHKAARVWLGTFDTAEAAARAYDGAALRFRGSRARLNFPESATLPSPPPPDPASRALPPPPPRPDALLESQAQAPSTGGGMEQYAEYARLLQSAGGDPGGSSGTPSGTLPPPPPPAAYSFAAQGVTPFSYLSPPQSRGEPAGNPAAAWAASHYHGSYPPWRWDHSG

SEQ ID NO: 40 - DNA, 708 bp, Zea mays
atggagggccactactcgcccacggcctccgccgccgaggcgggcggcgagcggcggttccgcggcgtgcggcagcggccgtggggcaagtgggcggcggagatccgcgacccgcacaaggcggcccgcgtctggctcggcacgttcgacaccgcggaggccgcggcgcgcgcctacgacgaggccgcgctccgcttccgcggcagccgcgccaagctcaacttccccgaggacgcgcgcctcaccacggcgccatccgctgcggtcgctccggcggcggcgggatccacgacgacgatggccgtggcggcctcggcgggcgggtcgtaccagcaggaggccagcgccgccgtcgtctctgcagactacctgcagtaccagatgatgcttctgcagggggccaccagcggccaaggcagcagcagtcatggcgggtatcctctgtactacgactatggcggccacggcggggctggcgctggcgctggtggtggtgccatgagcagctcctactctttccccgcctccacggttaccgtggcttctgtgccgtcgtcctctgcctcctccgctcccagctatggcgaggcggcgcagtgggcaggcagctggccggagagtagtgcgtggagctatccggcgaccacggcttcttggtctggatcaagccagtatgagtatccaccgtcaactcgtccacctcaatag

SEQ ID NO: 41 - protein, 235 aa, Zea mays
MEGHYSPTASAAEAGGERRFRGVRQRPWGKWAAEIRDPHKAARVWLGTFDTAEAAARAYDEAALRFRGSRAKLNFPEDARLTTAPSAAVAPAAAGSTTTMAVAASAGGSYQQEASAAVVSADYLQYQMMLLQGATSGQGSSSHGGYPLYYDYGGHGGAGAGAGGGAMSSSYSFPASTVTVASVPSSSASSAPSYGEAAQWAGSWPESSAWSYPATTASWSGSSQYEYPPSTRPPQ

SEQ ID NO: 42 - DNA, 894 bp, Zea mays
atgcatggcgcgcgcggaagccaacgcaggggtcaaaagcgcgacgccgccgacgcgcgctatatatccgtcgcattgcaacctaacgctacgtcgtgtgcgcgagtagtagtgctgtgctgtgtgcctgtgtggctgtgtgctcatctcgatctacatatcgtgtccacgcacgcaggaggagagcgggcggtccacgacgacgtgcagcgcgcggcggccatggagggccactactcgcccacggccgcccccgaggtgagcggcgagcggcggttccgcggcgtgcggcagcggccgtggggcaagtgggcggctgagatccgggacccgcacaaagcggcccgcgtctggctcggcacgttcgagaccgccgaggccgcggcgcgtgcctacgacgaggccgcgctccgcttccgcggcagccgcgccaagcttaacttccccgaggacgcgcgcctcaccacgccgtcgtccgccgcggccacggccgccaccgcggcggcgggatccacgaggacgatggccgtggcctccacgggagggtacccggccagcgccgcctctgctgactacctgcagtaccagatgtttctgcagggtccgcagggagccaccagcggcagtcatggaggggggtatcctctgtactacgactatggcggccacagcggggttggcgatggttccgtgagcagttcttcgggctcctactcttttccggcctccacggttaccgtggcttctgtgccctcctctgctccgagctacgacgaggcagcgcagtggacaagctggccggagagtagtgctagtgcgtggagctatccggcgaccacgggttcttggtctgcttcaagccagtatccaccgtcaactcgtccacctcagtag

SEQ ID NO: 43 - protein, 297 aa, Zea mays
MHGARGSQRRGQKRDAADARYISVALQPNATSCARVVVLCCVPVWLCAHLDLHIVSTHAGGERAVHDDVQRAAAMEGHYSPTAAPEVSGERRFRGVRQRPWGKWAAEIRDPHKAARVWLGTFETAEAAARAYDEAALRFRGSRAKLNFPEDARLTTPSSAAATAATAAAGSTRTMAVASTGGYPASAASADYLQYQMFLQGPQGATSGSHGGGYPLYYDYGGHSGVGDGSVSSSSGSYSFPASTVTVASVPSSAPSYDEAAQWTSWPESSASAWSYPATTGSWSASSQYPPSTRPPQ

SEQ ID NO: 44 - protein, 217 aa, Panicum virgatum
MPDSDKESGWPSNAEFSSPREQDRFLPIANVSRIMKMALPANAKISKDAKETVQECVSEFISFITGEASDKCQREKRKTINGDDLLWAMTTLGFEDYIEPLKLYLHKFRELEGEKVASGAAGSSGSGSQPQRETTPSAHNGAGGAVGYGIYGAGAGAGGGSGMIMMMGQPMYNSPPGASGYPQPPHHQMVMAAKGGAYGHGGGSSPSPPGLGRQDRL

SEQ ID NO: 45 - protein, 209 aa, Panicum virgatum
MPDSDNDSGGPSNAGGELSSPREQDRFLPIANVSRIMKKALPANAKISKDAKETVQECVSEFISFITGEASDKCQREKRKTINGDDLLWAMTTLGFEDYVEPLKHYLHKFREIEGERAAASSGASGSAAAQQQQGDVARGAANAAGYGAPGAGGMMMMMGQPMYGSPQQQPPPQQQQQQHQQHHMASGRQRWLRPSRRRRRLVVVVGAW

SEQ ID NO: 46 - protein, 180 aa, Panicum virgatum
MADAPASPGGGGGSHESGSPRGGAGGGGGGVREQDRFLPIANISRIMKKAIPANGKIAKDAKETVQECVSEFISFITSEASDKCQREKRKTINGDDLLWAMATLGFEDYIEPLKVYLQKYREMEGDSKLTAKTGDGSIKKDALGHGGASSSATQGMGQQGAYNQGMGYMQPQYHNGDISN

SEQ ID NO: 47 - DNA, 495 bp, Zea mays
atggccgacgacggcgggagccacgagggcagcggcggcggcggaggcgtccgggagcaggaccggttcctgcccatcgccaacatcagccggatcatgaagaaggccgtcccggccaacggcaagatcgccaaggacgctaaggagaccctgcaggagtgcgtctccgagttcatatcattcgtgaccagcgaggccagcgacaaatgccagaaggagaaacgaaagacaatcaacggggacgatttgctctgggcgatggccactttaggattcgaggagtacgtcgagcctctcaagatttacctacaaaagtacaaagagatggagggtgatagcaagctgtctacaaaggctggcgagggctctgtaaagaaggatgcaattagtccccatggtggcaccagtagctcaagtaatcagttggttcagcatggagtctacaaccaagggatgggctatatgcagccacagtaccacaatggggaaacctaa

SEQ ID NO: 48 - protein, 164 aa, Zea mays
MADDGGSHEGSGGGGGVREQDRFLPIANISRIMKKAVPANGKIAKDAKETLQECVSEFISFVTSEASDKCQKEKRKTINGDDLLWAMATLGFEEYVEPLKIYLQKYKEMEGDSKLSTKAGEGSVKKDAISPHGGTSSSSNQLVQHGVYNQGMGYMQPQYHNGET

SEQ ID NO: 49 - protein, 161 aa, Panicum virgatum
MADDGGSHEGGGGVREQDRFLPIANISRIMKKAVPANGKIAKDAKETLQECVSEFISFVTSEASDKCQKEKRKTINGDDLLWAMATLGFEEYVEPLKMYLHKYREMEGDSKLSTKAGEGSVKKDAISPHGGTSSSSNQLVQHGVYNQGMGYMQPQYHNGDT

SEQ ID NO: 50 - protein, 127 aa, Panicum virgatum
MADAPASPGGGGGSHESGSPKGGGGGGGGGVREQDRFLPIANISRIMKKAIPANGKIAKDAKETVQECVSEFISFITSEASDKCQREKRKTINGDDLLWAMATLGFEDYIEPLKVYLQKYREVTKHL

SEQ ID NO: 51 - protein, 369 aa, Panicum virgatum
MCGGAILANLTKQPGPRRLTERDLWQEKKKPKRGAGGGRRWFLAEEDEDFEADFEDFQGDSDESDLELGEGEDDDVVEIKPFAAKRTSSKDGLSTMTTAGYDGPAARSAKRKRKNQYRGIRQRPWGKWAAEIRDPQKGVRVWLGTFNSPEEAARAYDAEARRIRGKKAKVNFPDAPTVAQKRRSGPAAAKAPKSSVEQKPTVKPAVNNLANANASYPPADYTSSKPSVQHANMAFHLAMNSASPIEDPVMNLHSDQGSNSFDCSDLSWENDTKTSDITSIAPISTIAEGDESAFVNSNLNNSLVPSVMENNAVDLTDGLTDLEPYMRFLLDDGASESIDNLLNLDGSEDVMSNMDLWSFDDMPAAGDFY

SEQ ID NO: 52 - DNA, 1113 bp, Zea mays
tagtttcttctgtgcgttttatggatgaaatatcttatgatggtattgtttgccaaacctatgacaccgacaaaattacatttactagtcatatgaccaaaatttgatcagatggacttgtgtacttttcgatggcaaacaattaattaaagagaatgaatacatagtttctctataaaacacatatttggcatgaaatttaatagtttagatctatttgcatattcacaatgcatgaaacacctaatattttcatagtaccagttacaattatgttttactatatattaataaatatcgtagaagttgtttttccctaaaaccctaatattccaaatgagactttggagccttggacaccaaagtataaatgtctaataacctcaagtacaaatgtgatgttagagtttttggattccccttattaaattttatgacgtgtcactcaaatgattgaatatcaattacgagtattaaatgcaggcttaataaaatcatcttattgttagttttcatatgtgtaattagttttatataattaaaatatatttaatattcggaattatcatttaaagtgacacaaaccaaacttttacatcattagaaccaaacacactgttaatgactaatatcccagattgagattagatttataacatacttgtgcaaggacgtctctatcagatcgtgtatatggtcgtacaaaatattgttttgcattatagataactattatagatatctttgtttatagagtagaatttcaaatggtggatcatgagatacgagcagatagcataaaactctatatctacgatggaatgtcctagtcctagctatctcctgcctactttgcaccattattactgtagcttttccgtttccaaccccgtggcttcagcttgacgcatctgtccactgaggtgtggacccacctcccttccctactcgcctgcagactgtgccagtggcggccagacaataaatagggcccacaccccgtccgcgtccgcgctctccaaagtttcgtccaccatttcgctcccgcaagcaccgatttgtttactgctctctccgacgcgcggcggcggagctccctacgacgactgaacc

SEQ ID NO: 53 - DNA, 1452 bp, Zea mays
cagtccacaggccattccagtatgcctcctctgtcgccagaacatattcacgccagccaagatcagtttccatagtactgcatttatggcagtagcatagataagtgtatctccaagatctgataaaaaaaacgattttgagattttttttccagattttaaacgagaagtgtggggggggggggggggggggaattcacagcgcaggggtttggtgggcgggttggctccctttgccttggcacgcaaggagaccgcttgtctgacgttttgctgccccgggccgactgaaaaagataacgatcagattattaacagttcacgagcaagtgaaaagaaaaagagagaggaccccggcggaaatatctccaacgaaaaaactacgcgtggcatcagcatccctcgtgaatcccgaaattactgcacgactggatcacttaacagcacaggagtggccggggtgaaaaaaaagggagagaaaaaaaaaagagagagagagagaaaggaagccgctgcacgtccgtccgtcggcacgccgcgcggcctttcaccacccccaaagatttgaccacagcttaaaccccaccccgaccccgtgcaagtgcaaggccgtgctcacgtgacgccccctgacacgtggggccgccggaagcgcagctcccccgacgcgtcggccgccgctagaatctccgcgtcgcttgcaggtgggaccggaggaggatacggcggcccggtaccgttggatacccggctcagctgtcatttacaggtgggtcctgcgcaggggaattactgcctgcctgcgtgtttgcctgtgcggtgggaaaagtaaacgtacgggggcaggtggttaggatgcgtacgtcgatctgtttttactgcctgcggttaaacaggcagttctgccagccagcgagcagccagggcattcactttttcgtcgttttttcaccggctcgtctgctagaccacacgctttcgaaatttgaaataacgtcctttttgaggggaatttctgtgaaaagggccggagtacgagaaacacccccaccccaacccggaggaaaggaaaggcgagggaagaaaaaaagaattttggaggtgtaaacaaaaaccccgggctttccgaaatgggctccgggatgctataaaagtggggcggactcccggtgctcagcctaccagcacagcagcagcaacaacaacaaacagcgacagcgagcgcatcaaagcacccacgcagggcagcaggccaggcggcggtgctgccggcagagacacccacccccaccacacccaagcccggtgcctcgcgtctccactccacctccggggcgtaggaggtcgggtgcccgtgccatcccccacataccaaagcgtcggaagagggtttcttgaggggttttagtcgggtgtccaagcttggttgcggagtcaaatgcgagcggagtg

SEQ ID NO: 54 - DNA, 1051 bp, Zea mays
acatctcatcctggcgcgtgtatgacacattctttctctctctcaactttataattaaaacacacataagtcatatatttaagctgagtcaatatttaatcaaaatcacaaattctggaattaatactttaatgtcctaacatttaagatgtttcttagattattactgcctctaatttaattaaatggtcaagcccaattaaaatccaataatcccaccgaattctaaggcacataccaaatacttgtaaggaacagtgaaaaaaggaccgaaggagagtaaattaggcaatctaattttcctctttaaactatagcctctatttttattctaattaaaacgtatgcaataaataaactatcttatttgctactatagttttaagtattgttatctctacccaaaaagtagttaggcaacctcgattctaaacctatccattagcatatgagtaagctaaaagttaaagcatataaaaggtaacacaatataaatacagaagtttaaatacaatagatacaaattttcattaacacatcagtatttttattgaggtatggaatacacaccaaggctcaaacttcctatcatgtaaatttgtggatagcttaaggccttctccctatgcaaatgatgctttgattgtgacctctcttggatggtccctgccctcgagtttacgttcgaaacactagacctcgtttcttttgtacacagtgatggtcacaccataaatataaagtttttaaataaactagtaccgaaggtggatgcaaattagaggaagaaatacatagcatctatatatggtgaactttgttgaccctctagcataatactacggataccaaaattcaaaccttgtgatgaggcatgcatgcttattctccgtaaaccaagtaacgtaaaattcaggcaaacaaacagtggaggagctattagtacggagggtgtctgaaacagttgttcgtagacgatttgactgagtatttaaatagaataaaataagatggtacaagaagggaaattaaagaccttttcaacgcaccg

SEQ ID NO: 55 - DNA, 1380 bp, Zea mays
atcacaaccaacggttcatatcgctagataaacctctctcccttacacacttaggtgttgtttggttccatgcgaggagcggtaatgtcaatgggagccgttgtcgatgctatgcattgcatattaacaactaacacgctttgtttggtggtcggttgggaacaaaggttacaatgagagatgggacgggatcgaataacttcgggtgggtcgcggtaacaagattgtactactctgtatctgatagcgccgcgttccgctgcaggctcggtcaaccaaacgatatgtaaccggaccgggtcacaccgtaaatcgatcgcgggggtaaataacgcaaacccaacatcaccttaaatccaatgaataatattatttagatcattcatcctcctcctccacccacgaccacgacgtcccctcccctcactcgtttcgtcgccgttgtcaactcatcctcttctttccttgggatcatagcattaacatgagctatatatattagttagcagtaattgttgatccaaaagtagaggctccactaaaagccctctgctcagtaaaatattacttcattcattttgaactataataagaatatttaaattttctgtggtgtaagtcactacaggttcaggttcgtcggattcattttgatgaatgtcatgaattcactttcactatgtgtcaagtcacctctcactaccttccacgaacgcgtggtcggcgtgttggcaagcccgctcgggaggagcctctgcccctgcattcaaggcctgctcgacgcaggtccctatcgggttttgcttgtctgtcctagcttgtatttgcatacgtggtgtaggagtatgagttgtttgtggttaagttcgaatagatacgcggctagcaactcagttgctgcagtagctgtagcctgtagcgccatagacacgtcacacaagcggtcaaaagacccaggacacgtcaggctcagtccctccacgtcctcactcctacgtggaagccacccaggggcccaccccgtggatcacgcacctgatcccgcgtgccctggcccacgcggtgggcggacggccccggccccgccagcgtggcggaaacggacggcatcggtggatgccggctggcccccagacttatcgacaaacaaacggccggggccgcgacacgtcgccccctggatgcgttcttatccccacgatgcatccgacgaccgctgccccattctccactccgctctctcaaactccccggctccccgctctcccccgcccgtccagctcgtgctccgcctccgctgctctgccctcttcctcctctgcgtttctcctcagagctgtttgacttgaccggacagtgctgttcggtggctcggccgcg

SEQ ID NO: 56 - DNA, 1381 bp, Zea mays
cgctccgagagagcctgaccatctgagaacacattggtcaccaaaagcaccaccaaccggcctagacaaagcagctcagttgacccccgcctcgacatcttcgatggccggcatcacctttctccccttctttttattcttcgctgtcttcaccttgtcttgatttaacagctccatgattgcatccatttgcttcttggagagaggctttgtgagaaggcttgtcatctgctcaaatgactcatcaaagttagtacattttgaagaactaattattattatatagaatgcactgcacatatattactattaccagttttcttgggcacagcagaaaacatgcacacgcagatagaaaaaggagaggccataaaccaaaaggctttaagaatatatgtaaagatatgtctaaatatatggctatatctggttaagcaagataacagggctctggtcatcagtagtagtggccttttgcccttgcccctctctctcacctctcttttctcagccttgcttccgatggatcccatcccactgccatcctttctttcccttgcgcgcattgcctagccggccggccggcctgctattaaaccactttacccgccccctctcgctcacgctcgacgcagctcccttttccttgtttgcttattgcaagtctctgcaagaacctgctagagaggaacaaggtagagtagtatcgcttttttccatctaggttatctctttttacatgaaaaatttcagccgtatttcgttctccatcagtcctgcgataatatatacgcgcgtcttgtgtgatccggcatatgtatagttcctgctaactgatcgagatcgctctcgtttgtactttctccctttgaggaaagagtttccccttttctgtgcttcaagttcttgtaaggaaaaccatgcctgccagcttcttctgctacttgtatgatgattcttatttgcttattacttgatttccgttttttttcttgctttctatatgtatgtatctgggctgtcttcccctgcgtctcgttactgctaagctttggaaggtttcaactctttgtatacgatgaggtttctgctcctagtagcagatccgcgcatatgactagatgtttgaggaaaagaaaagggcaagacgctatatatatatgcagcacgcagtcgcacatatattcagttttccaatctgcctcttgctttatgataattcaacttgcgctgattatattcttggctacctagaaatgtctaattaaactttgtttgctagctagattttgttgcttcctttcgcatctgatctttttatctcttctgagtgctccgcaaagccttccagtgttgaagaagctgctggaagaacgaagag

SEQ ID NO: 57 - DNA, 1619 bp, Zea mays
ttttaagttaactgggtgaatttcgagatcaatttagttttatatctcattctttagttgagctcgtgcaaaccggttgaaccggttttacctagttcgtttctagtttttgttaaaaaagttttcgcttgtctattcaccctctataggcaactttcaattatgtaatcactttttttttcttttttctgtttaaaatctcagtttcaaacttccaattgattttgaatacgaggtttgggtttaaattcatattggaggcaaaaatcgaaagttccacgtgatgctaggttttatttcggttttctatctcctattgtttttcacgtttcaacttgattcaaattctagttttttttaacttaagcacaattaaatacaacataaaaacaacatggattcaagttctatttcaatttttattaactattatgttgtctagtctgttcaagcacataatacttataaatataaaattaaacgaaatcacatatttccacaaatcttgggtactacactcggagacgacgatggattccatctcaatttggatgttgattatagctctatttcagttgtcactgttgtcctaacacgccctattgtgcatgatagtgcacgtgctcaacgtaaaagaaaagagatcagtaacaagtagcagcactgtacaaggtaagccgtgattcaattaaaactgtttgagcaattcagttgctagatcgttccaccatcgataattcgatatgtacgatgatataaaaagagcccataagtttgtcttgaaaaggttgatcaaataatttaaattagatgataaaaaacatggaagatgtgggagtggacgacggctatgaagaatagtactatatcaggtttatacgtaaaatttatttttgaaatgtttttataatctgtttgaattgtattttttgcttaattatgtgattggatgttttttcatgaaatgtcgagttttattttaaataaaattctgtaaagagaagttgctgcgctgagaaaactataaatcgatagtaaaggctgtacgcaacgtttaagtccttgtttgaatgcgtatgaatctgagaaagttcagaatgattaaatcttttttatttaattttaatttgagagagattaagttctctccaattctctttaatttagacgtaatcgaacaagctggttgccaaactagatgagtacattttgtccactgccatagagccatcgactacaaaagtctagaacacagtggaaagcaccagacaacgcgcgaccaaaagggcccaggccccagcgccccagtccgggggttgtgttcgccgacctgtgcgtgcctgctcgtcacgtcacgtccctatttgcccgtcttcctcccctccagacccttctcgaacgccccttcgttctggatccaacggtcggtctctgccgggctcgaacgttctcgaaaccacgtcacccccgataaaaccccacgcacagcctcctcccttcctcaaccatcattgcaaaagcgaagcaagcaatccgaattctctgcgatttctctagatctcgaccacccctactagttttggttcctcctttcgttcgagagagcgtttctagtggca

SEQ ID NO: 58 - DNA, 1244 bp, Zea mays
tgacaactgatcagcctccttgagaagttgcttgatttcaagccgcactttgatctgctcatcactaagtcctccgctctggatgacaaaagcacagaacgcatgagtggcaagtggaaacactagagcgaaataaatacaaaaccgcagactacaggctaacagatagggagaccgggaagacaaagactcgagcctgcattcaacagttacagtcgcctcggccaaaggttgagaaatttgcatcaaaatccaaactgtctagggccatgggaaatagttcctcggaatcagagttcaattcatggacgaaatagatggaactgatggtaggctactcttccgcccaatcagaattcacggaagatccaggtctcgagactaggagacggatgggaggcgcaacgcgcgatggggaggggggcggcgctgacctttctggcgaggtcgaggtagcggtagagcagctgcagcgcggacacgatgaggaagacgaagatagccgccagggacatggtcgccggcggcggcggagcgaggctgagccggtctctccggcctccgatcggcgttaagttggggatcgtaacgtgacgtgtctcctctccacagatcgacacaaccggcctactcgggtgcacgacgccgcgacaagggtgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagtatcagatcaaccggccaataggacgccagcgagcccaacacctagcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccaacactttcacgctcagcttcagca

SEQ ID NO: 59 - DNA, 1299 bp, Zea mays
atacaataataaaatcatcatatatttaaataaaacactagcaagtctaataacatatgactatagaatcaagatgtgtatgatgacatgacacttgcaattttatcatctcctactactcgacatagtcaatataattgatgtcctccttatctttaaagtttccatgcgaattataaatatatgtatgaagagtaatgattgataagaaactataaataagagtcacaatagttcaaacaactctaaactatatatcattagatagatcttgattttagaaaaataacgaaatcagtttcataattttctaagttaagatgaatttacaaagattagtttagatttaatattttttctgaaaaaataccgatttcggaaacgggcaaaagagatccaaactatttctgtttttttttaccgatttcatttccgtattttcggtaacggtttccggtttcgtatgaccctaaattttggtaaagtttcgaaaaaaaatattttaagaactgaaaattaacgttcctgttttcatccatactaatggctctttaccgctaaaatgttgcccacaatcattgagtaggtttagacgtgagagcaaacagtacaacattacgattcgcccttgcccaaatttacatgccttttccctacggaaacaacatagaatcaagttgacggggttacttacattgaagtggccaaactgatggtagctgtagatttggatgtatgttttctataaattagtcaaaattgagacaaaataaactgcaatttaaaactgaggaaatagtaaaaaaaaggtgaagaagggaggaagaggaaatcagaagcaaaaaatgggcaactttaggcccattatctcgatggtctcgtcggagtccagatatgtgattgacggattggattgggccgtacatcttgcatgagagttcgccaagatttcattgtttaacaagaagcgcgtgacaacaaaaccaagcctatctcatccactctttttttcccttcccacaatggcaagtggcagctcctgattcgctctggccattcctacgtggcacacaccaggattcttgtgtgataggccactgggtcccacccaccaggtgccacatcagacgccaagccatcccggcagaaccaatcccagcccagcaacagatggtctgctatccagttccaactgtataaaagcagctgctgtgttctgttaatggcacagccatcacacgcacgcatacacagcacagagtgaggtaagcatccgaaaaaagctgtgatctgatcgac

SEQ ID NO: 60 - DNA, 1339 bp, Zea mays
agggcaagttgcaaacaggtgttgatctaaaaaggaagtagtagggaaatgtgaagtgtctttgcgaggaattggaaaatgaagatcacattttctttgggtgcatcatgggaagaaccatttgggactcttttaaggaggcctaagaatgccataaagtttgcaagatctttttgaagagtgtctacctataaacaatagtaaatatcatgtcaaaattttcatcttcgccattattctttaggagaatttagaatgttccgaataaaatatggatagaaaagaagttcccaaagtcatccaattttctacaaaatcttcaactttaagattgagagtgggtgttgtaaagttcttggaagatgagttgaaccccatggaggcgttggctaaagtactgaaagcaatctaaagacatggaggtggaaggcctgacgtagatagagaagatgctcttagctttcattgtctttcttttgtagtcatctgatttacctctctcgtttatacaactggttttttaaacactccttaacttttcaaattgtctctttctttaccctagactagataattttaatggtgattttgctaatgtggcgccatgttagatagaggtaaaatgaactagttaaaagctcagagtgataaatcaggctctcaaaaattcataaactgttttttaaatatccaaatatttttacatggaaaataataaaatttagtttagtattaaaaaattcagttgaatatagttttgtcttcaaaaattatgaaactgatcttaattatttttccttaaaaccgtgctctatctttgatgtctagtttgagacgattatataattttttttgtgcttaactacgacgagctgaagtacgtagaaatactagtggagtcgtgccgcgtgtgcctgtagccactcgtacgctacagcccaagcgctagagcccaagaggccggaggtggaaggcgtcgcggcactatagccactcgccgcaagagcccaagaggccggagctggaaggatgagggtctgggtgttcacgaattgcctggaggcaggaggctcgtcgtccggagccacaggcgtggagacgtccgggataaggtgagcagccgctgcgataggggcgcgtgtgaaccccgtcgcgccccacggatggtataagaataaaggcattccgcgtgcaggattcacccgttcgcctctcaccttttcgctgtactcactcgccacacacaccccctctccagctccgttggagctccggacagcagcaggcgcggggcggtcacgtagtaagcagctctcggctccctctccccttgctccat

SEQ ID NO: 61 - DNA, 2073 bp, Zea mays
attagtcaaactagtaagttataataaatattgttagaacttacaagtgtcattctcacttctcaaaaagttaaatacttttatcatttttatattgcataacaagtatatagtcaattaattgttgaaattttctgtaataaatttatttagagatataaatattgcttgtattttttttataaatctaactaaagttagaaaaatttcatcagtacctactcaaaacaacacttacttttgggacaaacgaagtatatctctaactaaatatactaaaaatatgttttaaattaatttaatagctctaaactctaatgctcaattgactgattcataatggtatcgcggtaaaacagatctatctctatctggatgctctgatgctccattgactgattcataatggtatcgtggtaaaatggatctatctctatctggatgctcatgcaagcagaaatgagaggatcagcagctgatatgaagcggattgaccggtttgcttgggagggagtaccagatttattatacattcacgatgacttgagaccttttttttttgcgatctaggcacgataagaacaatgttggacacaacttaagtctgttttacaacaatgtctctcaaaactatagttttacaatattatactttgcaattatcatgacaataatgtagtttcggtagctccaaaaatacagtagttttgagaaacattgtttagatacaatattataaatcatgtattagacaaaagatagccatgccattaaaactttgaattggactgtagttttttcaatactccaaaaatattatggtacctagaatacgatgtctagaaaacatattttttaaaatgcaaccaaacatcatatgacataaataatatagtatttttttgaaaaccatggtattacctaaaaactacagaatacttcattctgaaataggtcctaacaagttgcagcagctaggtcgtacatcagcaaatagctacttcatcaatctcagaataaacatattttatagatgagttaaactaaaaatatagaagaacaacgtacacgcgttgaatcacaacgtagcgcgatatccattcaactttttggaagtttttactgagcacaaattcgaaaatgggaagcgccacgtaacacgagcgctgggccaatttctgccagtgccagttatcccggcccacatccaatcctggggaagacgcgaacccggctccgcggcacgagttgtccgcacgtacggcacgtcggggctggctcgtccgcccgcgagtgggaggccactgtttcctctgcctcaccgggtcgtgtggcggaggggcgtggggccatggttcgcagcgcggggcgacgagcgcgctcctcctctcgcgcagcgccagcgccaccccgcaccgtggctttatatacacccctcctcccaaccctaccgaatcatcactaccaccgctctctcttcctctcctccatctctcaacgcctgaagctcaccgcacctcccctcctcgccgcggatcccccactactccggtaaccgtctctccattcaccctgcctgctgtctcgctagaatcgcctgcctctgccagcgccgtgacgcgggggcgcggtatggctctcccagatccgcctggcattgctcgctcgggtcgtgccaggccgatctgatctcgcatttgctgcgcgctcctcctgctgcggatcccaccggatctcgctggaatcggagcgcgcgtctctttgaaatgccgcagatctgcgtgcttgcgcgcgtgatctaagtccgggcctttcgttaacgaaatggtccgatctgtggtttggtggaggcaatgccatggtttttccccgtgaattttttttgctgattttaggagcttttttctactgtcctatgttagtaggacaaaaaaaaagaaacatagattagcttcaataggcgccttttagaacagattctgtacagcaactcgtggaaacaaatctgcttccttaatgatgttgcttgttttaacaaatgcggcatcgggcgagcttttctgtaggtagaaa

SEQ ID NO: 62 - DNA, 1200 bp, Zea mays
taatatgccagaccggctcatatacatacaacagtaatacatcaacaaaacgtataaaatatatatatgaccaaaataaaactaagatgttttgtggatgcacattataaacctttggtcagaaagaaaaaaatattacaactagctcacaaaaaatatccagttctctgtttagtgtttaattgagtactatacatccatacagaataaatatacaatgatcatcatcactattcactatccatatctaggtattggttctcgatggcttattaaagctctagattctccaagttatgctagtcatgtgggctttgacagaccttagttaaatactgagtctatattttgtgggccttagttaaatgggtcgtggcaggccggcccgtgggcttgacttgaggcccaggcacggcccacaatgtgggccgtgccggcccatgcccacaattaggttgggcagtgccagatatgggccgtgccagaaattgtgtgctttgggccggcctattaggcacaacataaatgtacacctatagccgcatagccgctggatgtgagatgaatgtctcagatttaaaatgtgcacttgagcaccgtacctctttgaacaacagatatgttcctttaagattgatggtggaaaaaaattagtcagtacctcactgtatggcggcattgtttgattatttcagttcgcacccgttggaccttgctcattaaaaaagtttataccatggagtctttgcatgtagttgtgtagtaggggaagagtggcataggaggaatcacaacttcagctagcttctctagccttagggtatttttgtctttttgcagttcggtcttttcgcagccctgcgctgccccccctgtccgcctgtccctagacctgttttgcgtcggcggggaagacagttgacaggaaggacacgatcttcgtgtccgatgccgatcttcatgcgagcagcgagccactacgttgcgctgccagtgtcggctatggtatccaggcattcgttgtgcacgttgacgatgagctcgaagccggtccgggtgaacgcgagcagcacggtgaggtcaacgtcgtacatccgcacgtcgatgctgaggccagccagcagcggcatgacagattgcggcgtcaggagattgtgccagtaggtggcggggctgggggcagaccggcaggcgaggcct

SEQ ID NO: 63 - DNA, 1215 bp, Zea mays
ctacagaggtcgaacgtgatttggaaacatagctctattgttctctatctcatgcataaatatggtgcaatgaagaatattagggttatgatgtcgaaatctcactcgaactcgtgcctcatcataaatagcacactatcaattgttctatggctgttcaaatagggacaatcttgaaacaacatttctcacatgtaaaacgttgtgaagtatgccaactgaaacggatgacacatacacttcgtgaaccaatcgatattttacttgcttctatgttaaataatgttataatacaatattttattcaaatgctaaaacttattactagataaaaataaaatttaattatcttcaaaaactaaccaatagatattccatcataactacatttaccaaactaatatactaaaaaatataggataattactaaattaatcgtgcaataatcagtatttatgagattgataattttaaattttgtgggctacaaacaaaaattaaaacttacttttcaagttggagataagaacaatggtagacgtagctcgggatggtatggcgtcggtgcagacggttaccctttgtgcgaagtggcgcgggcacgagggtggggacttggtacatgcatgagagagaggaagaacgaaacaacttctcaaattaaagcatatgaaaatcacctaatttttgtctgtcggtggaaactaataactagtttttattatcttttttaataaggatccacgaaaattatttttgaccgatgaaaatcctggatcttcgtattatgtttcgccttttcccgactctttgcatgctagatttccatgcttggactaaaacgaagataataaaaccaatctatcattttcacacgatgtattcatacttgcaatagataaaccactactccgacgggatttgctttctgacctctgaaatcttggaaggattatgtgtctacacttctcgatcgaggggaaaaagtcgtagtaccaagttgtagttaaatttgtttcttcgatgacaaaacaaaggagaggggcccgcgcggcgcagcgcagcgcagttggctggttccggaacacgaaaaccaagcacactccaccagctgccatccaccgggttggatggagattacaatactcgaatagtcagccagccagccggcttgaacgtgcagttttcccctataaaacggccccgacccggccg

SEQ ID NO: 64 - DNA, 1694 bp, Zea mays
cacggaagatccaggtctcgagactaggagacggatgggaggcgcaacgcgcgatggggaggggggcggcgctgacctttctggcgaggtcgaggtagcgatcgagcagctgcagcgcggacacgatgaggaagacgaagatagccgccatggacatgttcgccagcggcggcggagcgaggctgagccggtctctccggcctccggtcggcgttaagttggggatcgtaacgtgacgtgtctcgtctccacggatcgacacaaccggcctactcgggtgcacgacgccgcgataagggcgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagaatcagatcaaccggccaataggacgccagcgagcccaacacctggcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccagcactttcacgctcagcttcagcaagatctaccgtcttcggtacgcgctcactccgccctctgcctttgttactgccacgtttctctgaatgctctcttgtatggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcatacggtatttatttaagcacctgttgctgctatagggcacttgtattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgacgataagtcaagatcagatgcacttgttttaaatattgttgtctgaagaaataagtactgacagttttttgatgcattgatctgcttgtttgttgtaacaaaattttaaaataaagagttccctttttgttgctctccttacctcctgatggtatctagtatctaccaactgatactatattgcttctctttacatacgtatcttgctcgatgccttctcctagtgttgaccagtgttactcacatagtctttgctcatttcattgtaatgcagataccaagcgg

SEQ ID NO: 65 - DNA, 1507 bp, Zea mays
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 66 - DNA, 2833 bp, Artificial Sequence
  pYTEN26
cacggaagatccaggtctcgagactaggagacggatgggaggcgcaacgcgcgatggggaggggggcggcgctgacctttctggcgaggtcgaggtagcgatcgagcagctgcagcgcggacacgatgaggaagacgaagatagccgccatggacatgttcgccagcggcggcggagcgaggctgagccggtctctccggcctccggtcggcgttaagttggggatcgtaacgtgacgtgtctcgtctccacggatcgacacaaccggcctactcgggtgcacgacgccgcgataagggcgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagaatcagatcaaccggccaataggacgccagcgagcccaacacctggcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccagcactttcacgctcagcttcagcaagatctaccgtcttcggtacgcgctcactccgccctctgcctttgttactgccacgtttctctgaatgctctcttgtatggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcatacggtatttatttaagcacctgttgctgctatagggcacttgtattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgacgataagtcaagatcagatgcacttgttttaaatattgttgtctgaagaaataagtactgacagttttttgatgcattgatctgcttgtttgttgtaacaaaattttaaaataaagagttccctttttgttgctctccttacctcctgatggtatctagtatctaccaactgatactatattgcttctctttacatacgtatcttgctcgatgccttctcctagtgttgaccagtgttactcacatagtctttgctcatttcattgtaatgcagataccaagcggatgccggactccgacaacgagtccggcgggccgagcaacgcggagttctcgtcgccgcgggagcaggaccggttcctgccgatcgcgaacgtgagccggatcatgaagaaggcgctcccggccaacgccaagatctccaaggacgccaaggagacggtgcaggagtgcgtgtcggagttcatctccttcatcaccggcgaggcctccgacaagtgccagcgcgagaagcgcaagaccatcaacggcgacgacctactctgggccatgaccacgctcggcttcgaggactacgtcgagccgctcaagctctacctccacaagttccgcgagctcgagggcgagaaggcggccacgacgagcgcctcctccggcccgcagccgccgctgcacagggagacgacgccgtcgtcgtcaacgcacaatggcgcgggcgggcccgtcgggggatacggcatgtacggcggcgcgggcgggggaagcggtatgatcatgatgatgggacagcccatgtacggcggctccccgccggccgcgtcgtccgggtcgtacccgcaccaccagatggccatgggcggaaaaggtggcgcctatggctacggcggaggctcgtcgtcgtcgccgtcagggctcggcaggtaggttggttagtgttcgaggtttggtttggtgaggtgtgaagtgcctgaactctgatggttgtttgtgaggtgtggtcgcaatagctgcggcctgtggtgaacttctcagaaataaataagttttcgttcctaatttgttttttttcaattcatgtgttggtcgccttgtttcgtatcgttgcttttcatgttaaacaggtactcttgtcatgcaagtgatgatgtgaatacgaggcactgcataatttgcacaggacagacgaatgcagcagtgtttgtgattcacctcactgcctccgtgatgaaaactaatctaaaattctgtgatgaaaactaatctaaaattggatgtttgatccattgagagacaaaatatgttagtaatatttatcattgtcatgtgatgcgtgaatccagcagaactcatctggagcgaagagccgcagagcacgcagttactccctccggctaaaacagactataatttggaatgtaagagtt

SEQ ID NO: 67 - DNA, 2639 bp, Artificial Sequence
  recombinant plasmid pYTEN27
cgccgagcaaaaacacagcccgaccacaaccgacaacctgaaagaacaacagagatacacaggcatgctgggggacctagaccagcgcccagaagtaataacgccagcggagatacaaccgctccgagagagcctgaccatctgagaacacattggtcaccaaaagcaccaccaaccggcctagacaaagcagctcagttgacccccgcctcgacatcttcgatggccggcatcacctttctccccttctttttattcttcgctgtcttcaccttgtcttgatttaacagctccatgattgcatccatttgcttcttggagagaggctttgtgagaaggcttgtcatctgctcaaatgactcatcaaagttagtacattttgaagaactaattattattatatagaatgcactgcacatatattactattaccagttttcttgggcacagcagaaaacatgcacacgcagatagaaaaaggagaggccataaaccaaaaggctttaagaatatatgtaaagatatgtctaaatatatggctatatctggttaagcaagataacagggctctggtcatcagtagtagtggccttttgcccttgcccctctctctcacctctcttttctcagccttgcttccgatggatcccatcccactgccatcctttctttcccttgcgcgcattgcctagccggccggccggcctgctattaaaccactttacccgccccctctcgctcacgctcgacgcagctcccttttccttgtttgcttattgcaagtctctgcaagaacctgctagagaggaacaaggtagagtagtatcgcttttttccatctaggttatctctttttacatgaaaaatttcagccgtatttcgttctccatcagtcctgcgataatatatacgcgcgtcttgtgtgatccggcatatgtatagttcctgctaactgatcgagatcgctctcgtttgtactttctccctttgaggaaagagtttccccttttctgtgcttcaagttcttgtaaggaaaaccatgcctgccagcttcttctgctacttgtatgatgattcttatttgcttattacttgatttccgttttttttcttgctttctatatgtatgtatctgggctgtcttcccctgcgtctcgttactgctaagctttggaaggtttcaactctttgtatacgatgaggtttctgctcctagtagcagatccgcgcatatgactagatgtttgaggaaaagaaaagggcaagacgctatatatatatgcagcacgcagtcgcacatatattcagttttccaatctgcctcttgctttatgataattcaacttgcgctgattatattcttggctacctagaaatgtctaattaaactttgtttgctagctagattttgttgcttcctttcgcatctgatctttttatctcttctgagtgctccgcaaagccttccagtgttgaagaagctgctggaagaacgaagagatgccggactccgacaacgagtccggcgggccgagcaacgcggagttctcgtcgccgcgggagcaggaccggttcctgccgatcgcgaacgtgagccggatcatgaagaaggcgctcccggccaacgccaagatctccaaggacgccaaggagacggtgcaggagtgcgtgtcggagttcatctccttcatcaccggcgaggcctccgacaagtgccagcgcgagaagcgcaagaccatcaacggcgacgacctactctgggccatgaccacgctcggcttcgaggactacgtcgagccgctcaagctctacctccacaagttccgcgagctcgagggcgagaaggcggccacgacgagcgcctcctccggcccgcagccgccgctgcacagggagacgacgccgtcgtcgtcaacgcacaatggcgcgggcgggcccgtcgggggatacggcatgtacggcggcgcgggcgggggaagcggtatgatcatgatgatgggacagcccatgtacggcggctccccgccggccgcgtcgtccgggtcgtacccgcaccaccagatggccatgggcggaaaaggtggcgcctatggctacggcggaggctcgtcgtcgtcgccgtcagggctcggcaggtaggttggttagtgttcgaggtttggtttggtgaggtgtgaagtgcctgaactctgatggttgtttgtgaggtgtggtcgcaatagctgcggcctgtggtgaacttctcagaaataaataagttttcgttcctaatttgttttttttcaattcatgtgttggtcgccttgtttcgtatcgttgcttttcatgttaaacaggtactcttgtcatgcaagtgatgatgtgaatacgaggcactgcataatttgcacaggacagacgaatgcagcagtgtttgtgattcacctcactgcctccgtgatgaaaactaatctaaaattctgtgatgaaaactaatctaaaattggatgtttgatccattgagagacaaaatatgttagtaatatttatcattgtcatgtgatgcgtgaatccagcagaactcatctggagcgaagagccgcagagcacgcagttactccctccggctaaaacagactataatttggaatgtaagagtt

SEQ ID NO: 68 - DNA, 2639 bp, Artificial Sequence
  recombinant plasmid pYTEN28
gattcgcgcgtgctggaactcgggattggattcatgcgtgctggaacttggaagtctggagtggactttggaagcctggattccagaacaagaactcaagaagtctaggagccgccgagcaggtagggaattagggaaataaagagaagaggcggctggcgttcgacgttccatcttcagtagaggcggctggcgtttcaactccctgtagtcgggccgcctgccaaaaaagcccacgaaggcaggaaatcaaaaaatctaggtcctaaacctagtcgcgcagaaccggctaatcgagcgactaatcgaccctaatcgtcgactagtcggacggccagggcgattaggtactctaatcgagtcggttgtgctaatcgagctctgctaaccgactagcccgaccgcgattagtcagatgacttgaaaacaaagatagagacatacttttttatattctttgcattgttttgtttctatccaaaactgctatttagaaattggaaaatctgcacattgaaaaatctaatggattagatatgttgatttgtttttattcacgagcataatcaaataaattagatttagaattggactgcacgcagtgaactactgaactgaactgtgttcaataatttaaatactcacggctgagccgtgagctgtaggctggagcacaagcacgagccagcaccgagcggcggagcactggagcagcaggcgagcagggaggcggccaggcgggagcagccagccagcaagcaggcagcagcggagcagcccacagccgagcgcccaagctggagctgctgcagagcctgcagcgtgccgctgcgcgccggcaggacaggagcggccgagcgggagtgcaggactgtggcctgcgggacgcggggatgggcggacggcgtagcgcttacagtccgcggacagcggactcacggtggcggctaagatagtgagaccgatgacctaatctctatttggaccggttcaaggttttgcccagttaatattggaccatattgggccttccgcccctgctcgcaagacacactgaacaaagaatccacacggctctccaaaagatagagagataattcacatgcttctctctctctgaaaaaaaggaacttgcatggttgacacggaaaacgtcattaaacgcgcacgtggctgcaaatgcaacgtaacagatccatcatctatccatccatagaatcagacggccacagaaggcaacgaccgtgtgcctgtccaccggcgcaggtggcccacagacgcccgtgcgattcatccgtctcggcccaccaaccacgggaggggccccagggccctccttagtccttacaaataccggcagcagcatcacccggccaccaccacccacccgttttatccacgcacggcgtcgaacaccccgcggtcgctcacgtgaggcgccaccccgcgcacccagtcagcgcccgcctccaccacccacccacacgacaaaaatccgccatgccggactccgacaacgagtccggcgggccgagcaacgcggagttctcgtcgccgcgggagcaggaccggttcctgccgatcgcgaacgtgagccggatcatgaagaaggcgctcccggccaacgccaagatctccaaggacgccaaggagacggtgcaggagtgcgtgtcggagttcatctccttcatcaccggcgaggcctccgacaagtgccagcgcgagaagcgcaagaccatcaacggcgacgacctactctgggccatgaccacgctcggcttcgaggactacgtcgagccgctcaagctctacctccacaagttccgcgagctcgagggcgagaaggcggccacgacgagcgcctcctccggcccgcagccgccgctgcacagggagacgacgccgtcgtcgtcaacgcacaatggcgcgggcgggcccgtcgggggatacggcatgtacggcggcgcgggcgggggaagcggtatgatcatgatgatgggacagcccatgtacggcggctccccgccggccgcgtcgtccgggtcgtacccgcaccaccagatggccatgggcggaaaaggtggcgcctatggctacggcggaggctcgtcgtcgtcgccgtcagggctcggcaggtaggttggttagtgttcgaggtttggtttggtgaggtgtgaagtgcctgaactctgatggttgtttgtgaggtgtggtcgcaatagctgcggcctgtggtgaacttctcagaaataaataagttttcgttcctaatttgttttttttcaattcatgtgttggtcgccttgtttcgtatcgttgcttttcatgttaaacaggtactcttgtcatgcaagtgatgatgtgaatacgaggcactgcataatttgcacaggacagacgaatgcagcagtgtttgtgattcacctcactgcctccgtgatgaaaactaatctaaaattctgtgatgaaaactaatctaaaattggatgtttgatccattgagagacaaaatatgttagtaatatttatcattgtcatgtgatgcgtgaatccagcagaactcatctggagcgaagagccgcagagcacgcagttactccctccggctaaaacagactataatttggaatgtaagagtt

SEQ ID NO: 69 - DNA, 2646 bp, Artificial Sequence
  recombinant plasmid pYTEN29
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatgccggactccgacaacgagtccggcgggccgagcaacgcggagttctcgtcgccgcgggagcaggaccggttcctgccgatcgcgaacgtgagccggatcatgaagaaggcgctcccggccaacgccaagatctccaaggacgccaaggagacggtgcaggagtgcgtgtcggagttcatctccttcatcaccggcgaggcctccgacaagtgccagcgcgagaagcgcaagaccatcaacggcgacgacctactctgggccatgaccacgctcggcttcgaggactacgtcgagccgctcaagctctacctccacaagttccgcgagctcgagggcgagaaggcggccacgacgagcgcctcctccggcccgcagccgccgctgcacagggagacgacgccgtcgtcgtcaacgcacaatggcgcgggcgggcccgtcgggggatacggcatgtacggcggcgcgggcgggggaagcggtatgatcatgatgatgggacagcccatgtacggcggctccccgccggccgcgtcgtccgggtcgtacccgcaccaccagatggccatgggcggaaaaggtggcgcctatggctacggcggaggctcgtcgtcgtcgccgtcagggctcggcaggtaggttggttagtgttcgaggtttggtttggtgaggtgtgaagtgcctgaactctgatggttgtttgtgaggtgtggtcgcaatagctgcggcctgtggtgaacttctcagaaataaataagttttcgttcctaatttgttttttttcaattcatgtgttggtcgccttgtttcgtatcgttgcttttcatgttaaacaggtactcttgtcatgcaagtgatgatgtgaatacgaggcactgcataatttgcacaggacagacgaatgcagcagtgtttgtgattcacctcactgcctccgtgatgaaaactaatctaaaattctgtgatgaaaactaatctaaaattggatgtttgatccattgagagacaaaatatgttagtaatatttatcattgtcatgtgatgcgtgaatccagcagaactcatctggagcgaagagccgcagagcacgcagttactccctccggctaaaacagactataatttggaatgtaagagtt

SEQ ID NO: 70 - DNA, 1996 bp, Zea mays
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctataaagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaaatctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagg

SEQ ID NO: 71 - DNA, 20231 bp, Artificial Sequence
  recombinant plasmid pYTEN30
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagttatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttacctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatggtagaggagctcgccgcttggtatctgcattacaatgaaatgagcaaagactatgtgagtaacactggtcaacactagggagaaggcatcgagcaagatacgtatgtaaagagaagcaatatagtgtcagttggtagatactagataccatcaggaggtaaggagagcaacaaaaaggaaactctttatttttaaattttgttacaacaaacaagcagatcaatgcatcaaaatactgtcagtacttatttcttcagacaacaatatttaaaacaagtgcatctgatcttgacttatggtcacaataaaggagcagagataaacatcaaaatttcgtcatttatatttattccttcaggcgttaacaatttaacagcacacaaacaaaaacagaataggaatatctaattttggcaaataataagctctgcagacgaacaaattattatagtatcgcctataatatgaatccctatactattgacccatgtagtatgaagcctgtgcctaaattaacagcaaacttctgaatccaagtgccctataacaccaacatgtgcttaaataaataccgctaagcaccaaattacacatttctcgtattgctgtgtaggttctatcttcgtttcgtactaccatgtccctatattttgctgctacaaaggacggcaagtaatcagcacaggcagaacacgatttcagagtgtaattctagatccagctaaaccactctcagcaatcaccacacaagagagcattcagagaaacgtggcagtaacaaaggcagagggcggagtgagcgcgtaccgaagacggtagatctctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccccaccatggcgtgcaggtcgactctagaggatccatggcaccgaagaagaagagaaaggtcgggattcacggcgttcctgcggcgatggataagaagtatagcattgggcttgacattggcacgaactccgtgggctgggccgtcatcaccgacgagtacaaggtgccgtctaagaagttcaaggtcctgggcaacaccgatcggcactcaatcaagaagaatctcattggagcactcctgttcgactccggagagacagcggaggccacgcggctcaagaggacagcgcgcaggcggtacacgcgcaggaagaatcgcatctgctacctccaggagatcttcagcaacgagatggcgaaggtggacgattcattcttccataggctggaggagtcgttcctcgtcgaggaagacaagaagcacgagcggcatccgatcttcggcaacatcgtggacgaggtcgcctaccacgagaagtacccaacaatctaccatctgcggaagaagctcgtggactcgacggataaggcggacctccgcctgatctacctcgccctggcgcacatgatcaagttcaggggccatttcctgatcgagggcgatctcaaccctgacaattccgatgtggacaagctgttcatccagctcgtccagacctacaatcagctcttcgaggagaacccgatcaatgcctctggcgtggacgccaaggcgatcctgtcagcgaggctctctaagtcacggcgcctcgagaacctgatcgcccagctccctggcgagaagaagaacggcctgttcggcaatctcattgccctctcgctgggcctcacccctaacttcaagtccaatttcgatctcgccgaggacgcgaagctgcagctctccaaggacacatacgacgatgacctcgataacctcctggcccagatcggcgatcagtacgcggacctgttcctcgccgcgaagaatctgtctgacgccatcctcctgtcagatatcctcagggtgaacaccgagatcacaaaggccccgctctcggcgtccatgatcaagcgctacgacgagcaccatcaggatctgaccctcctgaaggcgctggtcaggcagcagctcccagagaagtacaaggagatcttcttcgatcagtccaagaatggctacgcgggatacattgatggcggcgctagccaagaggagttctacaagttcatcaagccgatcctggagaagatggatggcacggaggagctcctggtgaagctcaatcgcgaggacctcctgaggaagcagcggaccttcgataacggcagcatcccacaccagatccatctcggcgagctgcatgcaatcctgcgccgccaagaggacttctaccctttcctcaaggataaccgcgagaagatcgagaagatcctgacgttcaggattccttactacgtgggaccactggccaggggcaatagccgcttcgcgtggatgacccgcaagtctgaggagacaatcacgccgtggaacttcgaggaagtggtcgataagggcgccagcgcgcagtctttcatcgagaggatgaccaatttcgacaagaacctgcctaatgagaaggtgctcccgaagcattccctcctgtacgagtacttcaccgtctacaacgagctcacaaaggtgaagtatgtgacggagggcatgaggaagccagccttcctgagcggcgagcagaagaaggcgatcgtggacctcctgttcaagaccaatcggaaggtgacagtcaagcagctcaaggaagactacttcaagaagatcgagtgcttcgattctgtggagatctcaggcgtcgaggaccgcttcaacgcctccctcggcacataccacgatctcctgaagatcatcaaggataaggacttcctggacaacgaggagaatgaggatatcctcgaggacatcgtgctgaccctcacactgttcgaggatcgggagatgatcgaggagcgcctgaagacatacgcccatctcttcgatgacaaggtcatgaagcagctcaagcgcaggcggtacaccggatggggccgcctgtcaaggaagctcatcaatggcatccgggacaagcagtcaggcaagacaatcctcgacttcctgaagtcggatggcttcgcgaaccgcaatttcatgcagctgatccacgatgactctctcaccttcaaggaagacattcagaaggcccaggtgagcggacagggcgactctctgcacgagcatatcgccaacctcgcgggctcaccagcgatcaagaagggcatcctgcagaccgtgaaggtggtcgatgagctcgtgaaggtcatgggcaggcataagcctgagaacatcgtcatcgagatggcccgcgagaatcagaccacacagaagggccagaagaactctcgcgagaggatgaagaggatcgaggaaggcatcaaggagctgggctcacagatcctcaaggagcacccggtggagaacacacagctgcagaatgagaagctctacctgtactacctccagaatggccgcgatatgtatgtggaccaggagctggatatcaacaggctctcggattacgacgtggatcatatcgtccctcagtcgttcctgaaggatgactccatcgacaataaggtgctcacgaggtccgacaagaaccggggcaagtcagataatgtcccgtcggaggaagtggtcaagaagatgaagaactactggcgccagctcctgaatgccaagctgatcacccagcggaagttcgataacctcacaaaggcggagcgcggcggcctctctgagctggacaaggcgggcttcatcaagaggcagctggtggagacacgccagatcacaaagcacgtcgcgcagatcctcgattctcggatgaacaccaagtacgatgagaatgacaagctgatccgcgaggtgaaggtcatcacactgaagtcgaagctcgtgtccgacttcaggaaggatttccagttctacaaggtccgggagatcaacaattaccaccatgcccatgacgcctacctcaatgcggtggtgggcacggcgctgatcaagaagtacccaaagctcgagtccgagttcgtgtacggcgactacaaggtgtacgatgtcaggaagatgatcgccaagtccgagcaggagatcggcaaggccaccgcgaagtacttcttctacagcaacatcatgaatttcttcaagacggagatcaccctggccaatggcgagatccggaagcgccctctcatcgagacgaacggcgagacaggcgagatcgtgtgggacaagggccgcgatttcgcgaccgtgaggaaggtcctctcaatgccgcaggtcaatatcgtcaagaagacagaggtccagacgggcggcttctcaaaggagtcgatcctgccaaagcggaactcggataagctcatcgcccgcaagaaggactgggatccaaagaagtacggcggattcgacagccctaccgtggcctactctgtcctggtggtcgcgaaggtggagaagggcaagtccaagaagctcaagagcgtcaaggagctcctgggcatcacaatcatggagcgctccagcttcgagaagaatccaatcgatttcctcgaggcgaagggctacaaggaagtgaagaaggacctgatcatcaagctccctaagtactctctcttcgagctggagaacggcaggaagcggatgctcgcctcagcgggcgagctccagaagggcaatgagctcgccctgccgtccaagtatgtgaacttcctctacctggcgtcccactacgagaagctcaagggcagcccagaggataacgagcagaagcagctgttcgtcgagcagcacaagcattacctcgacgagatcatcgagcagatctccgagttctccaagcgcgtgatcctcgccgacgcgaatctggataaggtcctcagcgcctacaacaagcaccgcgacaagcctatcagggagcaggcggagaatatcatccatctcttcacgctgaccaacctcggcgccccggccgcgttcaagtacttcgacacgaccatcgatcgcaagaggtacacatctacgaaggaagtgctggatgcgaccctcatccaccagtccatcacgggcctgtacgagacacgcatcgacctcagccagctcggaggtgacaagagacccgcagcaacaaagaaggcagggcaggctaagaagaagaagtgacaattcgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccgttaatcgccccgggggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtgctccgctctctcaaactcccgttttagagctagaaatagcaagttaaaataaggctagtccgttatcaacttgaaaaagtggcaccgagtcggtgctttttttgtcccttcgaagggcaattctgcagatatccatcacactggcggccgctcgaggtcgacggtatcgatcctaggttaatcgccccgggggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtgcaacggcgacgaaacgagtggttttagagctagaaatagcaagttaaaataaggctagtccgttatcaacttgaaaaagtggcaccgagtcggtgctttttttgtcccttcgaagggcaattctgcagatatccatcacactggcggccgctcgaggtcgacggtatcgatcctaggttaatcgccccggggtcccttcgaagggcaattctgcagatatccatcacactggcggccgctcgaggtcgacggtatcgatcctaggttaattaacctcactcgtttcgtcgccgttgtcggcagatgtgcataatttatatgtagctatggtcatttatgctatgctttcatattgatactatagctttgttctaagaaaataaatatgtatatatcatataacgatatatatttttatcaatgttgagcattgtataccaaattattatactagatatttctaatattagtagattgttagggtttctattttatagtatgtctttttttagttgttggcttatagctagtaacctttttagatattggctagctaatctataccactatataatctatcagttaaaattttacaaaactcatccaactaaatcactttacccatagccttgctaaatcaaccaaacaaattgcacctagacttaacacacaatcacaaccaacggttcatatcgctagataaacctctctcccttacacacttaggtgttgtttggttccatgcgaggagcggtaatgtcaatgggagccgttgtcgatgctatgcattgcatattaacaactaacacgctttgtttggtggtcggttgggaacaaaggttacaatgagagatgggacgggatcgaataacttcgggtgggtcgcggtaacaagattgtactactctgtatctgatagcgccgcgttccgctgcaggctcggtcaaccaaacgatatgtaaccggaccgggtcacaccgtaaatcgatcgcgggggtaaataacgcaaacccaacatcaccttaaatccaatgaataatattatttagatcattcatcctcctcctccacccacgaccacgacgtcccctcccctcacctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctataaagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaaatctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggccccggctccccgctctcccccgcccgtccagctcgtgctccgcctccgctgctctgccctcttcctcctctgcgtttctcctcagagctgtttgacttgaccggacagtgctgttcggtggctcggccgcgatgccggactccgacaacgagtccggcgggccgagcaacgcggagttctcgtcgccgcgggagcaggaccggttcctgccgatcgcgaacgtgagccggatcatgaagaaggcgctcccggccaacgccaagatctccaaggacgccaaggagacggtgcaggagtgcgtgtcggagttcatctccttcatcaccggcgaggcctccgacaagtgccagcgcgagaagcgcaagaccatcaacggcgacgacctactctgggccatgaccacgctcggcttcgaggactacgtcgagccgctcaagctctacctccacaagttccgcgagctcgagggcgagaaggcggccacgacgagcgcctcctccggcccgcagccgccgctgcacagggagacgacgccgtcgtcgtcaacgcacaatggcgcgggcgggcccgtcgggggatacggcatgtacggcggcgcgggcgggggaagcggtatgatcatgatgatgggacagcccatgtacggcggctccccgccggccgcgtcgtccgggtcgtacccgcaccaccagatggccatgggcggaaaaggtggcgcctatggctacggcggaggctcgtcgtcgtcgccgtcagggctcggcaggtaggacaggttgtgaccgtcgccgtccatgcttgcctccgctctctcaaactccccggggcgcgccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 72 - DNA, 16437 bp, Artificial Sequence
  recombinant plasmid pYTEN31
ttagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggaagcttggcgcgcccctaggctgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagttatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttacctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatggtagaggagctcgccgcttggtatctgcattacaatgaaatgagcaaagactatgtgagtaacactggtcaacactagggagaaggcatcgagcaagatacgtatgtaaagagaagcaatatagtgtcagttggtagatactagataccatcaggaggtaaggagagcaacaaaaaggaaactctttatttttaaattttgttacaacaaacaagcagatcaatgcatcaaaatactgtcagtacttatttcttcagacaacaatatttaaaacaagtgcatctgatcttgacttatggtcacaataaaggagcagagataaacatcaaaatttcgtcatttatatttattccttcaggcgttaacaatttaacagcacacaaacaaaaacagaataggaatatctaattttggcaaataataagctctgcagacgaacaaattattatagtatcgcctataatatgaatccctatactattgacccatgtagtatgaagcctgtgcctaaattaacagcaaacttctgaatccaagtgccctataacaccaacatgtgcttaaataaataccgctaagcaccaaattacacatttctcgtattgctgtgtaggttctatcttcgtttcgtactaccatgtccctatattttgctgctacaaaggacggcaagtaatcagcacaggcagaacacgatttcagagtgtaattctagatccagctaaaccactctcagcaatcaccacacaagagagcattcagagaaacgtggcagtaacaaaggcagagggcggagtgagcgcgtaccgaagacggtagatctctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccccaccatggcgtgcaggtcgactctagaggatccatggcaccgaagaagaagagaaaggtcgggattcacggcgttcctgcggcgatggataagaagtatagcattgggcttgacattggcacgaactccgtgggctgggccgtcatcaccgacgagtacaaggtgccgtctaagaagttcaaggtcctgggcaacaccgatcggcactcaatcaagaagaatctcattggagcactcctgttcgactccggagagacagcggaggccacgcggctcaagaggacagcgcgcaggcggtacacgcgcaggaagaatcgcatctgctacctccaggagatcttcagcaacgagatggcgaaggtggacgattcattcttccataggctggaggagtcgttcctcgtcgaggaagacaagaagcacgagcggcatccgatcttcggcaacatcgtggacgaggtcgcctaccacgagaagtacccaacaatctaccatctgcggaagaagctcgtggactcgacggataaggcggacctccgcctgatctacctcgccctggcgcacatgatcaagttcaggggccatttcctgatcgagggcgatctcaaccctgacaattccgatgtggacaagctgttcatccagctcgtccagacctacaatcagctcttcgaggagaacccgatcaatgcctctggcgtggacgccaaggcgatcctgtcagcgaggctctctaagtcacggcgcctcgagaacctgatcgcccagctccctggcgagaagaagaacggcctgttcggcaatctcattgccctctcgctgggcctcacccctaacttcaagtccaatttcgatctcgccgaggacgcgaagctgcagctctccaaggacacatacgacgatgacctcgataacctcctggcccagatcggcgatcagtacgcggacctgttcctcgccgcgaagaatctgtctgacgccatcctcctgtcagatatcctcagggtgaacaccgagatcacaaaggccccgctctcggcgtccatgatcaagcgctacgacgagcaccatcaggatctgaccctcctgaaggcgctggtcaggcagcagctcccagagaagtacaaggagatcttcttcgatcagtccaagaatggctacgcgggatacattgatggcggcgctagccaagaggagttctacaagttcatcaagccgatcctggagaagatggatggcacggaggagctcctggtgaagctcaatcgcgaggacctcctgaggaagcagcggaccttcgataacggcagcatcccacaccagatccatctcggcgagctgcatgcaatcctgcgccgccaagaggacttctaccctttcctcaaggataaccgcgagaagatcgagaagatcctgacgttcaggattccttactacgtgggaccactggccaggggcaatagccgcttcgcgtggatgacccgcaagtctgaggagacaatcacgccgtggaacttcgaggaagtggtcgataagggcgccagcgcgcagtctttcatcgagaggatgaccaatttcgacaagaacctgcctaatgagaaggtgctcccgaagcattccctcctgtacgagtacttcaccgtctacaacgagctcacaaaggtgaagtatgtgacggagggcatgaggaagccagccttcctgagcggcgagcagaagaaggcgatcgtggacctcctgttcaagaccaatcggaaggtgacagtcaagcagctcaaggaagactacttcaagaagatcgagtgcttcgattctgtggagatctcaggcgtcgaggaccgcttcaacgcctccctcggcacataccacgatctcctgaagatcatcaaggataaggacttcctggacaacgaggagaatgaggatatcctcgaggacatcgtgctgaccctcacactgttcgaggatcgggagatgatcgaggagcgcctgaagacatacgcccatctcttcgatgacaaggtcatgaagcagctcaagcgcaggcggtacaccggatggggccgcctgtcaaggaagctcatcaatggcatccgggacaagcagtcaggcaagacaatcctcgacttcctgaagtcggatggcttcgcgaaccgcaatttcatgcagctgatccacgatgactctctcaccttcaaggaagacattcagaaggcccaggtgagcggacagggcgactctctgcacgagcatatcgccaacctcgcgggctcaccagcgatcaagaagggcatcctgcagaccgtgaaggtggtcgatgagctcgtgaaggtcatgggcaggcataagcctgagaacatcgtcatcgagatggcccgcgagaatcagaccacacagaagggccagaagaactctcgcgagaggatgaagaggatcgaggaaggcatcaaggagctgggctcacagatcctcaaggagcacccggtggagaacacacagctgcagaatgagaagctctacctgtactacctccagaatggccgcgatatgtatgtggaccaggagctggatatcaacaggctctcggattacgacgtggatcatatcgtccctcagtcgttcctgaaggatgactccatcgacaataaggtgctcacgaggtccgacaagaaccggggcaagtcagataatgtcccgtcggaggaagtggtcaagaagatgaagaactactggcgccagctcctgaatgccaagctgatcacccagcggaagttcgataacctcacaaaggcggagcgcggcggcctctctgagctggacaaggcgggcttcatcaagaggcagctggtggagacacgccagatcacaaagcacgtcgcgcagatcctcgattctcggatgaacaccaagtacgatgagaatgacaagctgatccgcgaggtgaaggtcatcacactgaagtcgaagctcgtgtccgacttcaggaaggatttccagttctacaaggtccgggagatcaacaattaccaccatgcccatgacgcctacctcaatgcggtggtgggcacggcgctgatcaagaagtacccaaagctcgagtccgagttcgtgtacggcgactacaaggtgtacgatgtcaggaagatgatcgccaagtccgagcaggagatcggcaaggccaccgcgaagtacttcttctacagcaacatcatgaatttcttcaagacggagatcaccctggccaatggcgagatccggaagcgccctctcatcgagacgaacggcgagacaggcgagatcgtgtgggacaagggccgcgatttcgcgaccgtgaggaaggtcctctcaatgccgcaggtcaatatcgtcaagaagacagaggtccagacgggcggcttctcaaaggagtcgatcctgccaaagcggaactcggataagctcatcgcccgcaagaaggactgggatccaaagaagtacggcggattcgacagccctaccgtggcctactctgtcctggtggtcgcgaaggtggagaagggcaagtccaagaagctcaagagcgtcaaggagctcctgggcatcacaatcatggagcgctccagcttcgagaagaatccaatcgatttcctcgaggcgaagggctacaaggaagtgaagaaggacctgatcatcaagctccctaagtactctctcttcgagctggagaacggcaggaagcggatgctcgcctcagcgggcgagctccagaagggcaatgagctcgccctgccgtccaagtatgtgaacttcctctacctggcgtcccactacgagaagctcaagggcagcccagaggataacgagcagaagcagctgttcgtcgagcagcacaagcattacctcgacgagatcatcgagcagatctccgagttctccaagcgcgtgatcctcgccgacgcgaatctggataaggtcctcagcgcctacaacaagcaccgcgacaagcctatcagggagcaggcggagaatatcatccatctcttcacgctgaccaacctcggcgccccggccgcgttcaagtacttcgacacgaccatcgatcgcaagaggtacacatctacgaaggaagtgctggatgcgaccctcatccaccagtccatcacgggcctgtacgagacacgcatcgacctcagccagctcggaggtgacaagagacccgcagcaacaaagaaggcagggcaggctaagaagaagaagtgacaattcgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccgttaatcgccccgggggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtgctccgctctctcaaactcccgttttagagctagaaatagcaagttaaaataaggctagtccgttatcaacttgaaaaagtggcaccgagtcggtgctttttttgtcccttcgaagggcaattctgcagatatccatcacactggcggccgctcgaggtcgacggtatcgatcctaggttaatcgccccgggggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtgcaacggcgacgaaacgagtggttttagagctagaaatagcaagttaaaataaggctagtccgttatcaacttgaaaaagtggcaccgagtcggtgctttttttgtcccttcgaagggcaattctgcagatatccatcacactggcggccgctcgaggtcgacggtatcgatcctaggttaatcgccccggggtcccttcgaagggcaattctgcagatatccatcacactggcggccgctcgaggtcgacggtatcgatcctaggttaattaacctcactcgtttcgtcgccgttgtcggcagatgtgcataatttatatgtagctatggtcatttatgctatgctttcatattgatactatagctttgttctaagaaaataaatatgtatatatcatataacgatatatatttttatcaatgttgagcattgtataccaaattattatactagatatttctaatattagtagattgttagggtttctattttatagtatgtctttttttagttgttggcttatagctagtaacctttttagatattggctagctaatctataccactatataatctatcagttaaaattttacaaaactcatccaactaaatcactttacccatagccttgctaaatcaaccaaacaaattgcacctagacttaacacacaatcacaaccaacggttcatatcgctagataaacctctctcccttacacacttaggtgttgtttggttccatgcgaggagcggtaatgtcaatgggagccgttgtcgatgctatgcattgcatattaacaactaacacgctttgtttggtggtcggttgggaacaaaggttacaatgagagatgggacgggatcgaataacttcgggtgggtcgcggtaacaagattgtactactctgtatctgatagcgccgcgttccgctgcaggctcggtcaaccaaacgatatgtaaccggaccgggtcacaccgtaaatcgatcgcgggggtaaataacgcaaacccaacatcaccttaaatccaatgaataatattatttagatcattcatcctcctcctccacccacgaccacgacgtcccctcccctcacctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctataaagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaaatctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggccccggctccccgctctcccccgcccgtccagctcgtgctccgcctccgctgctctgccctcttcctcctctgcgtttctcctcagagctgtttgacttgaccggacagtgctgttcggtggctcggccgcgatgccggactccgacaacgagtccggcgggccgagcaacgcggagttctcgtcgccgcgggagcaggaccggttcctgccgatcgcgaacgtgagccggatcatgaagaaggcgctcccggccaacgccaagatctccaaggacgccaaggagacggtgcaggagtgcgtgtcggagttcatctccttcatcaccggcgaggcctccgacaagtgccagcgcgagaagcgcaagaccatcaacggcgacgacctactctgggccatgaccacgctcggcttcgaggactacgtcgagccgctcaagctctacctccacaagttccgcgagctcgagggcgagaaggcggccacgacgagcgcctcctccggcccgcagccgccgctgcacagggagacgacgccgtcgtcgtcaacgcacaatggcgcgggcgggcccgtcgggggatacggcatgtacggcggcgcgggcgggggaagcggtatgatcatgatgatgggacagcccatgtacggcggctccccgccggccgcgtcgtccgggtcgtacccgcaccaccagatggccatgggcggaaaaggtggcgcctatggctacggcggaggctcgtcgtcgtcgccgtcagggctcggcaggtaggacaggttgtgaccgtcgccgtccatgcttgcctccgctctctcaaactccccggggcgcgccaagcttgtttaaacgaattcaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgc

SEQ ID NO: 73 - DNA, 3664 bp, Artificial Sequence
  recombinant plasmid pYTEN32
ttaattaacctcactcgtttcgtcgccgttgtcggcagatgtgcataatttatatgtagctatggtcatttatgctatgctttcatattgatactatagctttgttctaagaaaataaatatgtatatatcatataacgatatatatttttatcaatgttgagcattgtataccaaattattatactagatatttctaatattagtagattgttagggtttctattttatagtatgtctttttttagttgttggcttatagctagtaacctttttagatattggctagctaatctataccactatataatctatcagttaaaattttacaaaactcatccaactaaatcactttacccatagccttgctaaatcaaccaaacaaattgcacctagacttaacacacaatcacaaccaacggttcatatcgctagataaacctctctcccttacacacttaggtgttgtttggttccatgcgaggagcggtaatgtcaatgggagccgttgtcgatgctatgcattgcatattaacaactaacacgctttgtttggtggtcggttgggaacaaaggttacaatgagagatgggacgggatcgaataacttcgggtgggtcgcggtaacaagattgtactactctgtatctgatagcgccgcgttccgctgcaggctcggtcaaccaaacgatatgtaaccggaccgggtcacaccgtaaatcgatcgcgggggtaaataacgcaaacccaacatcaccttaaatccaatgaataatattatttagatcattcatcctcctcctccacccacgaccacgacgtcccctcccctcacctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctataaagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaaatctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggccccggctccccgctctcccccgcccgtccagctcgtgctccgcctccgctgctctgccctcttcctcctctgcgtttctcctcagagctgtttgacttgaccggacagtgctgttcggtggctcggccgcgatgccggactccgacaacgagtccggcgggccgagcaacgcggagttctcgtcgccgcgggagcaggaccggttcctgccgatcgcgaacgtgagccggatcatgaagaaggcgctcccggccaacgccaagatctccaaggacgccaaggagacggtgcaggagtgcgtgtcggagttcatctccttcatcaccggcgaggcctccgacaagtgccagcgcgagaagcgcaagaccatcaacggcgacgacctactctgggccatgaccacgctcggcttcgaggactacgtcgagccgctcaagctctacctccacaagttccgcgagctcgagggcgagaaggcggccacgacgagcgcctcctccggcccgcagccgccgctgcacagggagacgacgccgtcgtcgtcaacgcacaatggcgcgggcgggcccgtcgggggatacggcatgtacggcggcgcgggcgggggaagcggtatgatcatgatgatgggacagcccatgtacggcggctccccgccggccgcgtcgtccgggtcgtacccgcaccaccagatggccatgggcggaaaaggtggcgcctatggctacggcggaggctcgtcgtcgtcgccgtcagggctcggcaggtaggacaggttgtgaccgtcgccgtccatgcttgcctccgctctctcaaactccccggggcgcgcc

SEQ ID NO: 74 - DNA, 1500 bp, Zea mays
gattcgcgcgtgctggaactcgggattggattcatgcgtgctggaacttggaagtctggagtggactttggaagcctggattccagaacaagaactcaagaagtctaggagccgccgagcaggtagggaattagggaaataaagagaagaggcggctggcgttcgacgttccatcttcagtagaggcggctggcgtttcaactccctgtagtcgggccgcctgccaaaaaagcccacgaaggcaggaaatcaaaaaatctaggtcctaaacctagtcgcgcagaaccggctaatcgagcgactaatcgaccctaatcgtcgactagtcggacggccagggcgattaggtactctaatcgagtcggttgtgctaatcgagctctgctaaccgactagcccgaccgcgattagtcagatgacttgaaaacaaagatagagacatacttttttatattctttgcattgttttgtttctatccaaaactgctatttagaaattggaaaatctgcacattgaaaaatctaatggattagatatgttgatttgtttttattcacgagcataatcaaataaattagatttagaattggactgcacgcagtgaactactgaactgaactgtgttcaataatttaaatactcacggctgagccgtgagctgtaggctggagcacaagcacgagccagcaccgagcggcggagcactggagcagcaggcgagcagggaggcggccaggcgggagcagccagccagcaagcaggcagcagcggagcagcccacagccgagcgcccaagctggagctgctgcagagcctgcagcgtgccgctgcgcgccggcaggacaggagcggccgagcgggagtgcaggactgtggcctgcgggacgcggggatgggcggacggcgtagcgcttacagtccgcggacagcggactcacggtggcggctaagatagtgagaccgatgacctaatctctatttggaccggttcaaggttttgcccagttaatattggaccatattgggccttccgcccctgctcgcaagacacactgaacaaagaatccacacggctctccaaaagatagagagataattcacatgcttctctctctctgaaaaaaaggaacttgcatggttgacacggaaaacgtcattaaacgcgcacgtggctgcaaatgcaacgtaacagatccatcatctatccatccatagaatcagacggccacagaaggcaacgaccgtgtgcctgtccaccggcgcaggtggcccacagacgcccgtgcgattcatccgtctcggcccaccaaccacgggaggggccccagggccctccttagtccttacaaataccggcagcagcatcacccggccaccaccacccacccgttttatccacgcacggcgtcgaacaccccgcggtcgctcacgtgaggcgccaccccgcgcacccagtcagcgcccgcctccaccacccacccacacgacaaaaatccgcc

SEQ ID NO: 75 - DNA, 1378 bp, Zea mays
atgtgttttggcacttatgtgctcgaagattatgccaaggcaaccgtttgtttcatgatgccttaggcatgcgaaattggcatagttttttatggcaatgaccgacgtccattttctcgattaaatgttttctattggctttgttcacaaaaaatgaggtaccatcgaaatcacgtttgctagtcatgtgataaatattaattagagaaaatggtgatgtaccccttcacagaagacacaattttggcacatatttaatagttctatttgcataggtgcatcatatggaacacctatttttcattgtatggggttataattatgctataatatatattaataaatatggtggatgtcgttttctaaacattccaatgcttcaaatttgacattgaagatatggatgtcaaagcattaatggctaatcacctaaactgtgagtagatttaaggtgtgcctttgtaaaactccaatctagtgaggggcaatattaaaatgtcctagtcctttctatcttccggctatcgcaccgttgttactatagcttttccgtttctaacccagtggcttccgcttgacgctctaccgaatgatttgggccccatgtgtttgggcacttatgtgcccgcaaattatgccaagacaaccatttattttatgaccttaccttagacatgtgaaagtgacgtagttttatcgacaacactcgacgtccgtttccacaattaaatgtctgttgctggttttgtttgcaaaacatgaggcgtatgtttgctagtcatgtgactaattagagagaatgatgatgtagcccttgatatgaaggcacaactttggcacatatttattagttatattcgcatcaacacaatatatggaacacctatttttcgtcgcatggggtatagttatgctacattatgttaataaatattttagaggttgtttttttataaaatctcaatactctaaagttgacatcggagcattggatatcaaagtattaatgtctaatcatctagattgagagtagattttgagatgtacctctgcagacctctacatccacgaaggagcaatttcgcaaatatactagtcctagctatcttctgcctactttgtaccattattgctgtagctttccgtttcgcctccgtcgactgacgtgtggaccccatcgcacctccctccctctccctctccccccccccacccccacgcgcctggccgcctgcagactgtgccagtggcggccagccaatatatagggcccacgccccgtccgcgctctccaaagtttcgtccaccatttcgctctcgcaagtcgcaagccaccgatttgtttacaggcgccggagctcccgacgacgactgagcc

SEQ ID NO: 76 - DNA, 1387 bp, Zea mays
gccgctccgctctcctctatggtaacttaacatcaaaaaataagaatggagccattccatttccctcagctcctcaaccaaacacaccataaccacgtcctcgtaggcagaaccgcagtagtcaccgttggccctgcatcggacggcctctgccacgctccatcgcccacccaatcatccacgcgctatagatgtccaaatggacgggtcgtgtcggcccggcccgagcacggctaggtccggcacggattaggaccctgccggctcggccccgattggctagcggcccgtgcggtgccggcccacgggccccaagcgagacccaggcatggcctgctaggctaataaccgtgtcgggtcggcccacagcccgacgggtctaacgggcccgtaacaacatatgccgtttaaacgataaaaatatcttaaaattatatatttttaagagtttaaaccatatttattagctctaaacatttatttacacccacataactaacaaaaacacttgtttcttatattttatactctatattcaagtataatatatgtatttatatgaaaaaatagggaaaaaataaaattgggccgtctcgggtctagcctaccgtgccacgttttcgattcagatacggtccggtgtctggaccgaaccggtacgagtccagttcactatgtgttcagtgccaaattcgcggcaggtcaaatccatgccggatttcggatcgcccggtccgtttagacatctaccacgggcacttgtggccgcgcgcgcatctcctaaggctatccgcaaccgttacccctaaattttttcccctatatcacttttttcccctattttcccccctattttttcatctcccgcagcggttccccctaaatactcccctataccccactacaactataaaatatcattttttatatcaactatcaattttttatctactaacaattactcgtggacccacaacacagtgtttagggtgatgaacagtgatacgctagatctgggggggagagagaagggtgccgacacgtagggggcaccgctgcggccgtagggtgccccctaccgccggcatgcaaggggagggggattgcaagggggcagcgttgcgcacagcctaagtcactcactcactcctcacgcgtctactctactacttaacacgcgcccgtgccatccgcccactcctctcccacgcactccgcaattccgcatcacgcagaggcagagcgaccaacccagaaccccaccccaccgcccgcaaccgcaagctcagattccctccccaccccaccccaccccaccgtcccgctcactccagcccagcccgcgtccccacagcccagcgacagcgggcaccggcggcatccagcc

SEQ ID NO: 77 - DNA, 1565 bp, Zea mays
gcagcagagaaaaaaaatcgccgccccctcccctggatagcccgacggctgctagcaacagaggagtagaagaaatggggagggcggagaccttgccggcaaacagcagcacgtcgtgggaagcttcgtcggccctcgttctcagttctcacgcattccagagggcagggcgttggggaaagtgaacagacgctccgtcgcattccagagggcaatggttggggaaagtgaatagcgcgcggaaagtgaaaagacgcctgagtaaaaaatcaaacagtgcaatagggaccctctgtcagcgacagtagacaaaaaaatatccaacagatgaaaaaaaagcatatggcagatccaacggtgcacaaacgtggatgcgtagtccagtgcttcttccggttgcttgttctcagcctgaaatgcctgcgccgggttggggtaacaagcctctttcgctgtgtcgtcgccgtggttgaagagctgagacgcgacgaacttgtcgagcactaaaggcaggaggaggaggcttggggctctccaccaaccgagggcgcgcgagaagcctttgctcagggaaacaaacagacagacgcaactcaggatcgataactgacactcgattccttgaagcgatgaagtggaggcgagcatgaacctcgcggggaggtagcggcggaggcatctcgtcccgaccacggcggggaccatgaaagggcggctctgggagctagagctagccgtgcgcgccggcgtgatggagagagggaagcgcggtccagtagagcgagtagtgagcggtggtagagctgctggcttgccggtagagctgctggcttagccagtggtagagctgctggcttgccgtggtagaggcgatggacccggcagggcgttgggaagatgaacgggcggtcagtcgcattccagagagtaaagatgaaagtgaacagcggaaagcgaaaagacgcaatagggaccctctgtcaagcgacagtaaacagagaaaaatatccaatagatgaaaaagagcatcctggagatccaacggcgtacaaatcgagtgaaaacaacaagagaaacacacgcgtgtccgttagctttttcctttttgttttatcagtttcggttcctatagtaaaaagttggagtcgtgctaaacggaccttacaaactccgtcaaggcaacatctaaaattcaatttatatattttttcctttgtctgttttcataaaatgcaaaactaagactttctctagcagattgtacatataacatgtaaaacatcattttacaccgtagagaacgtcacaacactgtttatagaagatggcgagaaagcctaagaaagggaacccaatgcaacgtggctcaccacgtggcacatcacaattcatcgtgtcatccagtaagctgcgctaggcgtgtggccatgtggcatatcttgctagtcaccatgccaagtcatccgccaacggtcctactgtcctagctagtttataaatctcctctctccgccggcaaccttattggtatcaagaaaaacttgtcgaaaaatacacagggcaca

SEQ ID NO: 78 - DNA, 1200 bp, Zea mays
caggagcacatgcttttgtcataaacacagactctgtcggtcaccccgaggctgtccaatagaagggacaattctggcccaacatacctcacatgtgtcgcttgtaaggtgtgttgggccgaaacgggtgacgcctaggtttcgtttgttcacactacgatacactatgattaactagaatcatcgtgcattattaaataataaaactaataaaccacaatacaatattacatacgaaataacattgttatggatgacaaatcatataaaatgaccttatttccgagggcataataattaccctcggaaattaaaaatttaaattatctaaagcaccactaaatgaactaaaacaagctaatttaattacataatcaaattttggccgtcggacataacaaactaaacaatcaaatacattcatacatacaattctaatcaaatacattcatacatacaattcttatcaaatacattaatacatacaattctaaaaaacaatgttaaggatactcacttagcggtagttgtggttatgccgttggcgaggagcagcagcgcgggagacaccggacagcgggcgtggggagccggacggcggcgggcgcacggtgcgaccggggcctcggcgagcgcgcaggcacgacggctgcctcggtgagcgcgctggcgcggcggcgacgaagcgggcacggcggcgaggcgacggcgcggagagagagagagagcaccgagcgcgcgagagagaaatgaaaatcgaaacggccctagcgccgttaccttgaaatcagtaatctccgacggctacgctgccagccgtcggacataaccttatctccgacggcctcgtacacagccgtcggagataagcagtatttccgagagcccgttgtggccgtcggacataaccttatctccgacggcctcgtaggcggccgtcagagataaggtggccgtcggatgtttgttagtttactgtagtggaaggcgccgctatcttgtcttttccgaattccgacatgcatgctgctaataaagcaatattagtattattattagagatgtcccaagttttaagataaccttttttaccctttcggaactaagtttacagtacaaaaggtttcatcttcctttctctctcttactagccaacacagtaaataaacaataaataataaagataaaagagaaggcatt

SEQ ID NO: 79 - DNA, 1200 bp, Zea mays
gctagtgtgttggccactactgaaggtcaccaggtgggatgtcctcttcctaaagaaagtgtttgctataaacatgtcataggctagggcaaagttcaggatttcctctccttcttgattcctagtcccaaagccgaagcctccatgcacaccctcgaaactgatgctagatgtacccacatggccattgagatctcctcctatgaagagcttctcgccaactggcacactactaatcatgtcctccaggccttcccagaactccctctttgagttctcattaaggcctacttgaggagcatacgcgctgataacattgagaaccaagtccccaatgaccagcttgactatgatgattctatctcctaccctcttaacatctactactccatctttaaggctcttatcgatcaagacgcctactccattcttgtttgttgcagtccccgtgtaccacaacttgaagcctgtaccttccacttccttcgccttctgtcctttccacttggtctcttgaacgcataaattatttactcgtctcctcaccgcaacgtcaactatttctcgtaacttacctgttaaggaacctacgttcccaactacctacacggaccctagttggctcgactaacttccttacccttcgcacccgccgaggaagatgcaaagacccttgctcattttccactacacccgggcgtcgatgtagcgcgccactaaggatgcgacgacccgatccttgctcatttatcaccggctccagatcaagagacggcgcgtcacttaaggggtgacggcccggcccttgctcatttaacaccatacccgggttcccgatatggcgcgtcgctaagagggttacgccccaacgattttctttgtggtttcatctccataagatttggctggtttttacgttggtttgccggacctaacacaaccctcctcctttaccgggcttgggaccggctatgttgaaacgacttaaggaagtcttccaatcaacataggcggagttagaaaaaatatactttctcgttctaaaatagtaatcgttttagctcctagaattatgtatatatttaaataaatataatgatatatatatatatatatatatatatataacacatatgaattcaccatttctctaaaataaattttatttttgtatggagagagtataaaagatcgtgtaaa

SEQ ID NO: 80 - DNA, 1200 bp, Zea mays
caaattgaagttaggacttgggaggaactcttgaaattgctcttgacaaatctaaacaaaggcctcggtcctctacctcttcacctctcccccacaaagagagaaaaaaatgatcatgttgttgttattgtctcaaccaacagaaggtataggaatccaaaaataatagtactcccttagttccacaatagtgtttgttttggctccttattttttggtctatatctatatggataacaataaatctagatacatatataaaatatatacaccaaatatagtacgaatccagacactttaaaacaaatactctctttgtttcaaaataaaagtcgttttaggcttttaacaagttcatttgataattgatgtatatgttcacattcattgtcatttatttgaatatagacataaaaatgaaaaaactaaaataactactgacagagagggagtattatttagaaacgggcggagtaacaaaattctgacactcaaacaaaacataacaaaacttcattttatttaacgaaatgtttatgactcgacgacggcaccaaatcgaagatttggtcgtattactttatacatactttggacctgtgagcaaaaagaattattggcacaacgtgtcagccgttctgaaataaaatgtctttgtgataataacaagcacaagtaaagatatagccgctttaatcggtgacagtaactagcctaggcgcgtgctgtcgggaggcagttggttataaagggatggagcctgatcgagtcagtcccgtccttatcgcctctctctgttcgcacgcgactaacagccagcccccgacacgtgaacaagcacaagggcgtcgcctcgcctcgcgcgcgctggcgccgcgcacagacacgcaagcgtggatcgcgccgccgctcatcggggcatcgtccgtcgcgacaggggtccgtcgccgccgccgccggccggtagtagcggcgtgccacaggcggcgggaggcggcggtgtctcttggggcggcccccggttgcgcgccgaggacgagcacgccgtcatcgtcgcggcgctggctcacgtcgtcggcgccgggacgcggagctcgccaccggccgggctcggccagcaaggtacgtgcccacgtgctcgtggtgcacgggcccacgtgcagtgctccacactttttacctacagcggggtggatatgg

SEQ ID NO: 81 - DNA, 1538 bp, Zea mays
ggcggcggagaccgccggcgtcttgtccggtgagtaccaggccctcgagacgtccacgctggtgtcggctctggcccacgtcgtcgctagcggcggcgaagggtacccgccgtggtccggcgcccggcggggggatgacagtcgtcgtcctacggcaacaatggtggcgccggtcggcaccggcggccacggctactccgccgcaaccgcaccgacaccgccggctcacttcttcggagcaggttagcaagcacaccgctcctacgtgcaaatattctactgtacttgatttatttggagatcgcagcaaggcatccattccagtatcaaaaacaagttcgttaacttaaaacaaagaacagcgagctccgcgtgacgcttgtgcggctttacttgcgtccccctttaatttttttttatatatagttggggtctcttttcagaacggttgctgttcaattagcatgtgttagtcatgcgggcagaatcaaaagttggtgttagtgcggcatattaattatattgcttagatgaatatactttgcttgcgccaagacgaatgctacgtcgtcgtcagagcgaactaactgtccatgaatgaatgacatatagactaactgtccatgaatgaatgataaaattatttctgttttttttctcgaacaattttgaaaacatttttttccattcttgcattgttccagcgcgagatacagatcggtatggattaaagcgttactttcttcgtaatttggaaattgaactcgtcttgaaccaggattcacatgcattgccttttccatccagattcaatgcacaacgcccgaaaaatgtcgagaagaagcgacgactgcaatttgtgtaaaacatgcatggtgcacgtacgcaaatgagcgcatgtggatttcacttgtctcgccgattgaatcaaattaaagattaggcggcagccccatccacaccattttatttttttgaaaccgcgtacacgtctagaataaatacagccaaaaatatattgctacactgctacatgtaggatctattatcacgttcttagccgtgtcacgtcgttagctatgagatatcgctacagtgattagctagctaggtacaagagtcgtagagcggtagaggagaagcgtcgacgtacgccatcgtcacatgcatgcgtagcagagcacaggaagccgccaggaaggaaccctgctacgtacacgtacgtgtctacagctgcccatgcccgcatcgatacggataggtgcgcatttaatacaccaccaccacaacaaaaatacgacccgtatatgcatgggcatggcgcggacgccgacgcatggcgggtcaaacgcacgacgccgccgacgcactatatatccgtcgcactgcttaccctacgctacgtcgtgtccgtgtgtgccgtgtccgtgtgcgcgactagctagctagtgctgtgcacagtgtactcatctcatctctatctatccgcgcacgcaggaggagagcaggcggcccacgacgtgcagggcgcggcggcc

SEQ ID NO: 82 - DNA, 1200 bp, Zea mays
ctaaacagatctgatgtctgaagccagtgacgtatttattcatacacgacactaaggtctgtttagttgggttgtggctgtgaaaaaaactgttatgggctgtgagctgtgaaaaaacaaccgtgggttgtgggctgttaaaaaaaagctaaaactgtccggtgaaagccgataaaagttcttccatatatattttagagttccatttgaaaaccgctaaaagcaggtctagaggtgctttcaattttacaatacaagaaagtcggtttttagaaaaaagctgcttcctggatatagccctttggtttaactttttggcttttaagaagcaagagtcaaaggcaaaaactaaaccaaacacaccctaaacatatcgagcgtctgaaggcagtaatgtatatgtatttgtaacattaatttaaatacacgtcattaaacatagatatataaaataaaagacatcactaaattattaccatgttaccatattttactagtggcgcgtttctacaataggtgtcacgattttgacttgacgtagaggtcagtttggtttgatgcctaacttaccatattttacctattttttctgcctaaaattagttcttccattagagacgactaaccttaagcaaattatgatatatttagccgcgaaccaaacatttttactgctttccaccacgttgttattgttagtgctcgagtagaccgcaaatgcgaacattttcttcagaatctggaaattaagctcatcttctcatgcattgcacctccatcaaaattcaatacgtacgtacaccacgccggaaaatgtcgatcgatttgaccgagaagaagagattattgcaagtgtgtaaaacatgatgcatgcatgcgcaagcatgtggatttcaccttgtcaccgattgatccacaccttttccgccgctccaactcccaccaaacaatccgctagattacatacgctatgctgtagcgacgagatattgctacagtgattaggtagaagagaggggaggagacgccattgtaacatggatggacgaggacgacaggaagctagctggctggctacgtacacgtaggtatacgtatgtatgtacgtacgtgagacagcaaggctgcacctgtcccatgcccgcatcgatacggatagatgcgtatttattaccactacaacaaaaatacgacccgtat

SEQ ID NO: 83 - DNA, 1689 bp, Zea mays
gagatttaaatgtagacttgagaggatattcttattgtgtttcataatatcataagatggtgatttagataaggttccttagcttatctataataatggcaacgtgatcctttttttttaggagagggatattttaggttatgtttcatggtgacaggatagatgcttgcaatttagatatagagttaggatcatctatgttttttccataatgacaatgcagctgtgctttaagaaaacaaatatatatttcatagaacgatatatgttattatcaatgctgagtagtagtgtacatcaaattaatatactagatctttgtaattttctgaaaaatagaatctatttttatatcacgtcttataatttttttagctattagctaactaatagttaatgactttttagctattagcagacccttaaatatatattaatgttgtaatgtagaggcttcactaatctcaccaattagtaaaatattaccttattttttaaaattgtaataaatacttttagcttgtatatagatacatatagagccggttttataggctcacacctagaaattatagattttgatatatctagatatataggaataattgaaaaacaaagatattttatagcttaaaacaaaaggaatatacctacaaacagaaacaatcaaacaagcaaccactgaagtatgtcaagaccagtacctacgtactagcgtagttagcattcaggcatggtggttctcagcgccgatcgccgatcaatggatggttccagtagcaaatttgttattaagagcaagtttaataatagagttcattgttaactctaactccttatcatatcgtttagagttaataataaacatacttatataataaagctggctataagattgactcttatattattgttttgtctgttttcctttcattcttttatcttgtcacatgagagtctgggtcctctattttttatgtctcttttcttcgcataagaaaaatgctatataaatattaaacttattgtctatttgatctaagaaaaaaaaagttgatggtgccaattcacgtctcgccatctcccaagatatttgcagttgaaactagagtcgtagacacgtcacacaagcggtcaatagaccaaggacacgtcaggcccactccctccacgttttcactcctacgtggcagccacccagggaccaccccgtggatcacgcacccgatcacgcacgccctggcccacgcggacggccctggctccgccactgtggcggaaacggacggcatcggctaatggacaaacaaacggccggtagcgcgacacgtctcccctcgatgcgttttatccccacgatgcatccgtcgcgacgagcgctgcgccggtgcccccattctccactcggactccgctctcctgccccgctctccctcggtccctcccaggcccagcttccagctcgctctccgccgccgctgctatcccgctctctctcctctacctcctatcctcccagtcccagccagagctgtcctgctggttctctgcgtctgctgcgctcccgccgctgctgctcgtgaagttattccttcggagaggcagtgtgtttggatcgttttggtcagcctcctcggagttgtttgacttgaccggatcggacactgtttggtggctcaatcggccgcg

SEQ ID NO: 84 - DNA, 1480 bp, Zea mays
gcaaaacgcctagcatctcctaggcaagcctaggctggactagtctctaggctaagggtcatcgtctagattccgcctagcgtctagcaaaactttgtatgtgtctatatcaaaaaacaaataatcaatttaaatgaattgtataatcttagaccttgtttgtttattcccattctataggaattagaggggattagaagaaattaagaagctttttttgacttatactcaatcctctccaatccatatggattgagattaaaacaaacgggtccatagggaaatgaaccatatttgattgaattgattcttcacatatgtcatgtctgcattattgttatcctgttgcaacgagtgtgctgtgcatttacctagtatgtcttacggcaacgactgctatgcaagcagtccttctatacaaacgtgcaagcaaaccatatgatccattagatcgtgatttaaccgcaagtcacatttagcacttaaatccttccaccaccagctcaataatctttataaaaaaacccctaacaaatcatggttgtatctgtggttggatcgtaatctaatgaatcaaatagtttgcttgcacgcttacacagaaacactgcttgcatagcagtcgttgcctatgtcttacatgcaaatttcatgagtcatgggctcataaccaatgccactggtacactgttataaggaatattcgtcctagcgtaaagaacttaagtatattaatataatttaccttacacatctaaagaagcacaaatccattgaaaatgataaatttcagttcttaccttgtcctcgatctcatctgttttgattgctggtaaatacattcgatcctttttttaaaaaaaaactgaagatatttttgccacttcaatctattttgacgtgatcatacggaccgccaggcattttttttcgcctttctttaacctcatctcatcggcaattaaagaccagggaaatagtaattgcgacaggcttctctggtcctattggcgatcaggacaacccacctaaaacacatgccaaaaagggctttctctccctgctgaacaccaactaccctgcgccgggtccagggtgcgccgggttctccctctcgctcccacgcggcaaaccccacgacgtgctataaatattccacgaacggcccggatacctccagccccgcatcgcaccctccctggccgccttctcttctccagcgtccgatctctcccactcgccttcctcaccgcagctctcccggctcggtcgcttcgccacctccgtcctccccccgcgctcggtcgctcgccacctgctctcccctccctccacgttgctcgcgcccgcgcttatataaggtacgccgcctcgactctccaaacccctccgcgtggacctaaggtccggcgcgccgagatggagctgatggatctagggtttcggttgcggcggcggtcctgtagtgcaggaggagctc

SEQ ID NO: 85 - DNA, 1476 bp, Zea mays
gttgcagtacaatagaaatcaaatgctctaatctttagtcctggttgcacaaatcgatagtgaaggtaagcctttattgccgggttttggcttgaattggtactaaaagtccaactttagtatcgattcctggcatgaattggtactaaatagggaattttagtaccagtttgagcggtacaaaagtgactttttgttttacatatttataaatgatattgatgaatatttaatatatacacgtactaattaatatatgtacatataaaatgtacaagcaagtagttttttttcaaagggacatgtatataaatattaagggtagctctttccaataggacattaacacaaatatacaagtaaatagtatagtaatatatgtacaataatacatacaaatgaacagcaataatagttcttgaattgcttcaatcttttgcaaagagagtagttctttccttcatctttataaactatgatttcaaaaaaaagtattcttatattaaattcaaagctgaaatactttatcaaacccaaattaaacccaaagagagtagttcgtttctttcttcttcaagtgttcgtcacatcggcattgtacatatatacataaactttccttcctttcttcacgcttcacctttaactcggcactggtgaaaacattatgatagacggaactagcatggattacaacattaataataaatatacgctaaaacattctgatagataaagactgataatgtgatcaatagtacagacaatagttgatatgttgatcgataagagttaggtgcattagagcatctccaataataacaataatacctcaaactggtgtctcaaattgaaatataggactctacacagaaaaactactctaacgatgtcttattttataaaatttgattaaaacagtataaagcattttctcaagtgcctcaaatatattacaccatagtgacttccctataatatagatttatggttttaatgttgaagcagaatgttttttatgccataaattctataaattatatttatttttaaattataaagtatttttatgttatgctgttggagaaccactctcctagtgaacaccaactacccaaagagagtagttctccctctcgcacccacgcggcagcccccacggcgtgctataaatacttcacgaacggcccggatatctccatccctgcatcgcaccctcccgggccgccttctcttctccagcgtccgatctcccactcccctccctcaccgcagctctcccacctccgccctccccccgcacgcgctcgccacctcgccctcccctccacgttgctcgcacccgcgcttatataaggtatgcctcttgccctctccaaacccctccgcgagggcctagggtctggcgtgctgagctggagctgatggatctagggtttgggttgcggtgatggtcctgcagtgcaggaggagctc

SEQ ID NO: 86 - DNA, 1200 bp, Zea mays
accacgatcagcaagaacaatggcaccagcttcaagccatggcatcagcttgaattttatcaaaaaaaaaactgagacgaaatcgttgacaatttgtaataattcgatttttagatagcacaatttgaatggacatgattatattttgactgcaacactgtatttttagatagcacaatgttgtgtatttttagatagcacagtctacgtgctagcttgtttaactattgctgttatgcgcccatgtggacagcgtgagcccatggatttatactgattcattagtcgctggaggtgtttgcttgtttatgagggcccttggattgctaatgtggattggcccatggattttgaaaggatcggagcggattagggtgagaaggttttaaaatcgattcggattgagctacaacaaattattttggatttaggtagggccgagtcagcttctaagtcttctaggattggagtggggttagggctggattgtgacccattctcaggcctacgtcgggcgcgtgggttcctaagctgcttacttggctttggtgtggccgcgcggtgtgctgtgggctgtggcgttgccgtcttgggtttcgtcgcttggcgagcacggatgaggatcgaggaacgctcgaggcaggtgggtcagtcagtcgtgcgaccaaatacataaattaccatgatattatatcccattgcaacgcacgggtacttatctagtcaggtcattaaatgtaaatgaaatggcattgttggaagaaatccggtagaaataacatatcaagttctctaccaccgggtccactttcgtaaaacaaaaatagaagtcgaccgacaggagctagacgacgggcccatggccagggcccactcgctcttgttccctatttacagccgacgaacggcccagatacctccgcttccaccatcgcacccacccggagcgcctcctcttctccagcgtccgatccccattccccacctctcctccctccgccgccagctcccgcccccttctctcccctcctcgcctccccgcgcgcgcgtttttataaggtacgtacggcgcgtcgacacgctgcgcgcccgccaccttccgattcccggcggcacctctctacgatctgacgcgatcgatgacgtgtggcttcgtcttggtggatctagggtttcggcggaggcgcccggtcgctggcg

SEQ ID NO: 87 - protein, 40 aa, Zea mays
  NON_TER (1)..(1)
  misc_feature (3)..(3): Xaa can be any naturally occurring amino acid
  misc_feature (5)..(5): Xaa can be any naturally occurring amino acid
  misc_feature (11)..(11): Xaa can be any naturally occurring amino acid
  misc_feature (19)..(22): Xaa can be any naturally occurring amino acid
  misc_feature (30)..(32): Xaa can be any naturally occurring amino acid
  misc_feature (34)..(34): Xaa can be any naturally occurring amino acid
  misc_feature (37)..(38): Xaa can be any naturally occurring amino acid
  NON_TER (40)..(40)
RGXRXRPWGKXAAEIRDPXXXXRVWLGTFXXXEXAAXXYD

SEQ ID NO: 88 - protein, 56 aa, Zea mays
  NON_TER (1)..(1)
  misc_feature (5)..(5): Xaa can be any naturally occurring amino acid
  misc_feature (19)..(19): Xaa can be any naturally occurring amino acid
  misc_feature (30)..(30): Xaa can be any naturally occurring amino acid
  misc_feature (34)..(34): Xaa can be any naturally occurring amino acid
  misc_feature (37)..(37): Xaa can be any naturally occurring amino acid
  misc_feature (41)..(41): Xaa can be any naturally occurring amino acid
  misc_feature (45)..(45): Xaa can be any naturally occurring amino acid
  misc_feature (47)..(47): Xaa can be any naturally occurring amino acid
  misc_feature (49)..(50): Xaa can be any naturally occurring amino acid
  misc_feature (52)..(52): Xaa can be any naturally occurring amino acid
  NON_TER (56)..(56)
RGVRXRPWGKWAAEIRDPXKAARVWLGTFXTAEXAAXAYDXAALXFXGXXAXLNFP

SEQ ID NO: 89 - protein, 91 aa, Zea mays
  NON_TER (1)..(1)
  misc_feature (12)..(12): Xaa can be any naturally occurring amino acid
  misc_feature (18)..(18): Xaa can be any naturally occurring amino acid
  misc_feature (20)..(20): Xaa can be any naturally occurring amino acid
  misc_feature (24)..(24): Xaa can be any naturally occurring amino acid
  misc_feature (27)..(27): Xaa can be any naturally occurring amino acid
  misc_feature (30)..(30): Xaa can be any naturally occurring amino acid
  misc_feature (34)..(34): Xaa can be any naturally occurring amino acid
  misc_feature (45)..(45): Xaa can be any naturally occurring amino acid
  misc_feature (47)..(47): Xaa can be any naturally occurring amino acid
  misc_feature (55)..(55): Xaa can be any naturally occurring amino acid
  misc_feature (71)..(71): Xaa can be any naturally occurring amino acid
  misc_feature (77)..(77): Xaa can be any naturally occurring amino acid
  misc_feature (79)..(79): Xaa can be any naturally occurring amino acid
  misc_feature (84)..(84): Xaa can be any naturally occurring amino acid
  misc_feature (87)..(87): Xaa can be any naturally occurring amino acid
  misc_feature (89)..(90): Xaa can be any naturally occurring amino acid
  NON_TER (91)..(91)
REQDRFLPIANXSRIMKXAXPANXKIXKDXKETXQECVSEFISFXTXEASDKCQXEKRKTINGDDLLWAMXTLGFEXYXEPLKXYLXKXXE

SEQ ID NO: 90 - DNA, 20 bp, Zea mays
gtaaacaaatcggtgcttgc

SEQ ID NO: 91 - DNA, 20 bp, Zea mays
agtaatttcgggattcacga

SEQ ID NO: 92 - DNA, 20 bp, Zea mays
gtgtttcgaacgtaaactcg

SEQ ID NO: 93 - DNA, 20 bp, Zea mays
caacggcgacgaaacgagtg

(19) United States
(12) Patent Application Publication
(10) Pub. No.: US 2021/0139924 A1
(43) Pub. Date: May 13, 2021

(54) GENES AND GENE COMBINATIONS FOR ENHANCED CORN PERFORMANCE

(71) Applicant: YIELD10 BIOSCIENCE, INC., Woburn, MA (US)

(72) Inventors: Madana M.R. AMBAVARAM, Norwood, MA (US); Jihong TANG, Woburn, MA (US); Kristi D. SNELL, Belmont, MA (US); Oliver P. PEOPLES, Arlington, MA (US)

(21) Appl. No.: 17/044,015

(22) PCT Filed: Apr. 1, 2019
(86) PCT No.: PCT/US2019/025163

§ 371 Date: Sep. 30, 2020

Related U.S. Application Data
(60) Provisional application No. 62/669,662, filed on May 10, 2018.
(60) Provisional application No. 62/651,451, filed on Apr. 2, 2018.

(51) Int. Cl. C12N 15/82; C07K 14/415

(52) U.S. Cl. CPC C12N 15/8213; C12N 15/8247; C07K 14/415; C12N 15/8234

(57) ABSTRACT

The present invention identifies a number of transcription factors of corn, genes encoding the transcription factors, and methods to enhance characteristics of corn such as higher photosynthesis rates, higher photosynthetic electron transport rates, reduced photorespiration rates, higher biomass yield or content, higher seed yield, improved harvest index, higher oil content, improved nutritional composition, improved nitrogen use efficiency, drought resistance, flood resistance, disease resistance, salt tolerance, higher C02 assimilation rate, and lower transpiration rate in a plant by upregulating the genes encoding the transcription factors. Compositions of the invention comprise polypeptide sequences, polynucleotide sequences, variants, orthologs, and fragments thereof. Methods comprise introducing into corn plants systems that increase the expression or activity of transcription factors of the invention. Methods and compositions also provide corn plants with enhanced performance.

### FIELD OF THE INVENTION

The present invention relates generally to corn transcription factor gene targets, genetic engineering technologies, genome editing materials and methods for upregulating the expression of those gene targets alone or in combinations and more particularly, to corn plants having increased expression of those gene targets such that they have improved performance in soil as compared to the same plant having normal expression of those genes.

### BACKGROUND OF THE INVENTION

The world faces a major challenge in the next 35 years to meet the increased demands for food production to feed a growing global population, which is expected to reach 9 billion by the year 2050. Food output will need to be increased by up to 60% in view of the growing population.

Maize which is also known as corn together with wheat, rice and soybean provides nearly two thirds of global agricultural calories (Citation: Ray D K, Mueller N D, West P C, Foley J A (2013) Yield Trends Are Insufficient to Double Global Crop Production by 2050. PLoS ONE 8(6):e66428. doi:10.1371/journal.pone.0066428). In the United States alone in 2017 around 90 million acres of corn was planted with an annual harvest of around 1.6 billion bushels making it the most valuable food and feed crop. Corn seed genetically engineered for pest resistance and/or herbicide tolerance is the dominant value driver in the US seed sector. Increasing the field performance of corn and in particular grain yield is critical to addressing global food security and is a major objective of the global seed companies.

Since the beginning of genome sequencing, researchers have tested thousands of plant genes individually in corn using genetic engineering techniques to increase or decrease the level of activity of the target gene product. However, other than large numbers of patent applications, including a significant number of theoretical patent applications in the United States fisting tens of thousands of genes in patent Claims (for example, US 2005/0108791; US 2009/0158452; US 2011/0258734; US 2013/0074202; and US 2012/0017292), the vast majority of which are based purely on DNA sequence homology and with no experimental data, there has been really no significant technical breakthrough or commercial developments using this approach. The long lists of potential crop improvement benefits, together with the very long lists of potential genes for achieving such benefits, is illustrative of just how little is actually taught or reduced to practice regarding specific gene targets to improve crop performance in these applications and is analogous to pointing to a dictionary and indicating there is a great work of literature contained in it. In reality, and absent data to the contrary, probably greater than 99% of the gene sequences listed in these broad cases will have either no meaningful impact or possibly be detrimental to performance. Therefore the need to identify specific corn transcription factor genes for upregulation to significantly improve the performance of corn remains an unmet need.

In the late 1980's and early 1990's, genetic engineering of transgenic plants was used for the first time to develop crops which are herbicide tolerant, or pest or disease resistant, by introducing genes from the most readily available source at the time, microorganisms, to impart these new functionalities. Unfortunately, “transgenic plants” or “GMO crops” or “biotech traits” are not widely accepted in a number of different jurisdictions and are subject to a regulatory approval process which is very time consuming and prohibitively expensive. The current regulatory framework for transgenic plants results in significant costs (˜$136 million per trait; McDougall, P. 2011, The cost and time involved in the discovery, development, and authorization of a new plant biotechnology derived trait. Crop Life International, website: croplife.org/wp-content/uploads/pdf files/Getting-a-Biotech-Crop-to-Market-Phillips-McDougall-Study.pdf) and lengthy product development timelines that limit the number of technologies that are brought to market. These risks have severely impaired private investment and the adoption of innovation in this crucial sector. Recent changes in the regulations governing genetically modified crops by USDA-APHIS in the United States and new technologies such as genome editing have begun to change this situation. For example, a corn plant which has been genetically engineered to modify the activity of a corn gene using only DNA sequences from corn, technically described as cis-genic not transgenic, may be classified as non-regulated provided the engineered corn plant contains no foreign DNA sequences. Advances in genome editing technologies provide an opportunity to precisely remove or insert DNA sequences in the plant genome of interest to inactivate specific plant genes or to alter their expression by modifying their promoter sequences to improve plant performance (Belhaj, K. 2013, Plant Methods, 9, 39; Khandagale & Nadal, 2016, Plant Biotechnol Rep, 10, 327). Genetically engineered plants made using this approach contain no foreign DNA sequences and may also be categorized as non-regulated by USDA-APHIS. In both cases however, the regulatory status of the engineered plants are appropriately subject to the usual criteria for approval of any new plant variety.

Clearly there is a need in corn to identify specific transcription factors whose expression can be modified using only corn DNA sequences alone or in combinations to improve corn crop performance.

### BRIEF SUMMARY OF THE INVENTION

It is an objective of this invention to provide specific transcription factor genes for corn as well as the methods, DNA and RNA sequences for modifying or editing these transcription factor genes to increase their expression or activity and improve the performance of corn plants. It is a further objective of this invention to provide corn plants, which have been modified according to this invention and which have improved performance characteristics in the field as compared to the same corn before it was modified as disclosed herein.

Accordingly, provided herein is a method for modifying a corn plant, the method comprising upregulating, in the corn plant, one or more polynucleotides or polypeptides selected from among the following:

(a) one or more polypeptides comprising SEQ ID NOS: 87, 88, or 89;

(b) one or more polypeptides comprising SEQ ID NOS: 4, 6, 8, 10, 14, 16, 18, 20, 24, 26, 28, 30, 32, 41, 43 or 48;

(c) one or more of the polypeptides set forth in (a) having at least 85%, 90%, 95% or higher sequence identity to one or more of the polypeptides set forth in (b);

(d) one or more polynucleotides comprising SEQ ID NOS: 3, 5, 7, 9, 13, 15, 17, 19, 23, 25, 27, 29, 31, 40, 42 or 47;

(e) one or more polynucleotides having at least 85%, 90%, 95% or higher sequence identity to one or more of the polynucleotides set forth in (d); or

(f) one or more polypeptides encoded by one or more of the polynucleotides set forth in (d) or (e).

In accordance with the method, the one or more upregulated polypeptides can be transcription factors. Also in accordance with the method, the one or more upregulated polynucleotides can encode transcription factors.

In various aspects of the method, the one or more upregulated polynucleotides or polypeptides exhibit at least a change in expression or at least a two-fold change in expression as compared to that of a control plant.

In certain aspects, the expression of the transcription factor gene is upregulated using traditional genetic engineering techniques such that one or more additional copies of the transcription factor gene is inserted into the corn genome under the transcriptional control of promoters which are heterologous to the transcription factor gene. Such recombinant or chimeric gene constructs are well known in the art. Preferably the method of introducing the additional copy of the transcription factor gene does not introduce any non-corn or foreign DNA sequences, or where any foreign DNA sequences used during the process of constructing the modified corn plant are subsequently removed.

In certain aspects, the expression of the transcription factor gene is accomplished by deletion, insertion and/or substitution of one or more nucleotides to increase gene expression using gene editing techniques using a CRISPR nuclease selected from Cas nuclease, Cas9 nuclease, CasX nuclease, CasY nuclease, a Cpf1 nuclease, a C2c1 nuclease, a C2c2 nuclease (Cas13a nuclease), NgAgo nuclease, or a C2c3 nuclease. For instance, one or more polynucleotide sequences can be upregulated by targeting a guide polynucleotide to a target site selected from a promoter, a promoter element, a terminator or a coding sequence of the polynucleotide sequence using a CRISPR/Cas system to form a complex suitable for editing a corn genome. Alternatively, transcription activator-like effector nucleases (TALENs) or zinc finger nuclease (ZFN) techniques can be used for editing instead of a CRISPR nuclease.

The methods can be used to produce modified corn plants exhibiting one or more enhanced characteristics selected from higher photosynthesis rates, higher photosynthetic electron transport rates, higher non-photochemical quenching, reduced photorespiration rates, higher biomass yield or content, higher seed yield, improved harvest index, higher seed oil content, improved nutritional composition, improved nitrogen use efficiency, drought resistance, flood resistance, disease resistance, salt tolerance, higher CO₂ assimilation rate, or lower transpiration rate

Also provided herein is a modified corn plant comprising:

(a) one or more polypeptides comprising SEQ ID NOS: 87, 88, or 89;

(b) one or more polypeptides comprising SEQ ID NOS: 4, 6, 8, 10, 14, 16, 18, 20, 24, 26, 28, 30, 32, 41, 43 or 48;

(c) one or more of the polypeptides set forth in (a) having at least 85%, 90%, 95% or higher sequence identity to one or more of the polypeptides set forth in (b);

(d) one or more polynucleotides comprising SEQ ID NOS: 3, 5, 7, 9, 13, 15, 17, 19, 23, 25, 27, 29, 31, 40, 42 or 47;

(e) one or more polynucleotides having at least 85%, 90%, 95% or higher sequence identity to one or more of the polynucleotides set forth in (d); or

(f) one or more polypeptides encoded by one or more of the polynucleotides set forth in (d);

wherein the one or more polypeptides of (a), (b), (c), or (f) or the one or more polynucleotides of (d) or (e) are upregulated.

In accordance with the modified corn plant, the one or more upregulated polypeptides can be transcription factors. Also in accordance with the modified corn plant, the one or more upregulated polynucleotides can encode transcription factors.

In various aspects of the modified corn plant, the one or more upregulated polynucleotides or polypeptides exhibit at least a change in expression or at least a two-fold change in expression as compared to that of a control plant.

Again, in some embodiments the expression of the transcription factor gene is upregulated using traditional genetic engineering techniques such that one or more additional copies of the gene is inserted into the corn genome under the transcriptional control of corn promoters which are heterologous to the transcription factor gene. Such recombinant or chimeric gene constructs are well known in the art. Preferably the method of introducing the additional copy of the transcription factor gene does not introduce any non-corn or foreign DNA sequences or where any foreign DNA sequences are subsequently removed.

In some embodiments, the polynucleotide sequence encoding one or more transcription factors are upregulated by DNA insertion, deletion, insertion and/or substitution of one or more nucleotides, site-specific mutagenesis, chemical mutagenesis, using gene editing techniques such as CRISPR nuclease selected from Cas nuclease, Cas9 nuclease, CasX nuclease, CasY nuclease, a Cpf1 nuclease, a C2c1 nuclease, a C2c2 nuclease (Cas13a nuclease), a C2c3 nuclease, or a NgAgo nuclease, or by using TALEN or ZFN techniques. For instance, one or more polynucleotide sequence can be upregulated by targeting a guide polynucleotide to a target site selected from a promoter, a terminator or a coding sequence of the polynucleotide sequence using a CRISPR/Cas system to form a complex suitable for editing a plant.

Compositions useful for overexpression of a transcription factor using transgenic or cis-genic technologies described herein are also disclosed.

The compositions include a recombinant nucleic acid molecule comprising:

(a) one or more polynucleotides comprising SEQ ID NOS: 3, 5, 7, 9, 13, 15, 17, 19, 23, 25, 27, 29, 31, 40, 42 or 47;

(b) one or more polynucleotides having at least 85%, 90%, 95% or higher sequence identity to one or more of the polynucleotides set forth in (a); or

(c) a fragment of any one of the polynucleotides set forth in (a) or (b) that regulates gene expression; and

further comprising a polynucleotide heterologous to the one or more polynucleotides of (a) or (b) or the one or more fragments of (c).

The compositions also include a recombinant polypeptide molecule comprising:

(a) one or more polypeptides comprising SEQ ID NOS: 87, 88, or 89;

(b) one or more polypeptides comprising SEQ ID NOS: 4, 6, 8, 10, 14, 16, 18, 20, 24, 26, 28, 30, 32, 41, 43 or 48;

(c) one or more of the polypeptides set forth in (a) having at least 85%, 90%, 95% or higher sequence identity to one or more of the polypeptides set forth in (b); or

(d) one or more fragments of any one of the polypeptides set forth in (a), (b), or (c) that regulates gene expression; and

further comprising a polypeptide heterologous to the one or more polypeptides of (a), (b), or (c) or the one or more fragments of (d).

The compositions also include a recombinant nucleic acid molecule comprising: (a) a promoter sequence functional in corn, operably linked to (b) a polynucleotide selected from SEQ ID NOS: 3, 5, 7, 9, 13, 15, 17, 19, 23, 25, 27, 29, 31, 40, 42 or 47 encoding a transcription factor gene operably linked to (c) a terminator sequence functional in corn.

The compositions also include the following:

(a) one or more polynucleotides encoding one or more of SEQ ID NOS: 52-55 or one or more of SEQ IDS NOS: 75-86 that regulate the expression of the transcription factor genes encoded by SEQ ID NOS: 3, 5, 7, 9, 13, 15, 17, 19, 23, 25, 27, 29, 31, 40, 42 or 47 in corn; or

(b) a DNA construct targeting the one or more polynucleotides encoding one or more of SEQ ID NOS: 52-55 or one or more of SEQ IDS NOS: 75-86 that comprises:
(i) an expression cassette for a polynucleotide sequence encoding a CRISPR nuclease containing a promoter sequence functional in corn; operably linked to a CRISPR nuclease with codon usage appropriate for use in corn that is flanked by nuclear localization sequences (NLS) to ensure delivery of the enzyme into the nuclei; and flanked at the 3′ end by a terminator sequence functional in corn;
(ii) one or more expression cassettes for one or more sgRNAs to direct the CRISPR nuclease to the appropriate desired nuclease cut site(s), each cassette containing: a promoter sequence functional in corn that is appropriate for the expression of sgRNAs (i.e. plant, and preferably monocot, RNA polymerase III promoters, such as U6 and U3); DNA encoding an RNA guide sequence of ˜20 nucleotides; DNA encoding a guide RNA scaffold (gRNA Sc) which when combined with the previously described RNA guide sequence forms a functional sgRNA; and a poly T-termination signal;
(iii) a promoter replacement cassette to be inserted in the double stranded break created by the Cas nuclease at the sgRNA target sequence(s) containing: a DNA fragment homologous to the genomic DNA region flanking the 5′ double stranded break site; the promoter to be inserted; and a DNA fragment homologous to the genomic DNA region flanking the 3′ double stranded break site; where the homologous regions direct the insertion of the new promoter fragment by the plant's endogenous repair mechanisms; or
(iv) an expression cassette for a selectable marker containing: a promoter sequence functional in corn, operably linked to a selectable marker appropriate for corn, flanked by a poly T-termination signal.

Such DNA constructs can provide for enhanced characteristics selected from higher photosynthesis rates, higher photosynthetic electron transport rates, higher non-photochemical quenching, reduced photorespiration rates, higher biomass yield or content, higher seed yield, improved harvest index, higher seed oil content, improved nutritional composition, improved nitrogen use efficiency, drought resistance, flood resistance, disease resistance, salt tolerance, higher CO₂ assimilation rate, or lower transpiration rate.

Exemplary embodiments include the following.

Embodiment 1: A method for modifying a corn plant, the method comprising upregulating, in the corn plant, one or more polynucleotides or polypeptides selected from among the following:

(a) one or more polypeptides comprising SEQ ID NOS: 87, 88, or 89;

(b) one or more polypeptides comprising SEQ ID NOS: 4, 6, 8, 10, 14, 16, 18, 20, 24, 26, 28, 30, 32, 41, 43 or 48;

(c) one or more of the polypeptides set forth in (a) having at least 85%, 90%, 95% or higher sequence identity to one or more of the polypeptides set forth in (b);

(d) one or more polynucleotides comprising SEQ ID NOS: 3, 5, 7, 9, 13, 15, 17, 19, 23, 25, 27, 29, 31, 40, 42 or 47;

(e) one or more polynucleotides having at least 85%, 90%, 95% or higher sequence identity to one or more of the polynucleotides set forth in (d); or

(f) one or more polypeptides encoded by one or more of the polynucleotides set forth in (d) or (e).

Embodiment 2: The method of embodiment 1, further comprising growing the modified plant under conditions whereby the modified plant exhibits one or more enhanced characteristics as compared to a control plant grown under similar conditions.

Embodiment 3: The method of embodiment 1 or embodiment 2, wherein the one or more upregulated polynucleotides comprise SEQ ID NOS: 3, 5, 7, 9, 13, 15, 17, 19, 23, 25, 27, 29, 31, 40, 42 or 47.

Embodiment 4: The method of any one of embodiments 1-3, wherein the one or more upregulated polynucleotides or polypeptides exhibit at least a change in expression or at least a two-fold change in expression as compared to that of a control plant.

Embodiment 5: The method of embodiment 4, wherein the change in expression is accomplished by introducing a transgene for one or more global transcription factors, wherein the transgene comprises a polynucleotide selected from SEQ ID NOS: 3, 5, 7, 9, 13, 15, 17, 19, 23, 25, 27, 29, 31, 40, 42 or 47.

Embodiment 6: The method of any one of embodiments 1-5, wherein the one or more upregulated polynucleotides or polypeptides are upregulated by insertion and/or substitution of one or more nucleotides, site-specific mutagenesis, chemical mutagenesis, targeting induced local lesions in genomes (TILLING), gene editing techniques using CRISPR nuclease selected from Cas nuclease, Cas9 nuclease, CasX nuclease, CasY nuclease, a Cpf1 nuclease, a C2c1 nuclease, a C2c2 nuclease (Cas13a nuclease), or a C2c3 nuclease, NgAgo nuclease, TALEN or ZFN techniques.

Embodiment 7: The method of embodiment 6, wherein the one or more upregulated polynucleotides or polypeptides are upregulated by targeting one or more guide polynucleotides to one or more target sites selected from a promoter, a terminator, or a coding sequence of the one or more polynucleotides set forth in (d) or (e).

Embodiment 8: The method of any one of embodiments 1-7, wherein the modified plant exhibits one or more enhanced characteristics selected from higher photosynthesis rates, higher photosynthetic electron transport rates, higher non-photochemical quenching, reduced photorespiration rates, higher biomass yield or content, higher seed yield, improved harvest index, higher seed oil content, improved nutritional composition, improved nitrogen use efficiency, drought resistance, flood resistance, disease resistance, salt tolerance, higher CO₂ assimilation rate, or lower transpiration rate.

Embodiment 9: The method of embodiment 8, wherein the modified plant exhibits an increase in seed oil content or seed yield as compared to a control plant.

Embodiment 10: The method of embodiment 9, wherein the seed oil content of the modified plant is increased by 10%, 20%, 30%, 40%, 50%, 60%, 70%, 80%, 90%, 100% or higher relative to the control plant.

Embodiment 11: The method of any one of embodiments 8-10, wherein the modified plant exhibits an increase in photosynthetic electron transport rate as compared to a control plant.

Embodiment 12: The method of embodiment 11, wherein the photosynthetic electron transport rate of the modified plant is increased by 5%, 10%, 20%, 30%, 40%, 50%, 60%, 70%, 80%, 90%, 100% or higher relative to the control plant.

Embodiment 13: A modified corn plant comprising:

(a) one or more polypeptides comprising SEQ ID NOS: 87, 88, or 89;

(b) one or more polypeptides comprising SEQ ID NOS: 4, 6, 8, 10, 14, 16, 18, 20, 24, 26, 28, 30, 32, 41, 43 or 48;

(c) one or more of the polypeptides set forth in (a) having at least 85%, 90%, 95% or higher sequence identity to one or more of the polypeptides set forth in (b);

(d) one or more polynucleotides comprising SEQ ID NOS: 3, 5, 7, 9, 13, 15, 17, 19, 23, 25, 27, 29, 31, 40, 42 or 47;

(e) one or more polynucleotides having at least 85%, 90%, 95% or higher sequence identity to one or more of the polynucleotides set forth in (d); or

(f) one or more polypeptides encoded by one or more of the polynucleotides set forth in (d);

wherein the one or more polypeptides of (a), (b), (c), or (f) or the one or more polynucleotides of (d) or (e) are upregulated.

Embodiment 14: The modified plant of embodiment 13, wherein the modified plant exhibits one or more enhanced characteristics as compared to a control plant grown under similar conditions.

Embodiment 15: The modified plant of embodiment 13 or embodiment 14, wherein the one or more upregulated polynucleotides or polypeptides exhibit at least a change in expression or at least a two-fold change in expression as compared to that of a control plant.

Embodiment 16: The modified plant of embodiment 15, wherein the change in expression is accomplished by introducing a transgene for one or more global transcription factors, wherein the transgene comprises a polynucleotide selected from SEQ ID NOS: 3, 5, 7, 9, 13, 15, 17, 19, 23, 25, 27, 29, 31, 40, 42 or 47.

Embodiment 17: The modified plant of any one of embodiments 13-16, wherein the one or more upregulated polynucleotides or polypeptides are upregulated by insertion and/or substitution of one or more nucleotides, site-specific mutagenesis, chemical mutagenesis, targeting induced local lesions in genomes (TILLING), gene editing techniques using CRISPR nuclease selected from Cas nuclease, Cas9 nuclease, CasX nuclease, CasY nuclease, a Cpf1 nuclease, a C2c1 nuclease, a C2c2 nuclease (Cas13a nuclease), or a C2c3 nuclease, NgAgo nuclease, TALEN or ZFN techniques

Embodiment 18: The modified plant of embodiment 17, wherein the one or more upregulated polynucleotides or polypeptides are upregulated by targeting one or more guide polynucleotides to one or more target sites selected from a promoter, a terminator, or a coding sequence of the one or more polynucleotides set forth in (d) or (e).

Embodiment 19: The modified plant of any one of embodiments 13-18, wherein the modified plant comprises one or more enhanced characteristics selected from higher photosynthesis rates, higher photosynthetic electron transport rate, higher non-photochemical quenching, reduced photorespiration rates, higher biomass yield or content, higher seed yield, improved harvest index, higher seed oil content, improved nutritional composition, improved nitrogen use efficiency, drought resistance, flood resistance, disease resistance, salt tolerance, higher CO₂ assimilation rate, or lower transpiration rate.

Embodiment 20: The modified plant of embodiment 19, wherein the modified plant exhibits an increase in seed oil content or seed yield as compared to a control plant.

Embodiment 21: The modified plant of embodiment 20, wherein the seed oil content of the modified plant is increased by 10%, 20%, 30%, 40%, 50%, 60%, 70%, 80%, 90%, 100% or higher relative to the control plant.

Embodiment 22: The modified plant of any one of embodiments 19-21, wherein the modified plant exhibits an increase in photosynthetic electron transport rate as compared to a control plant.

Embodiment 23: The modified plant of embodiment 22, wherein the photosynthetic electron transport rate of the modified plant is increased by 5%, 10%, 20%, 30%, 40%, 50%, 60%, 70%, 80%, 90%, 100% or higher relative to the control plant.

Embodiment 24: A recombinant nucleic acid molecule comprising:

(a) one or more polynucleotides comprising SEQ ID NOS: 3, 5, 7, 9, 13, 15, 17, 19, 23, 25, 27, 29, 31, 40, 42 or 47;

(b) one or more polynucleotides having at least 85%, 90%, 95% or higher sequence identity to one or more of the polynucleotides set forth in (a); or

(c) a fragment of any one of the polynucleotides set forth in (a) or (b) that regulates gene expression; and

further comprising a polynucleotide heterologous to the one or more polynucleotides of (a) or (b) or the one or more fragments of (c).

Embodiment 25: A recombinant polypeptide molecule comprising:

(a) one or more polypeptides comprising SEQ ID NOS: 87, 88, or 89;

(b) one or more polypeptides comprising SEQ ID NOS: 4, 6, 8, 10, 14, 16, 18, 20, 24, 26, 28, 30, 32, 41, 43 or 48;

(c) one or more of the polypeptides set forth in (a) having at least 85%, 90%, 95% or higher sequence identity to one or more of the polypeptides set forth in (b); or

(d) one or more fragments of any one of the polypeptides set forth in (a), (b), or (c) that regulates gene expression; and

further comprising a polypeptide heterologous to the one or more polypeptides of (a), (b), or (c) or the one or more fragments of (d).

### BRIEF DESCRIPTION OF THE DRAWINGS

FIG. 1 shows a CLUSTAL O(1.2.4) multiple sequence alignment of the switchgrass STR1 transcription factor (SEQ ID NO: 2) and its maize orthologs. SEQ IDs of proteins in alignment are as follows: GRMZM2G142179 (SEQ ID NO: 32); GRMZM2G018984 (SEQ ID NO: 10); Pavir.Ib00526 (SEQ ID NO: 36); Pavir.Ba00410 (SEQ ID NO: 2); Pavir.Bb03337 (SEQ ID NO: 33); GRMZM2G171179 (SEQ ID NO: 8); Pavir.J104875 (SEQ ID NO: 51); Pavir.Aa00281 (SEQ ID NO: 35); GRMZM2G018398 (SEQ ID NO: 4); and GRMZM2G110333 (SEQ II) NO: 6).

FIG. 2 shows a CLUSTAL O(1.2.4) multiple sequence alignment of switchgrass SUFI transcription factor (SEQ ID 12) and its maize orthologs. SEQ IDs of proteins in alignment are as follows: Pavir.Aa02595 (SEQ ID NO: 12); GRMZM2G016434 (SEQ ID NO: 14); GRMZM2G457562 (SEQ ID NO: 41); GRMZM2G100727 (SEQ ID NO: 43); Pavir.J04335.1 (SEQ II) NO: 39); GRMZM2G309731 (SEQ II) NO: 20); Pavir.Gb01735.1 (SEQ ID NO: 38); GRMZM2G087059 (SEQ ID NO: 16); and GRMZM2G425798 (SEQ ID NO: 18).

FIG. 3 shows a CLUSTAL O(1.2.4) multiple sequence alignment of switchgrass BMY1 transcription factor (SEQ ID 22) and its maize orthologs. SEQ IDs of proteins in alignment are as follows: Pavir.J05081 (SEQ ID NO: 22); Pavir.Ba00451 (SEQ II) NO: 44); GRMZM2G384528 (SEQ ID NO: 24); GRMZM2G180947 (SEQ ID NO: 26); Pavir.Ib01924 (SEQ ID NO: 45); Pavir.Eb03638 (SEQ ID NO: 49); GRMZM2G303465 (SEQ II) NO: 48); Pavir.J02009 (SEQ II) NO: 46); Pavir.J02756 (SEQ II) NO: 50); GRMZM2G064426 (SEQ ID NO: 28); and GRMZM5G804893 (SEQ ID NO: 30).

FIG. 4 illustrates the expression pattern of select maize orthologs of the switchgrass transcription factors STR1, STIF1, and BMY1 in maize. A.-C. In silico analysis of the expression pattern of genes for the maize orthologs of (A) STR1 (GRMZM2G110333, SEQ ID NO: 5), (B) STIF1 (GRMZM2G016434, SEQ ID NO: 13), and (C) BMY1 (GRMZM2G384528, SEQ ID NO: 23) in different organs and developmental stages in maize. Data was retrieved from the maize Electronic Fluorescent Pictograph browser (website: bar.utoronto.ca/efp_maize/cgi-bin/efpWeb.cgi). Levels of expression signals are in FPKM units (Fragment Per Kilobase of exon per Million fragments mapped). FPKM estimates the relative transcript abundance of each gene by combining the expression of all the transcripts of a gene. D. Expression analysis of the maize orthologs using RT-PCR. The levels of expression of the maize putative functional orthologs in different organs at different developmental stages of greenhouse grown maize plants (inbred line B73) were analyzed.

FIG. 5 illustrates expression cassettes for overexpression of the GRMZM2G384528 gene (SEQ ID NO: 23), a maize ortholog of the switchgrass BMY1 (SEQ ID NO: 21) transcription factor. (A) An expression cassette for YTEN26 (SEQ ID NO: 66) containing the hybrid maize cab-m5 promoter fused to the maize hsp70 intron (SEQ ID NO: 64); the maize GRMZM2G384528 transcription factor gene; and the maize hsp70 terminator. (B) An expression cassette for YTEN27 (SEQ ID NO: 67) containing the maize MADS-box promoter (SEQ ID NO: 56); the maize GRMZM2G384528 transcription factor gene; and the maize hsp70 terminator; (C) An expression cassette for YTEN28 (SEQ ID NO: 68) containing the maize trpA promoter (SEQ ID NO: 74); the maize GRMZM2G384528 transcription factor gene; and the maize hsp70 terminator; (D) An expression cassette for YTEN29 (SEQ ID NO: 69) containing the maize ubiquitin promoter and the maize ubiquitin intron (SEQ ID NO: 65); the maize GRMZM2G384528 transcription factor gene; and the maize hsp70 terminator.

FIG. 6 illustrates genetic components at different stages of the Cas enzyme mediated genome editing process using the Cas9 enzyme as an example. Delivery of the genetic components can be achieved in multiple ways. Genetic transformation can be used to deliver the expression construct depicted in (A) into a plant cell. Transcription of (A) will produce the single guide RNA (sgRNA) depicted in (B). The sgRNA will complex with the Cas9 enzyme (that is delivered separately through genetic transformation or other means) and achieve the structure depicted in (C) to promote cleavage of the target genomic DNA at the “guide target sequence”. Alternatively, the sgRNA (B) can be synthesized in vitro and introduced into cells, often in the form of Ribonucleoprotein complexes (RNPs) that contain Cas9 protein to produce the structure depicted in (C) to promote cleavage of the target genomic DNA at the “guide target sequence”. When using plant transformation techniques, the expression cassette (A) for production of the sgRNA is composed of a promoter, often a plant RNA polymerase III promoter, DNA encoding the “guide” of the sgRNA, DNA encoding a “guide RNA scaffold” (gRNA Sc), and a poly T-termination signal. The combination of the “guide” and the “guide RNA scaffold” are necessary to form a functional sgRNA. The DNA encoding the guide portion of the sgRNA in (A) is often identical to the “guide target sequence” of the genomic DNA to be cut in (C), however several mismatches, depending on their position, can be tolerated and still promote double stranded DNA cleavage. The guide portion of the sgRNA pairs with this complementary DNA sequence to be mutated (referred to as guide target sequence #3 in figure) that is adjacent to a 3′ protospacer adjacent motif (PAM) (C), an additional requirement for target recognition, and double stranded DNA cleavage occurs. When using the Cas9 enzyme for cleavage, all guide target sequences are typically ˜20-nucleotides adjacent to a 3′ PAM sequence of (NGG) to initiate cleavage by the Cas9 enzyme. When using the CpfI enzyme for cleavage, guide target sequences are typically ˜23 nucleotides adjacent to a 5′ PAM sequence that varies with the specific enzyme. PAM sequences for select CpfI enzymes including engineered variants are shown in TABLE 7.

FIG. 7 illustrates the strategy for promoter replacement to change the expression pattern of a transcription factor using CRISPR genome editing. A. Guide target sequences (˜20 nt) in genomic DNA that are adjacent to a 3′ PAM sequence of (NGG) are identified in the region of the endogenous promoter to be replaced. DNA cassettes encoding sgRNA (See FIG. 6) are designed to bind the genomic DNA at the identified guide target sequences to promote DNA cleavage and excision of the promoter DNA. The general numbering strategy used for the promoter region for identifying guide target sequences is as follows. The sequence of the 5′UTR of the gene of interest plus at least an additional 1000 bp was analyzed for guide target sequences adjacent to a PAM site to target portions of the promoter region for excision. This genomic DNA sequence is given a SEQ ID number in TABLE 5 and TABLE 6. Since the length of the 5′ UTR varies for each gene, x denotes the size of the known or predicted 5′ UTR. Position #(1000+x) is the base directly in front of the ATG at the start of the coding sequence. In this example, guide target sequences identified for the design of three different sgRNAs are depicted in the promoter region. Pairs of sgRNAs can be used to excise regions of the promoter DNA for insertion of the new promoter replacement cassette, or alternatively, one sgRNA can be used. B. Cassettes for delivery into plant cells to achieve promoter replacement include i. a cassette to deliver the new promoter flanked by regions homologous to each side of the nuclease cut site [left and right flanking regions in (B), the flanking regions can additionally be flanked by guide target sequences and an adjacent PAM site to promote release of the cassette by Cas9 from a construct or other DNA]; ii. an expression cassette for the Cas9 nuclease or other site specific nuclease; and iii. an expression cassette(s) for DNA encoding sgRNAs to target cut sites that excise a portion or the whole promoter region. These cassettes can be transformed into the plant separately or on the same DNA through a variety of plant transformation methods including protoplast transformation, particle bombardment, nanotube or nanoparticle mediated DNA delivery (Kwak et al., 2019, Nature Nanotechnology, DOI 10.1038/s41565-019-0375-4) (Demirer et al, 2019, Nature Nanotechnology, DOI 10.1038/s41565-019-0382-5), and Agrobacterium-mediated transformation. The sgRNAs initiate a Cas9-induced double stranded DNA cleavage at the guide target sequence (or sgRNA binding site) in (A), whose sequence is complementary to the guide portion of the sgRNA. The regions of the promoter insertion cassette homologous to each side of the nuclease cut site direct the cassette's insertion into genomic DNA through the plants endogenous homology directed repair mechanism. C. Alternatively, CRISPR mediated promoter replacement can be achieved through the use of Ribonucleoprotein complexes (RNPs), The RNPs are created from a promoter insertion cassette, purified Cas9 enzyme, and synthesized sgRNA1 and sgRNA3 molecules. RNPs can be created and transformed into protoplasts as previously described by Woo et al., Nature Biotechnology, 2015, 33, 1162-1164. Nanoparticles or nanotubes capable of delivering biomolecules to plants can also be used (for review see Cunningham, 2018, Trends Biotechnol., 36, 882). D. Structure of the edited plant genomic DNA containing the new heterologous promoter inserted at the positions of Guide target sequences #1 and #3, that is created through (B) genetic transformation of cassettes or (C) delivery of RNPs,

FIG. 8 illustrates the plasmid maps for insertion of a heterologous maize promoter in front of the GRMZM2G384528 (SEQ ID NO: 23) gene, a maize ortholog of the switchgrass BMY1 transcription factor, using CRISPR Cas mediated promoter insertion through homology directed repair. Constructs are as follows: (A) binary construct YTEN30 (SEQ ID NO: 71) for Agrobacterium-mediated transformation to deliver the maize ubiquitin promoter and maize ubiquitin intron (SEQ ID NO: 70), (B) construct YTEN31 (SEQ ID NO: 72), a non-binary construct for transformation by protoplast transfection or particle bombardment to deliver the maize ubiquitin promoter and maize ubiquitin intron (SEQ ID NO: 70), and (C) DNA fragment YTEN32 (SEQ ID NO: 73) for delivery of the maize ubiquitin promoter and maize ubiquitin intron (SEQ ID NO: 70) to plant cells in ribonucleoprotein complexes (RNPs). (A) The YTEN30 construct contains a double enhanced CaMV 35S promoter driving the expression of a gene expressing Cas9 which has been codon optimized for rice. The gene encoding Cas9 is flanked by nuclear localization sequences (NLS) to ensure delivery into nuclei. The rice codon-optimized Streptococcus pyrogenes Cas9 and NLS sequence were synthesized using sequences described by Shan et al., 2013, Nat Biotechnol, 3, 686-688. A Cauliflower Mosaic Virus (CaMV) terminator sequence is downstream of the gene encoding Cas9. DNA fragments encoding two guides are fused to DNA encoding the guide RNA scaffold (gRNA Sc) to encode two separate functional sgRNAs. The DNA fragments are labeled Guide #1 and Guide #3 in the map and are equivalent to Guide target sequences #1 and #3 for GRMZM2G384528 in TABLE 5 whose positions within the promoter region are shown in FIG. 7. The resulting sgRNAs, produced upon expression of the DNA encoding the guide and gRNA Sc fragments from the rice U6 promoter (OsU6-2p), are designed to bind to the complementary guide target sequence on the genomic DNA and excise the promoter region of GRMZM2G384528. A poly T-termination signal is located downstream of the DNA encoding each sgRNA. A cassette containing the promoter to be inserted in the double stranded break created by the Cas9 nuclease near the PAM sites adjacent to guide target sequences #1 and #3 contains the following elements: DNA corresponding to the Guide #3 target sequence and its associated PAM sequence (labeled BMY1-3); a DNA fragment (˜800 bp in length) that is homologous to the region flanking the left side of the genomic DNA cut site (labeled HR-L); the maize ubiquitin promoter sequence with an intron (SEQ ID 70); a DNA fragment (˜800 bp in length) that is homologous to the region flanking the right side of the genomic DNA cut site (labeled HR-R); and DNA corresponding to the Guide #1 target sequence and its associated PAM sequence (labeled BMY1-1). The homologous regions flanking the promoter to be inserted enable insertion of the fragment into the plant's genomic DNA by the plant's homology directed repair mechanism. An expression cassette for selection of transgenic plants is included in the vector and contains a double enhanced CaMV35S promoter, an hsp70 intron, a hptI gene encoding hygromycin phosphotransferase containing an intron from the bean catalase-1 gene (CAT-1 intron), and a CaMV35S polyA sequence to provide hygromycin resistance to transgenic plants. The T-DNA sequence for insertion into the plant by Agrobacterium-mediated transformation is flanked by T-DNA left and right border sequences. (B) The YTEN31 construct is similar to YTEN30, except it is not a binary vector and does not have T-DNA border sequences. (C) The YTEN 32 fragment contains only the promoter insertion cassette of vectors YTEN30 and YTEN31. It is intended for use in RNPs with purified Cas9 enzyme and synthesized sgRNAs to cleave at the Guides #1 and #3 target sequences in the genomic DNA.

FIG. 9 illustrates cassettes for insertion into the genome at a Cas nuclease cleavage site to modulate the level of expression of the transcription factor. A. Schematic of the plant genomic DNA to be modified showing the positioning of three guide target DNA sequences (a, b, and c). The guide target sequences are adjacent to PAM sequences. B. Cassettes to be inserted to modulate expression of the transcription factor can be selected from one or more of the following: i. an expression cassette for a second copy of a transcription factor of interest containing a heterologous promoter (designated promoter x), the coding sequence (CDS) of the transcription factor, and a 3′ UTR (designated 3′UTR X). In this example, the insertion of this cassette is targeted to a genomic region where an sgRNA capable of binding to guide target sequence a will initiate a Cas9-induced double stranded DNA cleavage. The promoter insertion cassette is flanked by regions homologous to each side of the nuclease cut site to direct the cassette's insertion through the plant's endogenous homology directed repair mechanism. ii. a cassette for insertion of an intron between the promoter and the start codon of the gene. In this example, the insertion of the intron cassette is targeted to a genomic region where an sgRNA capable of binding to guide target sequence b will initiate a Cas9-induced double stranded DNA cleavage in a region near the 5′ UTR and the start codon of the transcription factor gene. The intron insertion cassette is flanked by regions homologous to each side of the nuclease cut site to direct the cassettes insertion through the plants endogenous homology directed repair mechanism. iii. a cassette for insertion of a promoter enhancer upstream of the endogenous promoter. In this example, the insertion of the enhancer cassette is targeted to a genomic region where an sgRNA capable of binding to guide target sequence c will initiate a Cas9-induced double stranded DNA cleavage. The enhancer insertion cassette is flanked by regions homologous to each side of the nuclease cut site to direct the cassette's insertion through the plant's endogenous homology directed repair mechanism. C. Illustration of the products of site-directed insertion for cassette i, ii, and/or iii into genomic DNA. While the illustration shows insertion of all three cassettes, one skilled in the art will understand that insertion(s) can be selected from one or more cassettes.

### DETAILED DESCRIPTION OF THE INVENTION

The following terms, unless otherwise indicated, shall be understood to have the following meanings:

As used herein we use the terms “crops” and “plants” interchangeably.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “recombinant expression construct”, which are used interchangeably, refers to any gene that is not a native gene, comprising regulatory and coding sequences that are not found together in nature. A “Cis-genic gene” is a chimeric gene where the DNA sequences making up the gene are from the same plant species or a sexually compatible plant species where the cis-genic gene is deployed in the same species from which the DNA sequences were obtained. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure. As used herein the term “coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. As used herein “gene” includes protein coding regions of the specific genes and the regulatory sequences both 5′ and 3′ which control the expression of the gene.

As used herein a “modified plant” refers to non-naturally occurring plants or crops engineered as described throughout herein.

As used herein a “control plant” means a plant that has not been modified as described in the present disclosure to impart an enhanced trait or altered phenotype. A control plant is used to identify and select a modified plant that has an enhanced trait or altered phenotype. For instance, a control plant can be a plant that has not been modified or has not been genome edited to express or to inhibit its endogenous gene product. A suitable control plant can be a non-transgenic plant of the parental line used to generate a transgenic plant, for example, a wild type plant devoid of a recombinant DNA. A suitable control plant can also be a transgenic plant that contains recombinant DNA that imparts other traits, for example, a transgenic plant having enhanced herbicide tolerance. A suitable control plant can in some cases be a progeny of a hemizygous transgenic plant line that does not contain the recombinant DNA, known as a negative segregant, or a negative isogenic line.

As used herein the terms “biomass yield” or “biomass content” refer to increase or decrease in the % dry weight in an amount greater than an otherwise identical plant, cultured under identical conditions, but lacking any corresponding modification, e.g., gene editing or the transgene in a control plant.

As used herein, the terms “increase activity”, “increase expression” or “upregulated” are used interchangeably and mean the activity of the transcription factor is increased or higher than the expression of the same gene in the same plant species before the gene was modified as described herein. The term also encompasses the situation where the activity of the transcription factor gene is upregulated in a tissue or at a stage of plant development as compared to the activity of the transcription factor gene in the tissue or developmental stage before the gene was modified. Upregulation should be understood to include an increase in the level or activity of a target gene in a cell and/or an increase in the expression of a particular target polypeptide in a cell which normally expresses the target polypeptide. For instance, a 1%, 5%, 10%, 20%, 30%, 40%, 50%, 60%, 70%, 80%, 90%, 100%, 2-fold, 5-fold, 10-fold, 20-fold, 50-fold, 100-fold increase in the level of activity of a target polypeptide in the cell. With respect to term “2-fold increase”, “upregulated 2-fold” and 100% increase is used interchangeably.

“Codon degeneracy” refers to divergence in the genetic code permitting variation of the nucleotide sequence without affecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for increased expression in a host cell, it is desirable to design the nucleic acid fragment such that its frequency of codon usage approaches the frequency of preferred codon usage of the host cell.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity). When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percent sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percent sequence identity” means the value determined by comparing two aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percent sequence identity.

The term “corn plant” includes whole plant, mature plants, seeds, shoots and seedlings, and parts, propagation material, plant organ tissue, protoplasts, callus and other cultures, for example cell cultures, derived from corn plants. The term “mature corn plants” refers to plants at any developmental stage beyond the seedling. The term “seedlings” refers to young, immature plants at an early developmental stage.

### PREFERRED EMBODIMENTS

The present disclosure relates to transcription factor genes in corn whose expression or activity can be modulated to increase corn crop performance and corn crops having increased expression of the transcription factor genes which have improved performance compared to the same corn plants with normal expression levels of these genes. Also disclosed are specific corn transcription factor gene sequences, DNA sequences, RNA sequences and materials and methods for modifying plant cells and plants such that they have increased expression of the transcription factor genes, methods for identifying corn plant cells and corn plants with increased expression of the transcription factor genes and methods for producing fertile corn plants with increased expression of the transcription factor genes wherein the modified corn plants have improved performance as compared to the same corn plants before they were modified to increase the expression of these genes.

In various aspects, the present invention provides corn transcription factors and genes encoding the corn transcription factors useful for practicing the disclosed invention and include those that can function as positive controllers in corn plants. Transcription factors function to either increase the activity of specific metabolic pathways or gene regulatory networks in plants or to decrease them. Herein we disclose corn transcription factors and genes encoding the corn transcription factors that function as positive controllers in corn and whose increased expression in corn is important for improved performance.

In one embodiment, the corn transcription factors comprise (a) one or more polypeptides comprising SEQ ID NOS: 87, 88, or 89. These sequences correspond to consensus sequences for three groups of corn transcription factors that function as positive controllers in corn and whose increased expression in corn is important for improved performance. In some examples, the corn transcription factors comprise (b) one or more polypeptides comprising SEQ ID NOS: 4, 6, 8, 10, 14, 16, 18, 20, 24, 26, 28, 30, 32, 41, 43 or 48. Also in some examples, the corn transcription factors comprise (c) one or more of the polypeptides set forth in (a) having at least 85%, 90%, 95% or higher sequence identity to one or more of the polypeptides set forth in (b). Also in some examples, the corn transcription factor genes comprise (d) one or more polynucleotides comprising SEQ ID NOS: 3, 5, 7, 9, 13, 15, 17, 19, 23, 25, 27, 29, 31, 40, 42 or 47. Also in some examples, the corn transcription factor genes comprise (e) one or more polynucleotides having at least 85%, 90%, 95% or higher sequence identity to one or more of the polynucleotides set forth in (d). Also, in some examples, the corn transcription factors comprise (f) one or more polypeptides encoded by one or more of the polynucleotides set forth in (d) or (e).

Thus, in one example the corn transcription factor comprises one or more of SEQ ID NOS: 4, 8, 10, 16, 18, 20, 26, 28, 30, 32, 41, 43, or 48.

In another example the corn transcription factor comprises one or more of SEQ ID NO: 6 (GRMZM2G110333), SEQ ID NO: 14 (GRMZM2G016434), and SEQ IO NO: 24 (GRMZM2G384528).

The present invention provides isolated nucleic acid molecules for genes encoding transcription factors, and variants thereof. Exemplary full-length nucleic acid sequences for genes encoding transcription factors and the corresponding amino acid sequences are presented in TABLE 1 and TABLE 2. The nucleic acid sequence can be preferably greater than 80%, 85%, 90%, 95%, 98%, 99%, 99.9% or even higher identity to the wild-type gene.

In another embodiment, the nucleic acid molecule of the present invention encodes a polypeptide having an amino acid sequence disclosed in TABLE 1 or TABLE 2. Preferably, the nucleic acid molecule of the present invention encodes a polypeptide sequence having at least 85%, 90% or 95% identity to the amino acid sequences shown in TABLE 1 or TABLE 2 and the identity can even more preferably be 96%, 97%, 98%, 99%, 99.9% or even higher.

According to another aspect of the present invention, isolated polypeptides (including muteins, allelic variants, fragments, derivatives, and analogs) encoded by the nucleic acid molecules of the present invention are provided. In one embodiment, the isolated polypeptide comprises the polypeptide sequence corresponding to a polypeptide sequence shown in TABLE 1 or TABLE 2.

In an alternative embodiment of the present invention, the isolated polypeptide comprises a polypeptide sequence at least 85%, 90%, 95% or higher sequence identity to a polypeptide sequence shown in TABLE 1 or TABLE 2. Preferably the isolated polypeptide of the present invention has at least 85%, 90%, 95%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or even higher identity to a polypeptide of SEQ ID NOS: 4, 6, 8, 10, 14, 16, 18, 20, 24, 26, 28, 30, 32, 41, 43 or 48.

The different families of transcription factors found in crops are described for example by Lin, et. al., (2014, BMC Genomics, 15, 818-820).

The modern corn genome contains around 39,000 thousand genes and about 2,500 of these are transcription factors (Lin, et. al., 2014, BMC Genomics, 15, 818-820). It is known that many plant species contain more than one copy of a specific gene and this invention encompasses all copies of the specific genes identified.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. BLASTP protein searches can be performed using default parameters. See, blast.ncbi.nlm.nih.gov/Blast.cgi.

Sequence alignments and percent similarity calculations may be determined using the Megalign program of the LASARGENE bioinformatics computing suite (DNASTAR Inc., Madison, Wis.) or using the AlignX program of the Vector NTI bioinformatics computing suite (Invitrogen, Carlsbad, Calif.). Multiple alignment of the sequences are performed using the Clustal method of alignment (Higgins and Sharp, CABIOS 5:151-153 (1989)) with the default parameters (GAP PENALTY=10, GAP LENGTH PENALTY=10). Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are GAP PENALTY=10, GAP LENGTH PENALTY=10, KTUPLE=2, GAP PENALTY=5, WINDOW=4 and DIAGONALS SAVED=4. A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Altschul, S. F. et al., J. Mol. Biol. 215:403-410 (1993)) and Gapped Blast (Altschul, S. F. et al., Nucleic Acids Res. 25:3389-3402 (1997)). BLASTN refers to a BLAST program that compares a nucleotide query sequence against a nucleotide sequence database.

Disclosed herein are corn (maize) transcription factor genes specified by SEQ ID NOS: 3, 5, 7, 9, 13, 15, 17, 19, 23, 25, 27, 29, 31, 40, 42 or 47, and methods for increasing their expression alone or in combinations in corn to improve corn performance are included in the scope of this invention.

Based on the disclosure herein, it will be apparent to a person of skill in the art how to use the genes and the proteins encoded by the genes identified by SEQ ID NOS: 3, 5, 7, 9, 13, 15, 17, 19, 23, 25, 27, 29, 31, 40, 42 or 47 by different methods to increase the expression of one or more of the transcription factor genes in corn such that the performance of the corn crop is improved.

In some embodiments, the polynucleotide is upregulated by using traditional genetic engineering methods which are well known in the art and have recently been reviewed by Qiudeng Que*, Sivamani Elumalai, Xianggan Li, Heng Zhong, Samson Nalapalli, Michael Schweiner, Xiaoyin Fei, Michael Nuccio, Timothy Kelliher, Weining Gu, Zhongying Chen, and Mary-Dell M. Chilton (2014) Frontiers in Plant Science 5, article 379, pp 1-19.

In some embodiments, the polynucleotide is upregulated by the use of new breeding techniques where targeted DNA sequence changes are facilitated thru the use of Zinc finger nuclease (ZFN) technology (ZFN-1, ZFN-2 and ZFN-3, see U.S. Pat. No. 9,145,565, incorporated by reference in its entirety), Oligonucleotide directed mutagenesis (ODM), Cisgenesis and intragenesis, RNA-dependent DNA methylation (RdDM, which does not necessarily change nucleotide sequence but can change the biological activity of the sequence), Grafting (on GM rootstock), Reverse breeding, Agro-infiltration (agro-infiltration “sensu stricto”, agro-inoculation, floral dip), Transcription Activator-Like Effector Nucleases (TALENs, see U.S. Pat. Nos. 8,586,363 and 9,181,535, incorporated by reference in their entireties), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641), engineered meganuclease re-engineered homing endonucleases, DNA guided genome editing (Gao et al., Nature Biotechnology (2016), doi: 10.1038/nbt.3547, incorporated by reference in its entirety), and synthetic genomics. A complete description of each of these techniques can be found in the report made by the Joint Research Center (JRC) Institute for Prospective Technological Studies of the European Commission in 2011 and titled “New plant breeding techniques—State-of-the-art and prospects for commercial development” website: ipts.jrc.ec.europa.eu/publications/pub.cfm?id=4100).

Modulation of candidate transcription factor genes are performed through known techniques in the art, such as without limitation, by genetic means, enzymatic techniques, chemicals methods, or combinations thereof. Activation may be conducted at the level of DNA, mRNA or protein, and inhibit the expression of one or more candidate transcription factor genes or the corresponding activity. Preferred activation methods affect the expression of the transcription factor gene and lead to the increase of gene product in the plant cells. Increased expression can be obtained via mutagenesis of the transcription factor gene. For example, a mutation in the coding sequence can induce, depending upon the nature of the mutation, increased activity of the protein; a mutation at or introduction of a splicing site can also increase expression and activity; a mutation in the promoter sequence can increase its activity and increase expression of the transcription factor gene. Mutagenesis can be performed, e.g., to modify the promoter, or by inserting an exogenous sequence, e.g., a transcription enhancer or intron, into said promoter. It can also be performed by inducing point mutations, e.g., using ethyl methanesulfonate (EMS) mutagenesis or radiation. The mutated alleles can be detected, e.g., by PCR, by using specific primers of the gene. Rodriguez-Leal et al. describe a promoter editing method that generates a pool of promoter variants that can be screened to evaluate their phenotypic impact (Rodriguez-Leal et al., 2017, Cell, 171, 1-11). This method can be incorporated into the present invention to upregulate native promoters of transcription factors of interest.

Various high-throughput mutagenesis and splicing methods are described in the prior art. By way of examples, we may cite “TILLING” (Targeting Induced Local Lesions In Genome)-type methods, described by Till, Comai and Henikoff (2007) (R. K. Varshney and R. Tuberosa (eds.), Genomics-Assisted Crop Improvement: Vol. 1: Genomics Approaches and Platforms, 333-349).

Corn plants comprising a mutation in the candidate transcription factor genes that increase the activity or stability of the protein product are also part of the goal of the present invention. This mutation can be, e.g., may be a point mutation of said coding sequence or of said promoter.

Enhanced expression of the transcription factor proteins can also be obtained by gene editing of the candidate genes. Examples of methods for editing genes in corn have recently been published (Svitashev, S., Young, J. K., Schwartz, C., Gao, H., Falco, S. C. and Cigan, A. M. 2015, Methods for targeted mutagenesis, precise gene editing, and site-specific gene insertion in maize using Cas9 and guide RNA. Plant Physiology 169, 931-945). Various methods can be used for gene editing, by using transcription activator-like effector nucleases (TALENs), clustered Regularly Interspaced Short Palindromic Repeats (CRISPR/Cas9) or zinc-finger nucleases (ZFN) techniques (as described in Belhaj et al, 2013, Plant Methods, vol 9, p 39, Chen et al, 2014 Methods Volume 69, Issue 1, p 2-8). Preferably, the enhancement of a transcription factor protein, or the enhancement of its expression, is obtained by using Clustered Regularly Interspaced Short Palindromic Repeats (CRISPR/Cas9) or CRISPR/Cpf1. The use of this technology in genome editing is well described in the art, for example in Fauser et al. (Fauser et al, 2014, The Plant Journal, Vol 79, p 348-359), and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). At least classes (Class I and II) and six types (Types I-VI) of Cas proteins have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences (protospacers), which direct Cas nucleases to the target site. The Type II CRISPR/Cas is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Cas9 is thus the hallmark protein of the Type II CRISPR-Cas system, and a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA.

Engineered systems utilize heterologous expression of Cas9 together with a single guide RNA (sgRNA), a synthetic fusion between a crRNA and part of the tracrRNA sequence, to introduce site-specific double strand breaks (DSBs) into genomic DNA of live cells from various organisms. For applications in eukaryotic organisms, codon optimized versions of Cas9, which is originally from the bacterium Streptococcus pyogenes, have been used. The sgRNA forms a complex with the Cas9 nuclease. The “guide” portion of the sgRNA (FIG. 6), which is about 20 nucleotides in length and located at the 5′ end of the sgRNA, is designed to be complementary to a DNA target sequence adjacent to a PAM sequence and confers DNA target specificity. Therefore, by modifying the sequence of the guide portion of the sgRNA, it is possible to create sgRNAs with different target specificities. The canonical length of the guide of the sgRNA is ˜20 bp. In plants, sgRNAs have been expressed using plant RNA polymerase III promoters, such as U6 and U3. Cas9 expression plasmids for use in the methods of the invention can be constructed as described in the art.

The increased expression in modified engineered plants or plant cells can be verified based on the phenotypic characteristics of their offspring; homozygous plants or plant cells for a mutation increasing the expression of the transcription factor gene have a content of gene product that is higher than that of the wild plants (not carrying the mutation in the gene) from which they originated. Alternatively, a desirable phenotypic characteristic such as photosynthesis rate, photosynthetic electron transport rate, biomass yield, seed yield, or seed oil content is measured and is at least 10% higher, preferably at least 20% higher, at least preferably 30% higher, preferably at least 40% higher, preferably at least 50% higher than that of the control plants from which they originated. Photosynthetic parameters, such as photochemical quantum yield (Y), non-photochemical quenching (NPQ), and electron transport rate (ETR) can be measured in plants using commercially available machines, such as the Dual-PAM-100 Measuring System (Heinz Walz Gmbh, Effeltrich, Germany). Increases in Y in plants represent increases in the portion of absorbed quanta that is converted into chemically fixed energy by the photosystem I (PSI) and photosystem II (PSII) reaction centers. The photosynthetic electron transport rates are often referred to as the electron transport rates of PSI and PSII. Increases in the electron transport rate of PSII (indicative of the rate of non-cyclic electron transfer), and the electron transport rate of PSI (indicative of both cyclic and non-cyclic electron transfer), can be determined. NPQ is a mechanism that plants use to protect themselves from high light intensity and manipulation of NPQ can increase yield (Hubbart et al., 2018, Nature Communications Biology, 1, Article 22).

More preferably, seed yield is at least 5%, at least 10%, at least 20%, at least 40%, at least 60% higher, at least 70% higher, at least 80% higher, at least 90% higher than that of the control plants from which they originated. More preferably, seed yield or seed oil content is at least 100% higher, at least 150% higher, at least 200% higher than that of the control plants from which they originated.

The expression of the target gene or genes in the crops of interest can be increased by any method known in the art, including the transgene based expression of the gene or through genome editing or mutagenesis to modify the DNA sequence of the promoter sequences of the genes disclosed herein directly in the plant cell chromosome.

Genome editing is a preferred method for practicing this invention. As used herein the terms “genome editing,” “genome edited”, and “genome modified” are used interchangeably to describe plants with specific DNA sequence changes in their genomes wherein those DNA sequence changes include changes of specific nucleotides, the deletion of specific nucleotide sequences or the insertion of specific nucleotide sequences.

As used herein “method for genome editing” includes all methods for genome editing technologies to precisely remove genes, gene fragments, or to insert new DNA sequences into genes, to alter the DNA sequence of control sequences or protein coding regions to reduce or increase the expression of target genes in plant genomes (Belhaj, K. 2013, Plant Methods, 9, 39; Khandagale & Nadal, 2016, Plant Biotechnol Rep, 10, 327). Preferred methods involve the in vivo site-specific cleavage to achieve double stranded breaks in the genomic DNA of the plant genome at a specific DNA sequence using nuclease enzymes and the host plant DNA repair system. There are multiple methods to achieve double stranded breaks in genomic DNA, and thus achieve genome editing, including the use of zinc finger nucleases (ZFN), transcription activator-like effector nucleases (TALENs), engineered meganucleases, and the CRISPR/Cas system (CRISPR is an acronym for clustered, regularly interspaced, short, palindromic repeats and Cas an abbreviation for CRISPR-associated protein) (for review see Khandagal & Nadal, Plant Biotechnol Rep, 2016, 10, 327). US Patent Application 2016/0032297 to Dupont describes these methods in detail. In some cases, the sequence specificity for the target gene in the plant genome is dependent on engineering specific nucleases like zinc finger nucleases (ZFN), which include an engineered DNA-binding zinc finger domain linked to a non-specific endonuclease domain such as FokI, or Tal effector nuclease (TALENS) to recognize the target DNA sequence in the plant genome. The CRISPR/Cas genome editing system is a preferred method because of its sequence targeting flexibility. This technology requires a source of the Cas enzyme and a sgRNA containing a short guide (˜20 bp), with sequence complementarity to the target DNA sequence in the plant genome. Depending on the type of Cas enzyme, alternatively a DNA, an RNA/DNA hybrid, or a double stranded DNA guide polynucleotide can be used. The guide portion of this guide polynucleotide directs the Cas enzyme to the desired cut site for cleavage with a recognition sequence for binding the Cas enzyme. As used herein the term Cas nuclease includes any nuclease which site-specifically recognizes CRISPR sequences based on guide RNA or DNA sequences and includes Cas9, Cpf1 and others described below. CRISPR/Cas genome editing, is a preferred way to edit the genomes of complex organisms (Sander & Joung, 2013, Nat Biotech, 2014, 32, 347; Wright et al., 2016, Cell, 164, 29) including plants (Zhang et al., 2016, Journal of Genetics and Genomics, 43, 151; Puchta, H., 2016, Plant J., 87, 5; Khandagale & Nadaf, 2016, PLANT BIOTECHNOL REP, 10, 327). US Patent Application 2016/020822 to Dupont has an extensive description of the materials and methods useful for genome editing in plants using the CRISPR/Cas9 system and describes many of the uses of the CRISPR/Cas9 system for genome editing of a range of gene targets in crops.

There are many variations of the CRISPR/Cas system that can be used for this technology including the use of wild-type Cas9 from Streptococcus pyogenes (Type II Cas) (Barakate & Stephens, 2016, Frontiers in Plant Science, 7, 765; Bortesi & Fischer, 2015, Biotechnology Advances 5, 33, 41; Cong et al., 2013, Science, 339, 819; Rani et al., 2016, Biotechnology Letters, 1-16; Tsai et al., 2015, Nature biotechnology, 33, 187), the use of a Tru-gRNA/Cas9 in which off-target mutations were significantly decreased (Fu et al., 2014, Nature biotechnology, 32, 279; Osakabe et al., 2016, Scientific Reports, 6, 26685; Smith et al., 2016, Genome biology, 17, 1; Zhang et al., 2016, Scientific Reports, 6, 28566), a high specificity Cas9 (mutated S. pyogenes Cas9) with little to no off target activity (Kleinstiver et al., 2016, Nature 529, 490; Slaymaker et al., 2016, Science, 351, 84), the Type I and Type III Cas Systems in which multiple Cas proteins need to be expressed to achieve editing (Li et al., 2016, Nucleic acids research, 44:e34; Luo et al., 2015, Nucleic acids research, 43, 674), the Type V Cas system using the Cpf1 enzyme (Kim et al., 2016, Nature biotechnology, 34, 863; Toth et al., 2016, Biology Direct, 11, 46; Zetsche et al., 2015, Cell, 163, 759), DNA-guided editing using the NgAgo Argonaute enzyme from Natronobacterium gregoryi that employs guide DNA (Xu et al., 2016, Genome Biology, 17, 186), and the use of a two vector system in which Cas9 and sgRNA expression cassettes are carried on separate vectors (Cong et al., 2013, Science, 339, 819). A unique nuclease Cpf1, an alternative to Cas9 has advantages over the Cas9 system in reducing off-target edits which creates unwanted mutations in the host genome. Examples of crop genome editing using the CRISPR/Cpf1 system include rice (Tang et. al., 2017, Nature Plants 3, 1-5; Wu et. al., 2017, Molecular Plant, Mar. 16, 2017) and soybean (Kim et., al., 2017, Nat Commun. 8, 14406).

Methods for constructing the genome modified corn plant cells and corn plants include introducing into plant cells a vector comprising a gene expression construct of one or more of the corn transcription factor genes and a second gene expression construct comprising a selectable marker gene.

Methods for constructing the genome modified plant cells and plants include introducing into plant cells a site-specific nuclease to cleave the plant genome at the target site or target sites and the guide sequences. Modification to the DNA sequence at the cleavage site then occurs through the plant cell's natural DNA repair processes. In a preferred case using the CRISPR system the target site in the plant genome is determined by providing single guide RNA (sgRNA) sequences.

A “guide polynucleotide” also relates to a polynucleotide sequence that can form a complex with a Cas endonuclease and enables the Cas endonuclease to recognize and optionally cleave a DNA target site. The guide polynucleotide can be a single molecule (i.e. a single guide RNA (sgRNA) that is a synthetic fusion between a crRNA and part of the tracrRNA sequence) or a two molecules (i.e. the crRNA and tracrRNA as found in natural Cas9 systems in bacteria). The guide polynucleotide sequence can be provided as an RNA sequence or can be transcribed from a DNA sequence to produce an RNA sequence. The guide polynucleotide sequence can also be provided as a combination RNA-DNA sequence (see for example, Yin, H. et al., 2018, Nature Chemical Biology, 14, 311).

As used herein “guide RNA” sequences comprise a variable targeting domain, called the “guide”, complementary to the target site in the genome, and an RNA sequence that interacts with the Cas9 or Cpf1 endonuclease, called the “guide RNA scaffold”. A guide polynucleotide that solely comprises ribonucleic acids is also referred to as a “guide RNA”.

As used herein the “guide target sequence” refers to the sequence of the genomic DNA adjacent to a PAM site, where the sgRNA will bind to cleave the DNA. The “guide target sequence” is often complementary to the “guide” portion of the sgRNA, however several mismatches, depending on their position, can be tolerated and still allow Cas mediated cleavage of the DNA.

The method also provides introducing single guide RNAs (sgRNAs) into plants. The single guide RNAs (sgRNAs) include nucleotide sequences that are complementary to the target chromosomal DNA. The sgRNAs can be, for example, engineered single chain guide RNAs that comprise a crRNA sequence (complementary to the target DNA sequence) and a common tracrRNA sequence, or as crRNA-tracrRNA hybrids. The sgRNAs can be introduced into the cell or the organism as a DNA with an appropriate promoter, as an in vitro transcribed RNA, or as a synthesized RNA. Basic guidelines for designing the guide RNAs for any target gene of interest are well known in the art as described for example by Brazelton et al. (Brazelton, V. A. et al., 2015, GM Crops & Food, 6, 266-276) and Zhu (Zhu, L. J. 2015, Frontiers in Biology, 10, 289-296).

### Target Sequence for Increasing Expression

Examples of mutations that may lead to increased activity of the transcription factor protein are mutations to the coding sequence that give rise to amino acid changes in the encoded protein.

In certain preferred embodiments, the guide polynucleotide/Cas endonuclease system can be used to allow for the insertion of a promoter or promoter element of any one the transcription factor sequences of the invention, wherein the promoter insertion (or promoter element deletion) results in any one of the following or any one combination of the following: a permanently activated gene locus, an increased promoter activity (increased promoter strength), an increased promoter tissue specificity, a decreased promoter tissue specificity, a new promoter activity, an extended window of gene expression, a modification of the timing or developmental progress of gene expression, a mutation of DNA binding elements and/or an addition of DNA binding elements. Promoter elements to be deleted can be, but are not limited to, promoter core elements, promoter enhancer elements or 35 S enhancer elements (CaMV35S enhancers (Benfey et al, EMBO J, August 1989; 8(8): 2195-2202)). The promoter or promoter fragment to be deleted can be endogenous, artificial, pre-existing, or transgenic to the cell that is being edited. Preferably the promoter element is endogenous to the cell that is being edited

In yet another embodiment, the genomic sequence of interest to be modified is an intron site of any one of the transcription factor sequences of the invention, wherein the modification consists of inserting an intron enhancing motif into the intron which results in modulation of the transcriptional activity of the gene comprising said intron.

In a further embodiment, methods provide for modifying alternative splicing sites of any one of the transcription factor sequences of the invention resulting in enhanced production of the functional gene transcripts and gene products (proteins).

In additional embodiments, the modification of the transcription factor sequences of the invention include editing the intron borders of alternatively spliced genes to alter the accumulation of splice variants.

In other embodiments, the guide polynucleotide/Cas endonuclease system can be used to modify or replace a coding sequence of the transcription factor in the genome of a plant cell, wherein the modification or replacement results in any one of the following, or any one combination of the following: an increased protein activity, an increased protein functionality, a site specific mutation, a protein domain swap, a protein knock-out, a new protein functionality, a modified protein functionality.

The guide RNA/Cas endonuclease system can be used to allow for the insertion of a promoter element to increase the expression of the transcription factor sequences of the invention. Promoter elements, such as enhancer elements, are often introduced in promoters driving gene expression cassettes in multiple copies for trait gene testing or to produce transgenic plants expressing specific traits. Enhancer elements can be, but are not limited to, a 35S enhancer element (Benfey et al, EMBO J, August 1989; 8(8): 2195-2202). In some plants (events), the enhancer elements can cause a desirable phenotype, a yield increase, or a change in expression pattern of the trait of interest that is desired. It may be desired to remove the extra copies of the enhancer element while keeping the trait gene cassettes intact at their integrated genomic location. The guide RNA/Cas endonuclease can be used to remove the unwanted enhancing element from the plant genome. A guide RNA can be designed to contain a variable targeting region targeting a target site sequence of 12-30 bps adjacent to a NGG (PAM) in the enhancer. The Cas endonuclease can make cleavage to insert one or multiple enhancers. The guide RNA/Cas endonuclease system can be introduced by either Agrobacterium or particle gun bombardment. Alternatively, nanotube or nanoparticle mediated DNA delivery (Kwak et al., 2019, Nature Nanotechnology, DOT 10.1038/s41565-019-0375-4) (Demirer et al, 2019, Nature Nanotechnology, DOI 10.1038/s41565-019-0382-5) can be used. Two different guide RNAs (targeting two different genomic target sites) can be used to remove multiple enhancer elements from the genome of a plant.

In some embodiments, the genome modified plant has improved performance as compared to a plant of the same type which does not have the genome modification. The improved performance of the genome modified plant includes for example, higher photosynthesis rates, higher photosynthetic electron transport rate, higher non-photochemical quenching, reduced photorespiration rates, higher biomass yield or content, higher seed yield, improved harvest index, higher seed oil content, improved nutritional composition, improved nitrogen use efficiency, drought resistance, flood resistance, disease resistance, salt tolerance, higher CO₂ assimilation rate, or lower transpiration rate. The genome modified plant can have a CO₂ assimilation rate that is higher than for a corresponding reference plant not comprising the genome modification. For example, the genome modified plant can have a CO₂ assimilation rate that is at least 5% higher, at least 10% higher, at least 20% higher, at least 40% higher, at least 60% higher, at least 100% higher, at least 200% higher or at least 400% higher than for a corresponding reference plant not comprising the genome modification.

The genome modified plant can also have a transpiration rate that is lower than for a corresponding reference plant not comprising the genome modification. For example, the genome modified plant can have a transpiration rate that is at least 5% lower, at least 10% lower, at least 20% lower, at least 40% lower, at least 60% lower or at least 100% lower than for a corresponding reference plant not comprising the genome modification.

The genome modified plant can have a seed yield or a seed oil content that is higher than for a corresponding reference plant not comprising the genome modification. For example, the genome modified plant can have a seed yield or seed oil content that is at least 5% higher, at least 10% higher, at least 20% higher, at least 40% higher, at least 60% higher, at least 80% higher or at least 100% higher, than for a corresponding reference plant not comprising the genome modification.

The genome modified plant can have a seed yield that is higher than for a corresponding reference plant not comprising the genome modification. For example, the genome modified plant can have a seed yield that is at least 5% higher, at least 10% higher, at least 20% higher, at least 40% higher, at least 60% higher, at least 80% higher or at least 100% higher, than for a corresponding reference plant not comprising the genome modification.

### Plants of Interest

Transcription factor genes, including specific corn transcription factor gene sequences are useful as targets for upregulation, alone or in combinations, to improve corn crop performance are described herein. Preferably the transcription factor genes are upregulated in an inbred corn line to reduce the time for development and testing of the impact of the upregulated transcription factor in corn hybrids. Methods of upregulating the transcription factor genes in corn include transgenic approaches and the use of site-specific nucleases, guide RNAs, guide RNA-DNA hybrids and guide DNAs. DNA constructs useful in the methods are described herein. Methods for introducing either the genetic construct or the site-specific nuclease and guide RNAs into plant cells and plant tissues are also described herein and methods for identifying plant cells, plant tissue and fertile plants having increased expression of the transcription factor genes made using these methods are disclosed herein. As used herein, “transgenic” refers to an organism in which a nucleic acid fragment containing a heterologous or “non-native” nucleotide sequence has been introduced. Preferably the non-native nucleotide sequence is derived from nucleotide sequences naturally present in corn. The increased expression of the transcription factors introduced into the plants are stable, inheritable and impart improved plant performance.

### Modified Plant Genomes Using CRISPR/Cas, Guide RNAs

Examples of simultaneous CRISPR/Cas9 or CRISPR/Cpf1 gene editing at multiple target sites, or multiplex genome editing, have been described for both mammalian cells and plants, and can be achieved by expressing one or more sgRNAs to target multiple genome sites within the organism. This has been demonstrated in rice with the use of seven sgRNAs for editing (Ma et al., 2015, Mol Plant, 8, 1274). It is therefore an objective of this invention to use multiple sgRNAs to direct the insertion of a specific DNA sequence to multiple sites in the plant genome using one or more of the previous embodiments of the invention.

### Methods for DNA Insertion at the Target Site

The methods for achieving the genome modification are described using the CRISPR/Cas9 system although it will be appreciated that other variations of the CRISPR/Cas systems can also be used including one that uses guide DNA sequences. The method requires the introduction of the site-specific nuclease and guide RNA into the nucleus of plant cells from the target crop. These may vary for different crop species or due to preference or skill set of the crop scientists.

One skilled in the art can produce and introduce proteins or DNA into many crop types using plant cell protoplasts. Preferably the plant protoplasts once genome edited can be regenerated into stable fertile plants suitable for crop breeding programs. For example, protoplast transformation and hence genome editing is useful for modifying the genomes of Camelina, canola, soybean, corn, rice, wheat, potato, alfalfa, tomato, cotton, barley and many other crops of interest. The Cas9 nuclease enzyme can be combined with the sgRNAs to form protein/RNA particles which can then be introduced into the plant protoplasts.

Methods for Identifying or Selecting Plant Cells with the Targeted Genome Edits Methods of Plant Transformation

Known transformations methods can be used upregulate one or more gene sequences of the invention.

### Vectors

Several plant transformation vector options are available, including those described in Gene Transfer to Plants, 1995, Potrykus et al., eds., Springer-Verlag Berlin Heidelberg New York, Transgenic Plants: A Production System for Industrial and Pharmaceutical Proteins, 1996, Owen et al., eds., John Wiley & Sons Ltd. Eng, and Methods in Plant Molecular Biology: A Laboratory Course Manual, 1995, Maliga et al., eds., Cold Spring Laboratory Press, New York). Plant transformation vectors generally include one or more coding sequences of interest under the transcriptional control of 5′ and 3′ regulatory sequences, including a promoter, a transcription termination and/or polyadenylation signal, and a selectable or screenable marker gene.

Many vectors are available for transformation using Agrobacterium tumefaciens. These typically carry at least one T-DNA sequence and include vectors such as pBIN19. Typical vectors suitable for Agrobacterium transformation include the binary vectors pCIB200 and pCIB2001, as well as the binary vector pCIB 10 and hygromycin selection derivatives thereof (See, for example, U.S. Pat. No. 5,639,949).

Transformation without the use of Agrobacterium tumefaciens circumvents the requirement for T-DNA sequences in the chosen transformation vector, and consequently vectors lacking these sequences are utilized in addition to vectors such as the ones described above which contain T-DNA sequences. The choice of vector for transformation techniques that do not rely on Agrobacterium depends largely on the preferred selection for the species being transformed. Typical vectors suitable for non-Agrobacterium transformation include pCIB3064, pSOG 19, and pSOG35 (See, for example, U.S. Pat. No. 5,639,949). Alternatively, DNA fragments containing the transgene and the necessary regulatory elements for expression of the transgene can be excised from a plasmid and delivered to the plant cell using microprojectile bombardment-mediated methods.

### Protocols

Transformation protocols as well as protocols for introducing nucleotide sequences into plants may vary depending on the type of plant or plant cell targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (Townsend et al., U.S. Pat. No. 5,563,055; Zhao et al. WO US98/01268), direct gene transfer (Paszkowski et al. (1984) EMBO J. 3:2717-2722), and ballistic particle acceleration (see, for example, Sanford et al., U.S. Pat. No. 4,945,050; Tomes et al. (1995) Plant Cell, Tissue, and Organ Culture: Fundamental Methods, ed. Gamborg and Phillips (Springer-Verlag, Berlin); and McCabe et al. Biotechnology 6:923-926 (1988)). Also see Weissinger et al. Ann. Rev. Genet. 22:421-477 (1988); Sanford et al. Particulate Science and Technology 5:27-37 (1987) (onion); Klein et al. Proc. Natl. Acad. Sci. USA 85:4305-4309 (1988) (maize); Klein et al. Biotechnology 6:559-563 (1988) (maize); Tomes, U.S. Pat. No. 5,240,855; Buising et al., U.S. Pat. Nos. 5,322,783 and 5,324,646; Tomes et al. (1995) in Plant Cell, Tissue, and Organ Culture: Fundamental Methods, ed. Gamborg (Springer-Verlag, Berlin) (maize); Klein et al. Plant Physiol. 91:440-444 (1988) (maize); Fromm et al. Biotechnology 8:833-839 (1990) (maize); Hooykaas-Van Slogteren et al. Nature 311:763-764 (1984); Bowen et al., U.S. Pat. No. 5,736,369 (cereals); Bytebier et al. Proc. Natl. Acad. Sci. USA 84:5345-5349 (1987) (Liliaceae); De Wet et al. in The Experimental Manipulation of Ovule Tissues, ed. Chapman et al. (Longman, N.Y.), pp. 197-209 (1985) (pollen); Kaeppler et al. Plant Cell Reports 9:415-418 (1990) and Kaeppler et al. Theor. Appl. Genet. 84:560-566 (1992) (whisker-mediated transformation); D'Halluin et al. Plant Cell 4:1495-1505 (1992) (electroporation); Li et al. Plant Cell Reports 12:250-255 (1993) and Christou and Ford Annals of Botany 75:407-413 (1995) (rice); Osjoda et al. Nature Biotechnology 14:745-750 (1996) (maize via Agrobacterium tumefaciens). References for protoplast transformation and/or gene gun (also known as biolistics) are described in WO 2010/037209. Methods for transforming plant protoplasts are available including transformation using polyethylene glycol (PEG), electroporation, and calcium phosphate precipitation (see for example Potrykus et al., 1985, Mol. Gen. Genet., 199, 183-188; Potrykus et al., 1985, Plant Molecular Biology Reporter, 3, 117-128). Methods for plant regeneration from protoplasts have also been described [Evans et al., in Handbook of Plant Cell Culture, Vol 1, (Macmillan Publishing Co., New York, 1983); Vasil, IK in Cell Culture and Somatic Cell Genetics (Academic, Oro, 1984)].

Transformation protocols as well as protocols for introducing nucleotide sequences into plants may vary depending on the type of plant or plant cell, i.e., monocot or dicot, targeted for transformation.

The transformed cells are grown into plants in accordance with conventional techniques. See, for example, McCormick et al., 1986, Plant Cell Rep. 5: 81-84. These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

In planta methods have also been used for transformation of germ cells in maize (pollen, Wang et al. 2001, Acta Botanica Sin., 43, 275-279; Zhang et al., 2005, Euphytica, 144, 11-22; pistils, Chumakov et al. 2006, Russian J. Genetics, 42, 893-897; Mamontova et al. 2010, Russian J. Genetics, 46, 501-504) and Sorghum (pollen, Wang et al. 2007, Biotechnol. Appl. Biochem., 48, 79-83).

### Selection

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the DNA construct for introducing the targeted insertion of the DNA sequence elements producing the desired level of desired polypeptide(s) in the desired tissue and cellular location.

The cells that have been transformed may be grown into plants in accordance with conventional techniques. See, for example, McCormick et al. Plant Cell Reports 5:81-84(1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Transgenic plants can be produced using conventional techniques to express any genes of interest in plants or plant cells (Methods in Molecular Biology, 2005, vol. 286, Transgenic Plants: Methods and Protocols, Pena L., ed., Humana Press, Inc. Totowa, N.J.; Shyamkumar Barampuram and Zhanyuan J. Zhang, Recent Advances in Plant Transformation, in James A. Birchler (ed.), Plant Chromosome Engineering: Methods and Protocols, Methods in Molecular Biology, vol. 701, Springer Science+Business Media). Typically, gene transfer, or transformation, is carried out using explants capable of regeneration to produce complete, fertile plants. Generally, a DNA or an RNA molecule to be introduced into the organism is part of a transformation vector. A large number of such vector systems known in the art may be used, such as plasmids. The components of the expression system can be modified, e.g., to increase expression of the introduced nucleic acids. For example, truncated sequences, nucleotide substitutions or other modifications may be employed. Expression systems known in the art may be used to transform virtually any plant cell under suitable conditions. A transgene comprising a DNA molecule encoding a gene of interest is preferably stably transformed and integrated into the genome of the host cells. Transformed cells are preferably regenerated into whole fertile plants. Detailed descriptions of transformation techniques are within the knowledge of those skilled in the art.

Plant promoters can be selected to control the expression of the transgene in different plant tissues or organelles for all of which methods are known to those skilled in the art (Gasser & Fraley, 1989, Science 244: 1293-1299). In one embodiment, promoters are selected from those of eukaryotic or synthetic origin that are known to yield high levels of expression in plants and algae. In a preferred embodiment, promoters are selected from those that are known to provide high levels of expression in monocots.

Constitutive promoters include, for example, the core promoter of the Rsyn7 promoter and other constitutive promoters disclosed in WO 99/43838 and U.S. Pat. No. 6,072,050, the core CaMV 35S promoter (Odell et al., 1985, Nature 313: 810-812), rice actin (McElroy et al., 1990, Plant Cell 2: 163-171), ubiquitin (Christensen et al., 1989, Plant Mol. Biol. 12: 619-632; Christensen et al., 1992, Plant Mol. Biol. 18: 675-689), pEMU (Last et al., 1991, Theor. Appl. Genet. 81: 581-588), MAS (Velten et al., 1984, EMBO J. 3: 2723-2730), and ALS promoter (U.S. Pat. No. 5,659,026). Other constitutive promoters are described in U.S. Pat. Nos. 5,608,149; 5,608,144; 5,604,121; 5,569,597; 5,466,785; 5,399,680; 5,268,463; and 5,608,142.

“Tissue-preferred” promoters can be used to target gene expression within a particular tissue. Compared to chemically inducible systems, developmentally and spatially regulated stimuli are less dependent on penetration of external factors into plant cells. Tissue-preferred promoters include those described by Van Ex et al., 2009, Plant Cell Rep. 28: 1509-1520; Yamamoto et al., 1997, Plant J. 12: 255-265; Kawamata et al., 1997, Plant Cell Physiol. 38: 792-803; Hansen et al., 1997, Mol. Gen. Genet. 254: 337-343; Russell et al., 199), Transgenic Res. 6: 157-168; Rinehart et al., 1996, Plant Physiol. 112: 1331-1341; Van Camp et al., 1996, Plant Physiol. 112: 525-535; Canevascini et al., 1996, Plant Physiol. 112: 513-524; Yamamoto et al., 1994, Plant Cell Physiol. 35: 773-778; Lam, 1994, Results Probl. Cell Differ. 20: 181-196, Orozco et al., 1993, Plant Mol. Biol. 23: 1129-1138; Matsuoka et al., 1993, Proc. Natl. Acad. Sci. USA 90: 9586-9590, and Guevara-Garcia et al., 1993, Plant J. 4: 495-505. Such promoters can be modified, if necessary, for weak expression.

Any of the described promoters can be used to control the expression of one or more of the genes of the invention, their homologs and/or orthologs as well as any other genes of interest in a defined spatiotemporal manner.

### Expression Cassettes

Nucleic acid sequences intended for expression in transgenic plants are first assembled in expression cassettes behind a suitable promoter active in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be transferred to the plant transformation vectors described infra. The following is a description of various components of typical expression cassettes.

A variety of transcriptional terminators are available for use in expression cassettes. These are responsible for the termination of transcription beyond the transgene and the correct polyadenylation of the transcripts. Appropriate transcriptional terminators are those that are known to function in plants and include the CaMV 35S terminator, the tm1 terminator, the nopaline synthase terminator and the pea rbcS E9 terminator. These are used in both monocotyledonous and dicotyledonous plants.

Individual plants within a population of transgenic plants that express a recombinant gene(s) may have different levels of gene expression. The variable gene expression is due to multiple factors including multiple copies of the recombinant gene, chromatin effects, and gene suppression. Accordingly, a phenotype of the transgenic plant may be measured as a percentage of individual plants within a population. The yield of a plant can be measured simply by weighing. The yield of seed from a plant can also be determined by weighing. The increase in seed weight from a plant can be due to a number of factors, an increase in the number or size of the seed pods, an increase in the number of seed or an increase in the number of seed per plant. In the laboratory or greenhouse seed yield is usually reported as the weight of seed produced per plant and in a commercial crop production setting yield is usually expressed as weight per acre or weight per hectare.

A recombinant DNA construct including a plant-expressible gene or other DNA of interest is inserted into the genome of a plant by a suitable method. Suitable methods include, for example, Agrobacterium tumefaciens-mediated DNA transfer, direct DNA transfer, liposome-mediated DNA transfer, electroporation, co-cultivation, diffusion, particle bombardment, microinjection, gene gun, calcium phosphate coprecipitation, viral vectors, nanotube or nanoparticle mediated delivery, and other techniques. Suitable plant transformation vectors include those derived from a Ti plasmid of Agrobacterium tumefaciens. In addition to plant transformation vectors derived from the Ti or root-inducing (Ri) plasmids of Agrobacterium, alternative methods can be used to insert DNA constructs into plant cells. A transgenic plant can be produced by selection of transformed seeds or by selection of transformed plant cells and subsequent regeneration.

In one embodiment, the transgenic plants are grown (e.g., on soil) and harvested. In one embodiment, above ground tissue is harvested separately from below ground tissue. Suitable above ground tissues include shoots, stems, leaves, flowers, grain, and seed. Exemplary below ground tissues include roots and root hairs. In one embodiment, whole plants are harvested and the above ground tissue is subsequently separated from the below ground tissue.

Genetic constructs may encode a selectable marker to enable selection of transformation events. There are many methods that have been described for the selection of transformed plants [for review see (Miki et al., Journal of Biotechnology, 2004, 107, 193-232) and references incorporated within]. Selectable marker genes that have been used extensively in plants include the neomycin phosphotransferase gene nptII (U.S. Pat. Nos. 5,034,322, 5,530,196), hygromycin resistance gene (U.S. Pat. No. 5,668,298, Waldron et al., (1985), Plant Mol Biol, 5:103-108; Zhijian et al., (1995), Plant Sci, 108:219-227), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175; 7,045,684). Other suitable selectable markers include, but are not limited to, genes encoding resistance to chloramphenicol (Herrera Estrella et al., (1983), EMBO J, 2:987-992), methotrexate (Herrera Estrella et al., (1983), Nature, 303:209-213; Meijer et al, (1991), Plant Mol Biol, 16:807-820); streptomycin (Jones et al., (1987), Mol Gen Genet, 210:86-91); bleomycin (Hille et al., (1990), Plant Mol Biol, 7:171-176); sulfonamide (Guerineau et al., (1990), Plant Mol Biol, 15:127-136); bromoxynil (Stalker et al., (1988), Science, 242:419-423); glyphosate (Shaw et al., (1986), Science, 233:478-481); phosphinothricin (DeBlock et al., (1987), EMBO J, 6:2513-2518).

Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson et al., Nat Biotechnol, 2004, 22, 455-8). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of transgenic plants.

Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO J. 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein.

Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (Tzfira et al. (2005), Plant Molecular Biology 57: 503-516) and Verkhusha and Lukyanov (Verkhusha, V. V. and K. A. Lukyanov (2004), Nat Biotech 22: 289-296) whose references are incorporated in entirety. Improved versions of many of the fluorescent proteins have been made for various applications. Based on the disclosure herein, it will be apparent to a person of skill in the art how to use of the improved versions of these proteins or combinations of these proteins for selection of transformants.

The plants modified for enhanced performance by increasing the expression of the transcription factor genes or transcription factor gene combinations may be combined or stacked with input traits by crossing or plant breeding. Useful input traits include herbicide resistance and insect tolerance, for example a plant that is tolerant to the herbicide glyphosate and that produces the Bacillus thuringiensis (BT) toxin. Glyphosate is a herbicide that prevents the production of aromatic amino acids in plants by inhibiting the enzyme 5-enolpyruvylshikimate-3-phosphate synthase (EPSP synthase). The overexpression of EPSP synthase in a crop of interest allows the application of glyphosate as a weed killer without killing the modified plant (Suh, et al., J. M Plant Mol. Biol. 1993, 22, 195-205). BT toxin is a protein that is lethal to many insects providing the plant that produces it protection against pests (Barton, et al. Plant Physiol. 1987, 85, 1103-1109). Other useful herbicide tolerance traits include but are not limited to tolerance to Dicamba by expression of the dicamba monoxygenase gene (Behrens et al, 2007, Science, 316, 1185), tolerance to 2,4-D and 2,4-D choline by expression of a bacterial aad-1 gene that encodes for an aryloxyalkanoate dioxygenase enzyme (Wright et al., Proceedings of the National Academy of Sciences, 2010, 107, 20240), glufosinate tolerance by expression of the bialophos resistance gene (bar) or the pat gene encoding the enzyme phosphinotricin acetyl transferase (Droge et al., Planta, 1992, 187, 142), as well as genes encoding a modified 4-hydroxyphenylpyruvate dioxygenase (HPPD) that provides tolerance to the herbicides mesotrione, isoxaflutole, and tembotrione (Siehl et al., Plant Physiol, 2014, 166, 1162). The plants modified for enhanced yield by reducing the expression of the transcription factor genes or transcription factor gene combinations may be combined or stacked with other genes which improve plant performance.

Unless otherwise defined, all technical and scientific terms used herein have the same meaning as commonly understood by one of ordinary skill in the art to which this present invention pertains. Exemplary methods and materials are described below, although methods and materials similar or equivalent to those described herein can also be used in the practice of the present invention and will be apparent to those of skill in the art.

All patents, publications and other references mentioned herein are incorporated by reference in their entirety. In case of conflict, the present specification, including definitions, will control. The materials, methods, and examples are illustrative only and not intended to be limiting.

### EXAMPLES

### Example 1. Identification of Maize Orthologs to Switchgrass Transcription Factors

Over expression of the switchgrass transcription factors STR1 (SEQ ID NOS: 1 and 2), STIF1 (SEQ ID NOS: 11 and 12), and BMY1 (SEQ ID NOS: 21 and 22) in switchgrass have been previously been shown to increase biomass yield, photosynthetic parameters, and the content of photosynthetic pigments, soluble sugars, and starch and a maize sequence based ortholog for each switchgrass gene has been identified (WO2014100289). The switchgrass transcription factors were originally identified from a rice transcriptome regulatory association network (WO2014100289). Improvements to whole-genome datasets has more recently allowed the identification of additional orthologs to STR1, STIF1, and BMY1 in maize (TABLE 1 and TABLE 2) as well as homologs to STR1, STIF1, and BMY1 in switchgrass (TABLE 2). Manipulation of expression of these genes, through transgenic or genome editing approaches, can be used to increase yield in maize.

To identify additional maize transcription factor genes, the switchgrass STR1 (SEQ ID NO: 2), STIF1 (SEQ ID NO: 12), and BMY1 (SEQ ID NO: 22) proteins were used. The switchgrass amino acid sequence of each transcription factor was blasted against the maize proteome (Phytozome-Ensemlb-18). The hits were ranked in order of the alignment score. Next each maize amino acid sequence was aligned to the switchgrass sequence using the alignment feature of the Vector NTI software (Invitrogen) to determine the percent identity between the switchgrass and maize orthologs. A summary of the gene and protein sequences of the maize orthologs is shown in TABLE 1. The CLUSTAL O(1.2.4) multiple sequence alignment tool was used to align each switchgrass transcription factor to its switchgrass homologs and its maize orthologs and these alignments are shown in FIG. 1 (STR1), FIG. 2 (STIF1), and FIG. 3 (BMY1). Key characteristics shared among various maize orthologs of STR1 from switchgrass (SEQ ID NO:2) and the STR1 switchgrass protein itself include a tryptophan at position 24, an arginine at position 38, a region of high identity/similarity between positions 102 and 156, a proline at position 212, a glutamine at position 303, a leucine at position 311, and a proline at position 318, all with numbering of positions relative to STR1 of switchgrass of SEQ ID NO: 2. Key characteristics shared among various maize orthologs of STIF1 from switchgrass (SEQ ID NO: 12) and the STIF1 switchgrass protein itself include a tyrosine at position 4, an alanine at position 25, a histidine at position 37, a region of high identity/similarity between positions 73 and 129, a threonine at position 136, a glycine at position 146, a proline at position 167, a leucine at position 169, a tyrosine at position 172, and an alanine at position 173, all with numbering of positions relative to STIF1 of switchgrass of SEQ ID NO: 12. Key characteristics shared among various maize orthologs of BMY1 from switchgrass (SEQ ID NO: 22) and the BMY1 switchgrass protein itself include a methionine at position 1, a glutamic acid at position 7, a serine at position 8, a glycine at position 9, a region of high identity/similarity between positions 17 and 114, a serine at position 137, a glycine at position 149, and a tyrosine at position 151, all with numbering of positions relative to BMY1 of switchgrass of SEQ ID NO: 22.

TABLE 1
Maize orthologs and homologs to the switchgrass transcription factors STR1, STIF1, and BMY1

| Switchgrass | | | | | |
|---|---|---|---|---|---|
| TF¹ | Maize Ortholog 1 | Maize Ortholog 2 | Maize Ortholog 3 | Maize Ortholog 4 | Maize Ortholog 5 |
| STR1 gene | GRMZM2G018398 | GRMZM2G110333 | GRMZM2G171179 | GRMZM2G018984 | GRMZM2G142179 |
| (Pavir.Ba00410) | SEQ ID NO: 3 | SEQ ID NO: 5 | SEQ ID NO: 7 | SEQ ID NO: 9 | SEQ ID NO: 31 |
| SEQ ID NO: 1 | | | | | |
| STR1 protein | SEQ ID NO: 4 | SEQ ID NO: 6 | SEQ ID NO: 8 | SEQ ID NO: 10 | SEQ ID NO: 32 |
| SEQ ID NO: 2 | (33.2% identity to | (33.1% identity to | (35.1% identity to | (33.7% identity to | (22.4% identity to |
| | switchgrass STR1) | switchgrass STR1) | switchgrass STR1) | switchgrass STR1) | switchgrass STR1) |
| STIF1 gene | GRMZM2G016434 | GRMZM2G087059 | GRMZM2G425798 | GRMZM2G309731 | |
| (Pavir.Aa02595) | SEQ ID NO: 13 | SEQ ID NO: 15 | SEQ ID NO: 17 | SEQ ID NO: 19 | |
| SEQ ID NO: 11 | | | | | |
| STIF1 protein | SEQ ID NO: 14 | SEQ ID NO: 16 | SEQ ID NO: 18 | SEQ ID NO: 20 | |
| SEQ ID NO: 12 | (34.7% identity to | (24.4% identity to | (26.7% identity to | (30.2% identity to | |
| | switchgrass STIF1) | switchgrass STIF1) | switchgrass STIF1) | switchgrass STIF1) | |
| BMY1 gene | GRMZM2G384528 | GRMZM2G180947 | GRMZM2G064426 | GRMZM5G804893 | |
| (Pavir.J05081) | SEQ ID NO: 23 | SEQ ID NO: 25 | SEQ ID NO: 27 | SEQ ID NO: 29 | |
| SEQ ID NO: 21 | | | | | |
| BMY1 protein | SEQ ID NO: 24 | SEQ ID NO: 26 | SEQ ID NO: 28 | SEQ ID NO: 30 | |
| SEQ ID NO: 22 | (78.6% identity to | (76.3% identity to | (45.0% identity to | (45.4% identity to | |
| | switchgrass BMY1) | switchgrass BMY1) | switchgrass BMY1) | switchgrass BMY1) | |

¹gene ID from Phytozome v12.0

Since switchgrass is a tetraploid, available sequence data for switchgrass (Panicum virgatum genotype AP13) available on Phytozome (version 12.1.6) was used to identify additional switchgrass transcription factors with homology to the switchgrass protein sequences of STR1 (SEQ ID NO: 2), STIF1 (SEQ ID NO: 12), and BMY1 (SEQ ID NO: 22). The switchgrass amino acid sequence of each TF was blasted against the switchgrass proteome (Phytozome version 12.1.6). The hits were ranked in order of the alignment score and the top hits are shown in the first column in TABLE 2. These new switchgrass proteins were used to identify new maize orthologs as follows: the switchgrass amino acid sequence of each TF was blasted against the maize proteome (Phytozome-Ensemlb-18) and the hits were ranked in order of the alignment score. Most of the maize orthologs obtained from this process were the same orthologs previously listed in TABLE 1, however three new orthologs including the GRMZM2G457562 protein (SEQ ID NO: 41), the GRMZM2G100727 protein (SEQ ID NO: 43), and the GRMZM2G303465 protein (SEQ ID NO: 48) were identified.

TABLE 2
Switchgrass orthologs to the switchgrass transcription factors
STR1, STIF1, and BMY1 and their maize orthologs and homologs.

| | Maize ortholog 1 | Maize ortholog 2 | Maize ortholog 3 |
|---|---|---|---|
| Switchgrass proteins¹ | | | |
| with homology to STR1 | | | |
| Pavir.Bb03337 protein | GRMZM2G018398 gene | GRMZM2G110333 | GRMZM2G171179 gene |
| (SEQ ID NO: 33) | (SEQ ID NO: 3) | gene (SEQ ID NO: 5) | (SEQ ID NO: 7) |
| | GRMZM2G018398 protein | GRMZM2G110333 | GRMZM2G171179 |
| | (SEQ ID NO: 4) | protein (SEQ ID NO: 6) | protein (SEQ ID NO: 8) |
| Pavir.J04875 protein | GRMZM2G018398 gene | GRMZM2G110333 | GRMZM2G171179 gene |
| (SEQ ID NO: 51) | (SEQ ID NO: 3) | gene (SEQ ID NO: 5) | (SEQ ID NO: 7) |
| | GRMZM2G018398 protein | GRMZM2G110333 | GRMZM2G171179 |
| | (SEQ ID NO: 4) | protein (SEQ ID NO: 6) | protein (SEQ ID NO: 8) |
| Pavir.Aa00281 protein | GRMZM2G018398 gene | GRMZM2G110333 | GRMZM2G171179 gene |
| (SEQ ID NO: 35) | (SEQ ID NO: 3) | gene (SEQ ID NO: 5) | (SEQ ID NO: 7) |
| | GRMZM2G018398 protein | GRMZM2G110333 | GRMZM2G171179 |
| | (SEQ ID NO: 4) | protein (SEQ ID NO: 6) | protein (SEQ ID NO: 8) |
| Pavir.Ib00526 protein | GRMZM2G018984 gene | GRMZM2G018398 | GRMZM2G171179 gene |
| (SEQ ID NO: 36) | (SEQ ID NO: 9) | gene (SEQ ID NO: 3) | (SEQ ID NO: 7) |
| | GRMZM2G018984 protein | GRMZM2G018398 | GRMZM2G171179 |
| | (SEQ ID NO: 10) | protein (SEQ ID NO: 4) | protein (SEQ ID NO: 8) |
| Switchgrass proteins | | | |
| with homology to STIF1 | | | |
| Pavir.Gb01735.1 protein | GRMZM2G425798 gene | GRMZM2G087059 | GRMZM2G016434 gene |
| (SEQ ID NO: 38) | (SEQ ID NO: 17) | gene (SEQ ID NO: 15) | (SEQ ID NO: 13) |
| | GRMZM2G425798 protein | GRMZM2G087059 | GRMZM2G016434 |
| | (SEQ ID NO: 18) | protein (SEQ ID NO: 16) | protein (SEQ ID NO: 14) |
| Pavir.J04335.1 protein | GRMZM2G087059 gene | GRMZM2G457562 | GRMZM2G100727 gene |
| | (SEQ ID NO: 15) | gene (SEQ ID NO: 40) | (SEQ ID NO: 42) |
| (SEQ ID NO: 39) | GRMZM2G087059 protein | GRMZM2G457562 | GRMZM2G100727 |
| | (SEQ ID NO: 16) | protein (SEQ ID NO: 41) | protein (SEQ ID NO: 43) |
| Switchgrass proteins | | | |
| with homology to BMY1 | | | |
| Pavir.Ba00451 protein | GRMZM2G180947 gene | GRMZM2G384528 | GRMZM2G064426 gene |
| (SEQ ID NO: 44) | (SEQ ID NO: 25) | gene (SEQ ID NO: 23) | (SEQ ID NO: 27) |
| | GRMZM2G180947 protein | GRMZM2G384528 | GRMZM2G064426 |
| | (SEQ ID NO: 26) | protein (SEQ ID NO: 24) | protein (SEQ ID NO: 28) |
| Pavir.Ib01924 protein | GRMZM2G180947 gene | GRMZM2G384528 | GRMZM2G064426 gene |
| (SEQ ID NO: 45) | (SEQ ID NO: 25) | gene (SEQ ID NO: 23) | (SEQ ID NO: 27) |
| | GRMZM2G180947 protein | GRMZM2G384528 | GRMZM2G064426 |
| | (SEQ ID NO: 26) | protein (SEQ ID NO: 24) | protein (SEQ ID NO: 28) |
| Pavir.J02009 protein | GRMZM2G064426 gene | GRMZM5G804893 | GRMZM2G303465 gene |
| (SEQ ID NO: 46) | (SEQ ID NO: 27) | gene (SEQ ID NO: 29) | (SEQ ID NO: 47) |
| | GRMZM2G064426 protein | GRMZM5G804893 | GRMZM2G303465 |
| | (SEQ ID NO: 28) | protein (SEQ ID NO: 30) | protein (SEQ ID NO: 48) |
| Pavir.Eb03638 protein | GRMZM2G303465 gene | GRMZM5G804893 | GRMZM2G064426 gene |
| (SEQ ID NO: 49) | (SEQ ID NO: 47) | gene (SEQ ID NO: 29) | (SEQ ID NO: 27) |
| | GRMZM2G303465 protein | GRMZM5G804893 | GRMZM2G064426 |
| | (SEQ ID NO: 48) | protein (SEQ ID NO: 30) | protein (SEQ ID NO: 28) |
| Pavir.J02756 protein | GRMZM2G064426 gene | GRMZM5G804893 | GRMZM2G303465 gene |
| (SEQ ID NO: 50) | (SEQ ID NO: 27) | gene (SEQ ID NO: 29) | (SEQ ID NO: 47) |
| | GRMZM2G064426 protein | GRMZM5G804893 | GRMZM2G303465 |
| | (SEQ ID NO: 28) | protein (SEQ ID NO: 30) | protein (SEQ ID NO: 48) |

¹protein ID from Phytozome v12.1.6

### Example 2. Expression Patterns of Select Transcription Factors in Corn

The in silico expression pattern of select maize orthologs to STR1 (GRMZM2G110333, SEQ ID NO: 5), STIF1 (GRMZM2G016434, SEQ 13) and BMY1 (GRMZM2G384528, SEQ ID NO: 23) were examined using the maize Electronic Fluorescent Pictograph browser (Li, L. et al., Nat Genet, 42 (2010) 1060-1067) (FIG. 4A-C). Surprisingly, the genes for GRMZM2G110333 (SEQ ID NO: 5) and GRMZM2G384528 (SEQ ID NO: 23) were found to have the highest level of expression in developing and whole seed tissue. GRMZM2G016434, (SEQ 13) also had expression in developing seed and whole seed with the highest levels in the 1^{st} leaf and sheath.

The expression of these genes was also experimentally determined by RT-PCR analysis. Maize plants (inbred line B73 obtained from The North Central Regional Plant Introduction Station, Iowa State University) were grown in a greenhouse and tissue at different developmental stages was harvested. The levels of amplification products (FIG. 4D) were measured in 50 ng of total RNA using One Step RT-PCR Kit (Qiagen, Valencia, Calif., USA) as described previously (Somleva, et al., BMC Biotechnol., 14 (2014) 79) using the following pairs of primers: 5′CGTGTTTGGCTTGGTACTTTC3′ and 5′GGAAGTGATGTCTGGTGTCTT3′ for GRMZM2G110333 (SEQ ID NO: 5); TACTCTGACCACGACGATGA and GCAACAACGGAGCTGATACT for GRMZM2G016434 (SEQ ID NO: 13); and 5′GTCGGAGTTCATCTCCTTCATC3′ and 5′ TCATCATGATCATACCGCTTCC3′ for GRMZM2G384528 (SEQ ID NO: 23). Amplification conditions were as follows: 50° C. for 30 min; 95° C. for 15 min; 94° C. for 1 min, 55° C. for 30 sec, 72° C. for 1 min (30 cycles); extension at 72° C. for 15 min. Our experimental examination of the expression pattern of the genes confirmed that GRMZM2G110333 (SEQ ID NO: 5), GRMZM2G016434 (SEQ 13) and GRMZM2G384528 (SEQ ID NO: 23) were expressed in leaves important for providing photoassimilates during seed formation, as well as in the pre-pollination cob and the whole seed 12 days after pollination (FIG. 4D). This suggests a role for the maize transcription factor genes in regulating processes during seed formation that impact seed yield.

### Example 3. Overexpression of Transcription Factors in Corn

Expression cassettes for the maize orthologs of the switchgrass transcription factor proteins STR1 (SEQ ID NO: 2), STIF1 (SEQ ID NO: 12), and BMY1 (SEQ ID NO: 22) can be constructed using a variety of different promoters for expression. Candidate constitutive and seed specific promoters are listed in TABLE 3 and TABLE 4, however those skilled in the art will understand that other promoters can be selected for expression.

TABLE 3
Example promoters for expression in maize

| | | Maize gene ID¹ |
|---|---|---|
| Promoter | Expression | (SEQ ID #)² |
| Hsp70 | Constitutive | GRMZM2G310431 |
| | | (SEQ ID NO: 57) |
| Chlorophyll A/B | Light inducible, | AC207722.2_FG009 |
| Binding Protein | expressed in maize | (SEQ ID NO: 58) |
| (Cab-m5) | mesophyll and | GRMZM2G351977 |
| | bundlesheath cells | (SEQ ID NO: 59) |
| Pyruvate | Constitutive | GRMZM2G306345 |
| phosphate | | (SEQ ID NO: 60) |
| dikinase (PPDK) | | |
| Actin | Constitutive | GRMZM2G047055 |
| | | (SEQ ID NO: 61) |
| ADP-glucose | Seed specific | GRMZM2G429899 |
| pyrophos- | | (SEQ ID NO: 62) |
| phorylase | | |
| (AGPase) | | |
| β- | Seed specific | GRMZM2G139300 |
| fructofuranosidase | | (SEQ ID NO: 63) |
| insoluble | | |
| isoenzyme 1 | | |
| (CIN1) | | |
| Maize MADS box | Seed specific | GRMZM2G160687 |
| promoter | | (SEQ ID NO: 56) |
| Maize trpA | Seed specific | GRMZM5G841619 |
| promoter | | (SEQ ID NO: 74) |

¹Gene ID on Phytozyme v. 12.1.6;
²Promoter region includes the predicted 5′UTR of the gene and 1200 bp of sequence upstream of the 5′UTR in Phytozyme v. 12.1.6

In some instances, it may be advantageous to create a hybrid promoter containing a promoter sequence and an intron. These promoters can deliver higher levels of Mable expression. Examples of such hybrid promoters are listed in TABLE 4.

TABLE 4
Hybrid promoter replacement cassettes

| Promoter | Expression | |
|---|---|---|
| Hybrid maize Cab-m5 | Light inducible, expressed in maize | SEQ ID NO: 64 |
| promoter/maize hsp70 | mesophyll and bundlesheath cells | |
| intron | | |
| Maize ubiquitin | Constitutive (maize ubiquitin promoter | SEQ ID NO: 65 |
| promoter/maize | and intron sequence listed in Genbank | |
| ubiquitin intron | KT962835) | |
| Maize ubiquitin | Constitutive (maize promoter and intron | SEQ ID NO: 70 |
| promoter/maize | sequence with 99% identity to sequence | |
| ubiquitin intron | in Genbank KT985051.1) | |
| Maize ubiquitin | Constitutive | |
| promoter/adh1 intron | | |
| 1 | | |
| Rice actin | Constitutive | |
| promoter/actin intron 1 | | |
| Maize H2B (histone) | Constitutive | |
| promoter/ubiquitin | | |
| intron 1 | | |

Expression cassettes for maize gene GRMZM2G384528 (SEQ ID NO: 23), one of the maize orthologs for the switchgrass BMY1 transcription factor gene (SEQ ID NO: 21) were designed using different promoters to drive expression of the transgenes. YTEN26 (FIG. 5A, SEQ ID NO: 66) is expressed from the hybrid maize cab-m5/maize hsp70 intron promoter (SEQ ID NO: 64) and is flanked by maize hsp70 terminator. The cab-m5 promoter has been previously shown to be light inducible and expressed in both mesophyll and bundlesheath cells of maize, with some preference for mesophyll (Sheen et al., P. Natl. Acad. Sci. USA, 1986, 83, 7811). YTEN27 (FIG. 5B, SEQ ID NO: 67) is expressed from the maize MADS-box promoter (SEQ ID NO: 56) and is flanked at the 3′ end by the hsp70 terminator. YTEN28 (FIG. 5C, SEQ ID NO: 68) is expressed from the maize trpA promoter (SEQ ID NO: 74) and is flanked at the 3′ end by the hsp70 terminator. YTEN29 (FIG. 5D, SEQ ID NO: 69) is expressed from the maize ubiquitin promoter with the maize ubiquitin intron 1 (SEQ ID NO: 65) and is flanked at the 3′ end by the hsp70 terminator.

Maize: Methods for maize transformation are routine and well known in the art and have recently been reviewed by Que et al., (2014), Frontiers in Plant Science 5, article 379, pp 1-19.

Protoplast transformation: Protoplast transformation methods useful for practicing the invention are well known to those skilled in the art. Such procedures include for example the transformation of maize protoplasts as described by Rhodes and Gray (Rhodes, C. A. and D. W. Gray, Transformation and regeneration of maize protoplasts, in Plant Tissue Culture Manual: Supplement 7, K. Lindsey, Editor. 1997, Springer Netherlands: Dordrecht. p. 353-365).

Agrobacterium-mediated transformation: For transformation of maize, fragments from YTEN26, YTEN 27, YTEN28, or YTEN29 can be inserted into a binary vector that also contains an expression cassette for a selectable marker. For example the bar gene imparting the transgenic plants with resistance to bialophos can be used for selection. The binary vector is transformed into an Agrobacterium tumefaciens strain, such as A. tumefaciens strain EHA101.

Agrobacterium-mediated transformation of maize can be performed following a previously described procedure (Frame et al., 2006, Agrobacterium Protocols Wang K., ed., Vol. 1, pp 185-199, Humana Press) as follows.

Plant Material: Plants grown in a greenhouse are used as an explant source. Ears are harvested 9-13 days after pollination and surface sterilized with 80% ethanol.

Explant Isolation, Infection and Co-Cultivation: Immature zygotic embryos (1.2-2.0 mm) are aseptically dissected from individual kernels and incubated in an A. tumefaciens strain EHA101 culture containing the transformation vector of interest for genome editing (grown in 5 ml N6 medium supplemented with 100 μM acetosyringone for stimulation of the bacterial vir genes for 2-5 h prior to transformation) at room temperature for 5 min. The infected embryos are transferred scutellum side up on to a co-cultivation medium (N6 agar-solidified medium containing 300 mg/l cysteine, 5 μM silver nitrate and 100 μM acetosyringone) and incubated at 20° C., in the dark for 3 d. Embryos are transferred to N6 resting medium containing 100 mg/l cefotaxime, 100 mg/l vancomycin and 5 μM silver nitrate and incubated at 28° C., in the dark for 7 d.

Callus Selection: All embryos are transferred on to the first selection medium (the resting medium described above supplemented with 1.5 mg/l bialaphos) and incubated at 28° C. in the dark for 2 weeks followed by subculture on a selection medium containing 3 mg/l bialaphos. Proliferating pieces of callus are propagated and maintained by subculture on the same medium every 2 weeks.

Plant Regeneration and Selection: Bialaphos-resistant embryogenic callus lines are transferred on to regeneration medium I (MS basal medium supplemented with 60 g/l sucrose, 1.5 mg/l bialaphos and 100 mg/l cefotaxime and solidified with 3 g/l Gelrite) and incubated at 25° C. in the dark for 2 to 3 weeks. Mature embryos formed during this period are transferred on to regeneration medium II (the same as regeneration medium I with 3 mg/l bialaphos) for germination in the light (25° C., 80-100 μmol/m²/s light intensity, 16/8-h photoperiod). Regenerated plants are ready for transfer to soil within 10-14 days. Plants are grown in the greenhouse to maturity and T1 seeds are isolated.

The copy number of the transgene insert is determined, through methods such as Southern blotting or digital PCR, and lines are selected to bring forward for further analysis. Overexpression of the transcription factors is determined by RT-PCR and/or Western blotting techniques and plants with the desired level of expression are selected. Homozygous lines are generated. The yield seed of homozygous lines is compared to control lines.

Transformation using nanotubes or nanoparticles: Nanoparticles or nanotubes capable of delivering biomolecules to plants can also be used to practice the invention (for review see Cunningham, 2018, Trends Biotechnol., 36, 882).

Stress experiments, where transgenic plants and their control plants are subjected to drought, nitrogen deficiency, flooding, heat stress, cold stress, and/or salinity, can also be performed to identify transcription factors that provide stress tolerance.

### Example 4. Modulating Expression of Transcription Factors Using CRISPR/Cas Genome Editing Mediated Promoter Replacement

Methods for targeted mutagenesis, precise gene editing, and site-specific gene insertion in maize using Cas9 and guide RNA have recently been published (Svitashev, S., Young, J. K., Schwartz, C., Gao, H., Falco, S. C. and Cigan, A. M. 2015. Plant Physiology 169, 931-945). The expression of a transcription factor can be modulated by replacing the endogenous promoter in front of the transcription factor with a new promoter that is expressed at a higher or lower level, is expressed at a different developmental stage, and/or has a different tissue specificity. To modulate expression of the maize orthologs of the switchgrass transcription factors STR1 (SEQ ID NO: 2), STIF1 (SEQ ID NO: 12), and BMY1 (SEQ ID NO: 22), CRISPR/Cas9 mediated promoter replacement can be used.

Promoter replacement requires the delivery of three elements to the plant, the sgRNAs to target the insertion site, the promoter cassette for insertion that is flanked by regions homologous to the genome insertion site, and the Cas nuclease enzyme. The flanking regions with homology to the genome insertion site enable incorporation of the promoter cassette through the plants endogenous homology directed repair mechanism. Delivery of the necessary genetic elements to enable promoter replacement can be achieved in multiple ways: by introducing a complex of the Cas9 enzyme, the synthesized sgRNAs, and the promoter cassette to be inserted (called ribonucleoprotein complexes, or RNPs) (FIG. 7C) directly to protoplasts (Woo et al., Nature Biotechnology, 2015, 33, 1162-1164); by transfection of protoplasts either stably or transiently with a genetic construct(s) containing expression cassettes for DNA encoding the sgRNA(s) and the Cas9 enzyme, mixed with a DNA fragment containing the promoter to be inserted (FIG. 7B); through particle bombardment of the plant or plant tissues with a genetic construct(s) with expression cassettes for DNA encoding the sgRNA(s) and the Cas9 enzyme, mixed with a DNA fragment containing the promoter to be inserted (FIG. 7B); or through Agrobacterium-mediated transformation of the plant or plant tissues using a binary construct(s) with expression cassettes for DNA encoding the sgRNA(s), the Cas9 enzyme, and the promoter DNA fragment to be inserted (FIG. 7B). For Agrobacterium-mediated transformation, it is advantageous to have the promoter DNA fragment to be inserted flanked by sgRNA binding sites with adjacent PAM sequences, so that Cas9 expression can release the promoter fragment from the vector as it enters the plant, or alternatively can release the promoter fragment from the T-DNA that is stably incorporated into the plant genome.

An advantage of RNPs, as well as the protoplast or particle bombardment methods, with only transient expression of the expression cassettes encoding the Cas9 enzyme and the sgRNAs, is that DNA does not stably integrate into the genome and thus does not need to be removed through segregation to produce a plant containing only the edit. For stable transformation methods, segregation of the unwanted DNA encoding the CRISPR editing machinery must be removed after the edit is obtained by conventional breeding methods. The design of each genetic component to achieve promoter replacement is described below.

Design of single guide RNAs (sgRNAs): The region around the promoter to be replaced in the genome is scanned for protospacer adjacent motif (PAM) sites, sites necessary for Cas9 to bind and cleave the target sequence. These PAM sites flank the 3′ region of the double stranded DNA cut site for the Cas9 enzyme (FIG. 6C).

From the ˜20 nucleotides of DNA sequence upstream from the PAM site, the sequence of the complementary “guide” can be obtained (FIG. 6C). To generate the functional sgRNA sequence, the sequence of the “guide” is combined with the sequence of a guide RNA scaffold (FIG. 6B). Guide RNA scaffolds have been previously described by other researchers (see for example Mali et al. 2013, Science, 339, pp. 823-826; Li et al. 2013, Nature biotechnology, 31, pp. 688-691; Konermann et al., 2015, Nature, 517, p. 583; Jiang et al., 2013, Nucleic acids research, 41, pp. e188-e188) and are well known in the art. The double stranded DNA sequence (FIG. 6A) required to generate the functional sgRNA (FIG. 6B) can be determined from the sequence of the sgRNA and used in a genetic transformation construct.

Ideally, the sequence of the DNA encoding the “guide” (FIG. 6A) is identical to the genomic DNA sequence, or “guide target sequence”, that is base paired to the sgRNA (FIG. 6C). In practice, some mismatches between the sequence of the DNA encoding the guide (FIG. 6A) and the genomic DNA sequence can be tolerated and still result in double stranded cleavage by Cas9.

DNA encoding the guides (FIG. 6A) necessary to generate sgRNAs (FIG. 6B) to excise promoter regions from the maize orthologs of the switchgrass transcription factors STR1 (SEQ ID NO: 1), STIF1 (SEQ ID NO: 11), and BMY1 (SEQ ID NO: 21) were designed by identifying promoter regions upstream of the start codon and the 5′UTR of each ortholog. This typically contained sequence before the ATG of the coding sequence (CDS) that included 1000-1200 bp of sequence upstream of the 5′UTR (TABLE 5, FIG. 7A). Verification that the specific sequence contains a predicted promoter can be performed using the RegSite Plant DB from Softberry Inc. (website: softberry.com/berry.phtml?topic=index&group=programs&subgroup=promoter) or similar programs.

DNA sequences encoding the guide portion of the sgRNA for three sgRNAs are shown in TABLE 5. These DNA sequences are ˜20 nucleotides in length and span different regions of the upstream promoter (FIG. 7A). When fused to DNA encoding the guide RNA scaffold (gRNA Sc) (FIG. 6A), the transcribed product is a functional sgRNA (FIG. 6B) that has all the elements to bind with the complementary target genomic DNA that lies adjacent to a PAM sequence (FIG. 6C) and to interact with the CAS enzyme. The DNA sequences encoding the guide portion of sgRNA shown in TABLE 5 were designed to be components of three sgRNA sequences to target various regions of the endogenous maize promoter of a transcription factor gene. The use of two sgRNAs can allow for targeted excision of a region of the endogenous promoter, which can be the core base elements of the promoter, for example the −10 and −35 regions, or can include a large fragment encompassing the entire promoter region and untranslated regions. The use of a single sgRNA promotes site specific cleavage of DNA within the region of the endogenous promoter. The positions of the upstream promoter region that are targeted by the sgRNA sequences are outlined in FIG. 7A. DNA sequences encoding the guide portion of sgRNA were designed following the SpCas9 guide RNA architectures (equivalent to 20 nucleotides of the target genomic DNA that is adjacent to a PAM sequence of NGG) using a web-based guide RNA design tool, CRISPOR, on the TEFOR website. A number of other web-based tools can also be used for guide sequence selection and analysis, such as CRISPRdirect and CRISPR-P 2.0 (Ding et al., 2016, Frontiers in Plant Science, 7, 703; Naito et al., 2015, Bioinformatics, 31, 1120; Liu et al., 2017, Molecular Plant, 10, 530). Based on the disclosure herein, it will be apparent to a person of skill in the art that different sgRNAs to target different regions of the endogenous promoter for promoter insertion or replacement can also be used to modulate the expression of the maize orthologs of the switchgrass transcription factors STR1 (SEQ ID NO: 1), STIF1 (SEQ ID NO: 11), and BMY1 (SEQ ID NO: 21). Three different DNA sequences encoding guide portions of three different sgRNA are designated as Guide 1, Guide 2, and Guide 3 in TABLE 5. When these sequences are transcribed as part of a DNA molecule containing the sequence encoding the RNA scaffold, they produce a functional sgRNA that targets the regions around the promoter and 5′UTR region for each maize transcription factor listed in TABLE 5. Similar DNA sequences encoding the guide portion of sgRNAs can be designed for all of the maize genes listed in TABLE 1 and TABLE 2 using the upstream promoter sequences described in TABLE 5 and TABLE 6.

TABLE 5
Guide target sequences for Cas9 mediated excision of promoters of transcription factor genes in corn

| | | Length of | | | | | | | | | |
|---|---|---|---|---|---|---|---|---|---|---|---|
| | | upstream | Guide #1² | | | Guide #2 | | | Guide #3 | | |
| | | region from | | Guide | | | Guide | | | Guide | |
| Maize | | CDS used | | sequence | | | sequence | | | sequence | |
| Gene | Gene locus name | for analysis¹ | Strand³ | (5' to 3') | PAM⁴ | Strand | (5' to 3') | PAM | Strand | (5' to 3') | PAM |
| STR1 | GRMZM2G110333 | 1113 | - | GTAAAC | GGG | + | TAGAGTA | TGG | + | CAATTA | AGG |
| ortholog | (SEQ ID NO: 5) | (SEQ ID | | AAATCG | | | GAATTTC | | | CGAGTA | |
| | | NO: 52) | | GTGCTTG | | | AAATGG | | | TTAAAT | |
| | | | | C (SEQ ID | | | (nt 752-771 | | | GC (nt | |
| | | | | NO: 90) | | | of SEQ ID | | | 462-481 | |
| | | | | | | | NO: 52) | | | of SEQ | |
| | | | | | | | | | | ID NO: | |
| | | | | | | | | | | 52) | |
| STR1 | GRMZM2G142179 | 1452 | + | CATACCA | GGG | + | CCGGCTC | AGG | - | AGTAAT | GGG |
| ortholog | (SEQ ID NO: 31) | (SEQ ID | | AAGCGTC | | | AGCTGTC | | | TTCGGG | |
| | | NO: 53) | | GGAAGA | | | ATTTAC | | | ATTCAC | |
| | | | | (nt 1368- | | | (nt 748-767 | | | GA (SEQ | |
| | | | | 1387 of | | | of SEQ ID | | | ID NO: | |
| | | | | SEQ ID | | | NO: 53) | | | 91) | |
| | | | | NO: 53) | | | | | | | |
| STIF1 | GRMZM2G016434 | 1051 | + | TAAAATA | AGG | - | GTGTTTC | AGG | + | GGACCG | AGG |
| ortholog | (SEQ ID NO: 13) | (SEQ ID | | AGATGGT | | | GAACGT | | | AAGGA | |
| | | NO: 54) | | ACAAGA | | | AAACTCG | | | GAGTAA | |
| | | | | (nt 1002- | | | (SEQ ID | | | ATT (nt | |
| | | | | 1021 of | | | NO: 92) | | | 267-286 | |
| | | | | SEQ ID | | | | | | of SEQ | |
| | | | | NO: 54) | | | | | | ID NO: | |
| | | | | | | | | | | 54) | |
| BMY1 | GRMZM2G384528 | 1380 | + | CTCCGCT | CGG | + | GCGTGTT | GGG | - | CAACGG | AGG |
| ortholog | (SEQ ID NO: 23) | (SEQ ID | | CTCTCAA | | | GGCAAG | | | CGACGA | |
| | | NO: 55) | | ACTCCC | | | CCCGCTC | | | AACGA | |
| | | | | (nt 1232- | | | (nt 724-743 | | | GTG | |
| | | | | 1251 of | | | of SEQ ID | | | (SEQ ID | |
| | | | | SEQ ID | | | NO: 55) | | | NO: 93) | |
| | | | | NO: 55) | | | | | | | |

¹Sequence before the ATG of the coding sequence (CDS) of the transcription factor includes at least 1000 bp of sequence upstream of the 5′UTR predicted by Phytozyme and/or transcript analysis, which is a variable length for each gene.
²Guides #1, #2, #3 are DNA molecules encoding the guide portion of sgRNA. They are fused to DNA encoding the guide RNA scaffold (gRNA Sc)(i.e. See FIG 6A) and the resulting transcribed product is a functional sgRNA (FIG. 6B) that has all the elements to bind with the complementary target genomic DNA that lies
adjacent to a PAM sequence (FIG. 6C), and to interact with the CAS enzyme. The sequences of the Guides #1, 2, and 3 are inherently equivalent to the guide target sequence to which the sgRNA base pairs on the genomic DNA for cleavage, and these positions in the upstream region of the endogenous transcription factor promoter are illustrated in FIG. 7A. The term ″nt″ refers to nucleotides at positions within sequences as specified.
³Strand (+/-) refers to the sgRNA binding to either the forward strand of DNA (+) or its reverse complement (-).
⁴PAM refers to the protospacer adjacent motif that resides directly adjacent to the 3′ end of the guide target site (FIG. 6C).

TABLE 6
Promoter regions for additional maize orthologs to switchgrass
transcription factors STR1, STIF, and BMY1

| | | Length of upstream region |
|---|---|---|
| Maize Gene | Gene locus name | from CDS used for analysis¹ |
| STR1 ortholog | GRMZM2G018398 | 1378 |
| | (SEQ ID NO: 3) | (SEQ ID NO: 75) |
| | GRMZM2G171179 | 1387 |
| | (SEQ ID NO: 7) | (SEQ ID NO: 76) |
| | GRMZM2G018984 | 1565 |
| | (SEQ ID NO: 9) | (SEQ ID NO: 77) |
| STIF ortholog | GRMZM2G087059 | 1200 |
| | (SEQ ID NO: 15) | (SEQ ID NO: 78) |
| | GRMZM2G425798 | 1200 |
| | (SEQ ID NO: 17) | (SEQ ID NO: 79) |
| | GRMZM2G309731 | 1200 |
| | (SEQ ID NO: 19) | (SEQ ID NO: 80) |
| | GRMZM2G457562 | 1538 |
| | (SEQ ID NO: 40) | (SEQ ID NO: 81) |
| | GRMZM2G100727 | 1200 |
| | (SEQ ID NO: 42) | (SEQ ID NO: 82) |
| BMY1 ortholog | GRMZM2G180947 | 1689 |
| | (SEQ ID NO: 25) | (SEQ ID NO: 83) |
| | GRMZM2G064426 | 1480 |
| | (SEQ ID NO: 27) | (SEQ ID NO: 84) |
| | GRMZM5G804893 | 1476 |
| | (SEQ ID NO: 29) | (SEQ ID NO: 85) |
| | GRMZM2G303465 | 1200 |
| | (SEQ ID NO: 47) | (SEQ ID NO: 86) |

¹Sequence before the ATG of the coding sequence (CDS) of the transcription factor includes 1200 bp of sequence upstream of the 5′UTR predicted by Phytozyme and/or transcript analysis, which is a variable length for each gene (See FIG. 7).

Design of promoter insertion cassette: The promoter insertion cassette contains the promoter to be inserted flanked by DNA that is homologous to each side of the CRISPR/Cas nuclease cut site. An illustration of a promoter insertion cassette for promoter X is shown in FIG. 7B. The flanking DNA fragments direct the promoter cassette insertion into the cut genomic DNA which is subsequently repaired through the plants endogenous homology directed repair mechanism. In the example in FIG. 7, guide target sites #1 and #3 have been used to excise DNA in the promoter region upstream of the transcription factor. The flanking regions for the promoter insertion cassette are thus designed to be homologous to DNA upstream of the guide target site #3 nuclease cut site and downstream of the guide target site #1 nuclease cut site.

The promoter to be inserted can be selected from the large number of promoters active in plant cells, including the promoters listed in TABLE 3 and TABLE 4. Promoters can be selected based on the desired strength and intended tissue specific expression pattern for the transcription factor. Liu & Stewart (2016, Current Opinion in Biotechnology, 37, 36) have described synthetic promoters that are active in plants cells and these can also be used to enable the invention. Based on the disclosure herein, it will be apparent to a person of skill in the art that TABLE 3 and TABLE 4 represent examples of promoters that can be used and that there are other promoters that are active in plants that can be substituted for these promoters.

Depending on the method for delivering the promoter insertion cassette to the plant, it may be advantageous to flank the insertion cassette with sgRNA binding sequences to release the insertion cassette in the presence of active Cas9 (FIG. 7B). For example, if the insertion cassette is delivered on a plasmid from transfection of protoplasts or particle bombardment transformation procedures, flanking the insertion cassettes with sgRNA binding sites and adjacent PAM sequences will release the insertion cassette in the presence of Cas9. If the delivery method is via Agrobacterium-mediated plant transformation, flanking the insertion cassettes with the sgRNA binding sites and PAM sequences will release the insertion cassette from the T-DNA in the presence of Cas9. This may expedite insertion.

Genetic Constructs for Replacing the Promoter for Expression of GRMZM2G384528 (SEQ ID NO: 23), a Maize Ortholog of the Switchgrass BMY1 Transcription Factor:

Promoter replacement through Agrobacterium-mediated transformation: Binary vector pYTEN30 (FIG. 8A, SEQ ID NO: 71) contains expression cassettes containing the Guide 1 and 3 DNA fragments in TABLE 5. These DNA fragments can each be fused to a DNA fragment encoding a guide RNA scaffold to form functional sgRNAs that together can excise a portion of the endogenous promoter of the switchgrass BMY1 maize ortholog encoded by GRMZM2G384528 (SEQ ID NO: 23) (TABLE 1). The Guide 1 and 3 DNA fragments in TABLE 5 encode the guide portion of a sgRNA and were designed as described in FIG. 7 using a 1380 bp maize genomic DNA fragment downloaded from Phytozome (SEQ ID NO: 55), encompassing the 5′UTR of the GRMZM2G384528 gene plus an additional ˜1 kb DNA upstream of the 5′UTR in the promoter region of GRMZM2G384528. In transformation vector pYTEN30, the Guide 1 and 3 DNA are each fused with DNA encoding sgRNA scaffolds and the resulting DNA fragments are expressed in separate expression cassettes under the control of the rice U6 promoter. Transformation vector pYTEN30 also contains the Cas9 enzyme codon optimized for rice expressed from a double enhanced CaMV 35S promoter, and the hptI gene (containing a CAT-1 intron) for selection of transformants with hygromycin expressed from a double enhanced CaMV 35S promoter fused to an hsp70 intron.

The T0 plants obtained from Agrobacterium transformation are examined for CAS9 mediated DNA insertions as follows: During growth, leaf material from the T0 transformants is harvested and DNA is extracted from the plant tissue using DNA extraction procedures well known in the art. There are multiple commercially available kits, such as the Qiagen Plant DNeasy kit, that can be employed for this purpose. PCR reactions are performed using primers that bind to regions of genomic DNA about 100 base pairs away from the guide #1 and #3 target sites (FIG. 7A). Sequencing analysis is performed on the crude PCR mixture using a Next-Generation sequencing technology and automated sequencing assembly offered by a vendor. Plants with insertions are identified and allowed to grow in a greenhouse to maturity prior to seed harvest (T1 generation).

T1 seeds are planted and grown in a greenhouse, leaf tissue is harvested, and genomic DNA is isolated. Lines are screened for the presence of the selectable marker gene and/or the Cas9 gene by PCR. Plants that no longer have these genes may have lost the DNA encoding the Cas9 machinery but may still retain the DNA insertion. Retention of the edit in plants that have lost the Cas9 gene is performed using Next Generation Sequencing. Screening for loss of the Cas9 gene can also be done by co-expressing a visual marker such as DsRed, a red fluorescent protein from the Discoma genus of coral (Matz et al., 1999, Nat. Biotechnol. 17, 969-973), by placing an expression cassette coding the gene within the T-DNA region of the vector to allow visual detection of seeds that no longer carry the vector encoded transgenes. T1 transgene free plants are thus further screened for edits by extracting genomic DNA from leaf tissue and performing PCR reactions using primers that bind to regions of genomic DNA about 100 base pairs away from the sgRNA binding site. Sequencing analysis is performed on the crude PCR mixture using a Next-Generation sequencing technology and automated sequencing assembly offered by a vendor. Plants with insertions are identified. Lines with identified insertions that do not contain T-DNA containing the Cas9 gene are identified and allowed to grow in a greenhouse to maturity prior to seed harvest (T2 generation). The expression levels of the transcription factor in various tissues is determined. Transcript levels in seedlings, leaves, stem tissues, roots, silks, cobs, and seeds at different developmental stages are determined by RT-PCR using a gene such as β-actin as a reference. There are multiple methods for extracting total RNA, including through the use of commercially available kits, such as the RNeasy Plant Mini Kit from Qiagen (Valencia, Calif., USA). The RNAeasy Plant Mini Kit from Qiagen is used according to the manufacturer's protocol. DNase treatment and column purification are performed and RNA quality is assessed using an Agilent 2100 Bioanalyzer (Agilent Technologies, Santa Clara, Calif., USA) according to the manufacturer's instructions. The RT-PCR analysis is performed with 50 ng of total RNA using a One Step RT-PCR Kit (Qiagen, Valencia, Calif., USA). Lines with increased expression of the transcription factor are selected and evaluated for yield and stress tolerance as described above.

If required, lines can be grown another generation to obtain homozygous plants with the DNA insertion.

Maize lines are evaluated for their total seed yield and other agronomic parameters such as drought tolerance, stress tolerance, stem thickness, number of cobs, size of cobs. The 100 seed weight of the maize seed can also be analyzed and high yielding lines or lines with good agronomic parameters indicating improved performance as compared the control plants are advanced.

Promoter replacement through protoplast transfections: Construct YTEN31 (FIG. 8B, SEQ ID NO: 72) is a non-binary vector designed for removal of the endogenous promoter from GRMZM2G384528 (SEQ ID NO: 23) and replacement of the endogenous promoter with the maize ubiquitin promoter and intron (SEQ ID NO: 70). This construct can be transformed into maize. The production of maize protoplasts and their transformation has been previously described by Rhodes and Gray (Rhodes, C. A. and D. W. Gray, Transformation and regeneration of maize protoplasts, in Plant Tissue Culture Manual: Supplement 7, K. Lindsey, Editor. 1997, Springer Netherlands: Dordrecht. p. 353-365).

Promoter replacement through genetic transformation through biolistic procedures: Site directed insertion of a DNA fragment into maize embryos through homology directed repair using biolistic transformation procedures has been previously described by Svitashev et al. (2015, Plant Physiol., 169, 931). Construct YTEN31 can be used for promoter replacement of the GRMZM2G384528 (SEQ ID NO: 23) using the procedures for generation of maize embryos, biolistic transformation, and regeneration of plants as described by Svitashev et al. Alternatively, nanotube or nanoparticle mediated DNA delivery can be used (Kwak et al., 2019, Nature Nanotechnology, DOI 10.1038/s41565-019-0375-4) (Demirer et al, 2019, Nature Nanotechnology, DOI 10.1038/s41565-019-0382-5),

Promoter replacement using ribonucleoprotein complexes: Ribonucleoprotein complexes (RNPs) of Cas9, synthesized sgRNAs, and promoter insertion cassettes, can be delivered to the appropriate plant tissue to achieve promoter replacement. In some cases, appropriate tissue will be protoplasts due to the ease of uptake of the RNPs, and the ability to produce callus cultures from the protoplasts which can subsequently be regenerated into plants using appropriate tissue culture methods. Woo et al. (Nature Biotechnology, 2015, 33, 1162-1164) have described the delivery of RNPs to plant protoplasts and subsequent genome editing. RNPs can also be delivered using methods employing for example nanotubes.

DNA construct YTEN32 (FIG. 8C, SEQ ID NO: 73) is designed as a promoter insertion cassette to replace the endogenous promoter of GRMZM2G384528 (SEQ ID NO: 23), a maize ortholog of the switchgrass BMY1 transcription factor, with the maize ubiquitin promoter (SEQ ID NO: 70). Formation of RNPs using the DNA fragment of YTEN32, purified CAS9 enzyme, and two synthesized sgRNAs to remove a portion of the GRMZM2G384528 promoter can be used. The two synthesized sgRNAs are produced containing a guide and scaffold. One sgRNA contains the transcribed Guide #3 sequence for GRMZM2G384528 (TABLE 5) fused to a guide RNA scaffold to form a functional chimeric guide RNA. The other sgRNA contains the transcribed Guide #1 sequence for GRMZM2G384528 (TABLE 5) fused to a guide RNA scaffold to form a functional chimeric single guide RNA (sgRNA). RNPs are formed as described by Woo et al. (Nature Biotechnology, 2015, 33, 1162-1164) and delivered to maize protoplasts that are made as previously described by Rhodes and Gray (Rhodes, C. A. and D. W. Gray, Transformation and regeneration of maize protoplasts, in Plant Tissue Culture Manual: Supplement 7, K. Lindsey, Editor. 1997, Springer Netherlands: Dordrecht. p. 353-365).

Cell-penetrating peptides can also be used to deliver RNPs into cells. The delivery of macromolecules with cell-penetrating peptides has previously been demonstrated in triticale (Chugh et al., 2009, Plant Cell Rep., DOI 10.1007/s00299-009-0692-4) and permeabilized wheat immature embryos (Chugh and Eudes, 2008, FEBS J., 10, 2403) and can be adapted for use in maize.

### Example 5

The expression of a transcription factor can be modulated by insertion of various genetic elements. The replacement of the promoter in front of the transcription factor is described above. Other methods for modulating promoter activity include insertion of an intron near the 5′ end of the transcription factor gene to achieve more stable expression, or insertion of a transcriptional enhancer sequence to modify the activity of the promoter. Examples of such insertion cassettes and their insertion in plant genomic DNA to modify the strength of a promoter are illustrated in FIG. 9.

There are multiple intron sequences that can be used to enable the invention, including the HSP70 intron (Brown & Santino, 1997, U.S. patent Ser. No. 05/593,874) and the maize ubiquitin 1 intron.

There are multiple enhancer sequences that can be used to enable the invention that are capable of enhancing the activity of a plant promoter, including the enhancer element of the 35S promoter (Kay et al., 1987, Science, 1987, 236, 1299).

### Example 6. CRISPR Editing with the CpfI Nuclease

In some cases, it may be desirable to use a nuclease with a different PAM sequence than the Cas9 enzyme to enable insertion of DNA into plant genomes. The CpfI class of enzymes have a different PAM sequence, depending on their source, allowing cuts at different genomic sequences than Cas9, which has a PAM sequence of “NGG”. There are several CpfI enzymes available (Zetsche et al., 2015, Cell, 163, 759; Gao et al., 2017, Nature Biotech., doi:10.1038/nbt.3900; Tang et al., 2017, Nat Plants, 3, Article number 17018; Wang et al., Molecular Plant, 2017, 10, 1011; Begemann et al., 2017, Scientific Reports, 7, 11606), some which are listed in TABLE 7 with their corresponding PAM sequences, all of which are useful for practicing this invention.

The CpfI enzyme produces double stranded DNA breaks with nucleotide overhangs, whereas Cas9 produces blunt ends. Engineering similar nucleotide overhangs on the DNA fragment to be inserted might improve insertion (Li et al., 2018, Journal of Experimental Botany, 69, 4715). CpfI enzyme also does not need a tracrRNA to be functional, thus sgRNAs for this enzyme are shorter.

Examples of using Cpf1 enzymes for genome insertion in plants include Begemann et al. (2017, Scientific Reports, 7, 11606), Li et al. (2018, Journal of Experimental Botany, 69, 4715).

TABLE 7
Cpf1 enzymes and their variants useful for genome editing

| Cpf1 Enzyme | Source | PAM¹ |
|---|---|---|
| AsCpf1 | Acidaminococcus sp. | TTTV |
| | BV3L6 | |
| AsCpf1 S542R/K607R | AsCpf1 variant | TYCV |
| AsCpf1 S542R/K548V/N552R | AsCpf1 variant | TATV |
| LbCpf1 | Lachnospiraceae | TTTV |
| | bacterium ND2006 | |
| LbCpf1 G532R/K595R | LbCpf1 variant | TYCV |
| FnCpf1 | Francisella novicida | TTN |
| | U112(NC_008601) | |

¹Abbreviations in PAM consensus sequences; Y = C or T; V = A, C, or G; N = any base

The ability of the CpfI enzyme to cleave its own CRISPR RNA also allows an array of sgRNAs to be arranged on a single genetic fragment which is subsequently cleaved by CpfI to initiate multiplex editing (Zetsche et al., 2017, Nature Biotech, 35, 31-34).

### REFERENCE TO A “SEQUENCE LISTING,” A TABLE, OR A COMPUTER PROGRAM LISTING APPENDIX SUBMITTED AS AN ASCII TEXT FILE

The material in the ASCII text file, named “YTEN-58988WO-Sequence-Listing_ST25.txt”, created Apr. 1, 2019, file size of 233,472 bytes, is hereby incorporated by reference.

1. A method for modifying a corn plant, the method comprising upregulating, in the corn plant, one or more polynucleotides or polypeptides selected from among the following:
(a) one or more polypeptides comprising SEQ ID NOS: 87, 88, or 89;
(b) one or more polypeptides comprising SEQ ID NOS: 4, 6, 8, 10, 14, 16, 18, 20, 24, 26, 28, 30, 32, 41, 43 or 48;
(c) one or more of the polypeptides set forth in (a) having at least 85%, 90%, 95% or higher sequence identity to one or more of the polypeptides set forth in (b);
(d) one or more polynucleotides comprising SEQ ID NOS: 3, 5, 7, 9, 13, 15, 17, 19, 23, 25, 27, 29, 31, 40, 42 or 47;
(e) one or more polynucleotides having at least 85%, 90%, 95% or higher sequence identity to one or more of the polynucleotides set forth in (d); or
(f) one or more polypeptides encoded by one or more of the polynucleotides set forth in (d) or (e).

2. The method of claim 1, further comprising growing the modified plant under conditions whereby the modified plant exhibits one or more enhanced characteristics as compared to a control plant grown under similar conditions.

3. The method of claim 1, wherein the one or more upregulated polynucleotides comprise SEQ ID NOS: 3, 5, 7, 9, 13, 15, 17, 19, 23, 25, 27, 29, 31, 40, 42 or 47.

4. The method of claim 1, wherein the one or more upregulated polynucleotides or polypeptides exhibit at least a two-fold change in expression as compared to that of a control plant.

5. The method of claim 4, wherein the change in expression is accomplished by introducing a transgene for one or more global transcription factors, wherein the transgene comprises a polynucleotide selected from SEQ ID NOS: 3, 5, 7, 9, 13, 15, 17, 19, 23, 25, 27, 29, 31, 40, 42 or 47.

6. The method of claim 1, wherein the one or more upregulated polynucleotides or polypeptides are upregulated by insertion and/or substitution of one or more nucleotides, site-specific mutagenesis, chemical mutagenesis, targeting induced local lesions in genomes (TILLING), gene editing techniques using CRISPR nuclease selected from Cas nuclease, Cas9 nuclease, CasX nuclease, CasY nuclease, a Cpf1 nuclease, a C2c1 nuclease, a C2c2 nuclease (Cas13a nuclease), or a C2c3 nuclease, NgAgo nuclease, TALEN or ZFN techniques.

7. The method of claim 6, wherein the one or more upregulated polynucleotides or polypeptides are upregulated by targeting one or more guide polynucleotides to one or more target sites selected from a promoter, a terminator, or a coding sequence of the one or more polynucleotides set forth in (d) or (e).

8. The method of claim 1, wherein the modified plant exhibits one or more enhanced characteristics selected from higher photosynthesis rates, higher photosynthetic electron transport rates, higher non-photochemical quenching, reduced photorespiration rates, higher biomass yield or content, higher seed yield, improved harvest index, higher seed oil content, improved nutritional composition, improved nitrogen use efficiency, drought resistance, flood resistance, disease resistance, salt tolerance, higher CO₂ assimilation rate, or lower transpiration rate.

9. The method of claim 8, wherein the modified plant exhibits an increase in seed oil content or seed yield as compared to a control plant.

10. The method of claim 9, wherein the seed oil content of the modified plant is increased by 10%, 20%, 30%, 40%, 50%, 60%, 70%, 80%, 90%, 100% or higher relative to the control plant.

11. The method of claim 8, wherein the modified plant exhibits an increase in photosynthetic electron transport rate as compared to a control plant.

12. The method of claim 11, wherein the photosynthetic electron transport rate of the modified plant is increased by 5%, 10%, 20%, 30%, 40%, 50%, 60%, 70%, 80%, 90%, 100% or higher relative to the control plant.

13. A modified corn plant comprising:
(a) one or more polypeptides comprising SEQ ID NOS: 87, 88, or 89;
(b) one or more polypeptides comprising SEQ ID NOS: 4, 6, 8, 10, 14, 16, 18, 20, 24, 26, 28, 30, 32, 41, 43 or 48;
(c) one or more of the polypeptides set forth in (a) having at least 85%, 90%, 95% or higher sequence identity to one or more of the polypeptides set forth in (b);
(d) one or more polynucleotides comprising SEQ ID NOS: 3, 5, 7, 9, 13, 15, 17, 19, 23, 25, 27, 29, 31, 40, 42 or 47;
(e) one or more polynucleotides having at least 85%, 90%, 95% or higher sequence identity to one or more of the polynucleotides set forth in (d); or
(f) one or more polypeptides encoded by one or more of the polynucleotides set forth in (d);
wherein the one or more polypeptides of (a), (b), (c), or (f) or the one or more polynucleotides of (d) or (e) are upregulated.

14. The modified plant of claim 13, wherein the modified plant exhibits one or more enhanced characteristics as compared to a control plant grown under similar conditions.

15. The modified plant of claim 13, wherein the one or more upregulated polynucleotides or polypeptides exhibit at least a two-fold change in expression as compared to that of a control plant.

16. The modified plant of claim 15, wherein the change in expression is accomplished by introducing a transgene for one or more global transcription factors, wherein the transgene comprises a polynucleotide selected from SEQ ID NOS: 3, 5, 7, 9, 13, 15, 17, 19, 23, 25, 27, 29, 31, 40, 42 or 47.

17. The modified plant of claim 13, wherein the one or more upregulated polynucleotides or polypeptides are upregulated by insertion and/or substitution of one or more nucleotides, site-specific mutagenesis, chemical mutagenesis, targeting induced local lesions in genomes (TILLING), gene editing techniques using CRISPR nuclease selected from Cas nuclease, Cas9 nuclease, CasX nuclease, CasY nuclease, a Cpf1 nuclease, a C2c1 nuclease, a C2c2 nuclease (Cas13a nuclease), or a C2c3 nuclease, NgAgo nuclease, TALEN or ZFN techniques

18. The modified plant of claim 17, wherein the one or more upregulated polynucleotides or polypeptides are upregulated by targeting one or more guide polynucleotides to one or more target sites selected from a promoter, a terminator, or a coding sequence of the one or more polynucleotides set forth in (d) or (e).

19. The modified plant of claim 13, wherein the modified plant comprises one or more enhanced characteristics selected from higher photosynthesis rates, higher photosynthetic electron transport rate, higher non-photochemical quenching, reduced photorespiration rates, higher biomass yield or content, higher seed yield, improved harvest index, higher seed oil content, improved nutritional composition, improved nitrogen use efficiency, drought resistance, flood resistance, disease resistance, salt tolerance, higher CO₂ assimilation rate, or lower transpiration rate.

20. The modified plant of claim 19, wherein the modified plant exhibits an increase in seed oil content or seed yield as compared to a control plant.

21-25. (canceled)
